(19) United States
(12) Patent Application Publication
(10) Pub. No.: US 2005/0222408 A1
(43) Pub. Date: Oct. 6, 2005

(54) Heterocyclic amides with anti-tuberculosis activity

(75) Inventors: Richard E. Lee, Memphis, TN (US); Rajendra Prasad Tangallapally, Hanamkonda (IN); Raghunandan Yendapally, Memphis, TN (US); Michael McNeil, Fort Collins, CO (US); Anne Lenaerts, Fort Collins, CO (US)

Correspondence Address: JENKINS, WILSON & TAYLOR, P. A., 3100 TOWER BLVD, SUITE 1400, DURHAM, NC, 27707, US

(73) Assignee: University of Tennessee Research Foundation

(21) Appl. No.: 11/044,420

(22) Filed: Jan. 27, 2005

Related U.S. Application Data
(63) Continuation-in-part of application No. 10/890,750, filed on Jul. 14, 2004.
(60) Provisional application No. 60/487,004, filed on Jul. 14, 2003.
(60) Provisional application No. 60/551,409, filed on Mar. 9, 2004.

(51) Int. Cl. C07D 417/02; C07D 413/02

(52) U.S. Cl. 544/60; 544/152; 544/374; 546/207

(57) ABSTRACT

Compounds having the general structure: 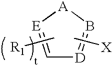
wherein A is selected from the group consisting of oxygen, sulfur, and NR₁₅, and R₁₅ is selected from the group consisting of H, alkyl, aryl, substituted alkyl, and substituted aryl; B,D, and E are each independently selected from the group consisting of CH, nitrogen, sulfur and oxygen; R₁ is selected from the group consisting of nitro, halo, alkyl ester, arylsulfanyl, arylsulfinyl, arylsulfonyl and sulfonic acid; t is an integer from 1 to 3; and X is a substituted amide, and methods of using the novel compounds for treating infections caused by microorganisms, including Mycobacterium tuberculosis.

### RELATED APPLICATIONS

This application is a continuation-in-part and claims the benefit of copending U.S. patent application Ser. No. 10/890,750, which claims priority to U.S. Provisional Patent Application Ser. No. 60/487,004, filed Jul. 14,2003 and U.S. Provisional Patent Application Ser. No. 60/551,409, filed Mar. 9, 2004; the disclosures of all of which are incorporated herein by reference in their entireties.

### GOVERNMENT INTEREST

This invention was made in part with support from grant numbers AI-053796 and NO1-AI-95385 from the National Institutes of Health. The United States government has certain rights in the presently disclosed subject matter.

### TECHNICAL FIELD

The presently disclosed subject matter relates to methods of combating microbial infections with novel compounds. More particularly, the presently disclosed subject matter relates to novel amide compounds and methods of combating microbial infections caused by Mycobacterium tuberculosis using the novel compounds.

### ABBREVIATIONS

Ag.=aqueous
Boc=t-butyloxycarbonyl
CAT=catalyst
CDCl₃=deuterated chloroform
CPBA=chloroperoxybenzoic acid
DEAD=diethyl azodicarboxylate
dil.=dilute
DMAP=4-dimethylaminopyridine
DMF=dimethyl formamide
DMP=Dess-Martin periodinane
DMSO=dimethylsulfoxide
El=electron-impact ionization
EMS=Ethambutyl
ESI=electrospray ionization
Et=ethyl
EtOAc=ethyl acetate
Glf=UDP-galactose mutase
HIV=human immunodeficiency virus
HPLC=high-performance liquid chromatography
Hz=hertz
IC₅₀=Inhibitory concentration
INH=isoniazid
J=spin coupling constant
kg=kilogram
KHMDS=potassium hexamethyidisilazide
M. tuberculosis=Mycobacterium tuberuculosis
MDRTB=multidrug resistant tuberculosis
μg=microgram
mg=milligram
MH_{z}=megahertz
MIC=minimum inhibitory concentration
mL=milliliter
mM or mmol=millimolar
MTD=maximum tolerated dose
NE=no drug
NET=nitrofurantoin
nm=nanometer
NMR=nuclear magnetic resonance
OAc=acetate
OD=optical density
pet. ether=petroleum ether
Py=pyridine
Red-Al=sodium bis(2-methoxyethoxy)aluminum hydride
RMP=rifampin
SM=streptomycin sulfate
solu.=solution
TB=tuberculosis
TFA=trifluoroacetic acid
THF=tetrahydrofuran
TLC=thin layer chromatography
UV=ultraviolet

### BACKGROUND ART

The global burden of tuberculosis (TB) is immense. In 1997 there were an estimated 7.96 million new and 16.2 million existing cases with the worldwide mortality rate at 23%. HIV infection is a key risk factor in TB reactivation rates. HIV infected patients have an elevated risk of primary or reactivated TB, and such active infectious process may enhance HIV replication and increase risk of death. An additional major concern is the rise of multidrug resistant tuberculosis (MDRTB). No new effective treatments have been developed since the introduction of Rifampin in 1971, even though there have been significant advances in drug development technologies. Consequently there is an urgent need to develop new, potent, fast-acting anti-tuberculosis drugs with low toxicity profiles. There is also a need to develop anti-tuberculosis drugs that can be used in conjunction with drugs used to treat HIV infections.

The mycobacterial cell wall is a complex and intriguing mixture of unique components, which sets mycobacteria apart from all other known bacteria. Many of the tuberculosis bacilli characteristics, such as its relatively small size, the ability to grow in macrophages, drug resistance and hydrophilicity are believed to result from components within ultrastructure of the cell wall. Since many of the structural components of the cell wall are not found in humans, enzymes involved in cell wall biosynthesis have proved to be a very fertile ground for the development of anti-tuberculosis drugs. Current anti-tuberculosis drugs such as isoniazid, ethionamide and ethambutol are all believed to act against mycobacterial cell wall biosynthesis, validating the enzymes of cell wall biosynthesis as targets for further drug development.

Thus, there continues to be a need for improvement in the art for additional compounds having desirable anti-microbial activity, whether against the representative pathogens referenced above or against other pathogens. Of particular interest would be compounds having activity in the treatment of human tuberculosis, an infectious disease for which new treatments for multidrug resistant organisms associated with the disease are a particular need in the art.

### SUMMARY

The presently disclosed subject matter provides compounds having the following general structure: 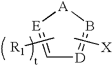
wherein A is selected from the group consisting of oxygen, sulfur, and NR₁₅, wherein R₁₅ is selected from the group consisting of H, alkyl, aryl, substituted alkyl, and substituted aryl; B,D, and E are each independently selected from the group consisting of CH, nitrogen, sulfur and oxygen; R₁ is selected from the group consisting of nitro, halo, alkyl ester, arylsulfanyl, arylsulfinyl, arylsulfonyl and sulfonic acid; t is an integer from 1 to 3; and X is a substituted amide.

In some embodiments, “X” has the following general structure: 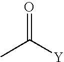
wherein Y is a substituted amine. In some embodiments, Y is an aromatic monoamine.

In some embodiments, the presently disclosed subject matter provides novel compounds having a formula as follows: 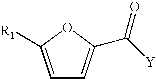
wherein R₁ is selected from the group consisting of halo, nitro, alkyl ester, arylsulfanyl, arylsulfinyl, arylsulfonyl and sulfonic acid; and Y is a substituted amine.

In some embodiments, Y has the general formula —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached.
(a) In some embodiments, Y has the general formula: 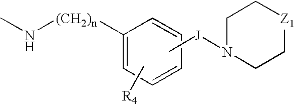
   wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 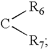
   wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl. In some embodiments n is 1. In some embodiments, R₄ is hydrogen. In some embodiments J is —(CH₂)_{q}—CH═N— and q is 0. In some embodiments, Z₁ is NR₅ and R₅ is an alkyl.

In some embodiments Y has the general formula: 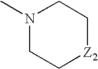
wherein Z₂ is selected from the group consisting of oxygen, NR₁₀, and 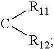
R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl. In some embodiments, Z₂ is NR₁₀ and R₁₀ is a substituted aryl. Further, in some embodiments R₁₀ is: 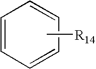
and R₁₄ is selected from the group consisting of halo, alkyl, aryl, substituted alkyl, and substituted aryl.

In some embodiments, Y has the general formula: 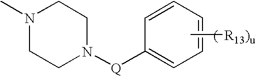
wherein Q is selected from the group consisting of (CH₂)ₛ and —C(═O)— and s is an integer from 0 to 8; u is an integer from 1 to 5; and R₁₃ is selected from the group consisting of hydrogen, halo, alkyl, substituted alkyl, aryl, and substituted aryl. In some embodiments Q is —C(═O)—. In some embodiments, R₁₃ is a halo. In other embodiments R₁₃ is an alkoxyl.

In some embodiments, Y comprises the formula: 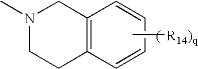
wherein q is an integer from 1 to 4; and R₁₄ is selected from the group consisting of hydrogen, alkyl, substituted alkyl, aryl, and substituted aryl.

In some embodiments, Y comprises:

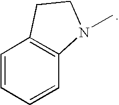

In some embodiments, Y comprises the formula: 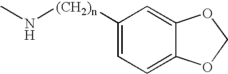
wherein n is an integer from 0 to 8.

In some embodiments, Y comprises the formula: 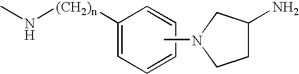
wherein n is an integer from 0 to 8.

The presently disclosed subject matter further describes methods of using the novel compounds disclosed herein. In some embodiments, the presently disclosed subject matter comprises methods of killing or inhibiting the growth of a microorganism comprising contacting the microorganism with an effective amount of one or more of the novel compounds. In some embodiments, the microorganism is a member of the genus Mycobacterium. More particularly, in some embodiments, the microorganism is Mycobacterium tuberculosis.

In some embodiments, the presently disclosed subject matter comprises methods of treating a microbial infection in a subject comprising administering a therapeutically effective amount of one or more of the novel compounds disclosed herein. In some embodiments, the microbial infection is caused by a member of the genus Mycobacterium. More particularly, in some embodiments, the member is Mycobacterium tuberculosis.

The presently disclosed subject matter further encompasses pharmaceutical formulations comprising one or more novel compounds disclosed herein in a pharmaceutically acceptable carrier. In some embodiments, the pharmaceutical formulation is acceptable for intravenous administration and/or oral administration.

It is accordingly an object of the presently disclosed subject matter to provide compounds that are useful in the treatment of microbial infections. It is another object of the invention to provide pharmaceutical formulations for use in the treatment of microbial infections. It is still another object of the invention to provide methods for treating microbial infections.

Some of the objects of the presently disclosed subject matter having been stated hereinabove, and which are addressed in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 shows MIC data for two nitrofuranyl amides, 4 classical TB drugs, and nitrofurantoin. Abbreviations; ethambutol (EMS), isoniazid (INH), nitrofurantoin (NET), rifampin (RMP), streptomycin sulfate (SM), no drug (NE)). Drugs were serially diluted two-fold across 24 wells. The concentrations are reported in μg/ml and are shown above each column.

FIG. 2 shows a summary of the preliminary nitrofuranyl amide series development.

FIG. 3 shows alternative heterocyclic novel compounds disclosed herein.

### DETAILED DESCRIPTION

The presently disclosed subject matter will now be described more fully hereinafter with reference to the accompanying Examples, in which preferred embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

I. Definitions

The term “independently selected” is used herein to indicate that the R groups, e.g., R₂, and R₃ can be identical or different (e.g., R₂ and R₃ may both be substituted alkyls, or R₂ may be hydrogen and R₃ may be a substituted aryl, etc.).

A named “A”, “B”, “D”, “E”, “J”,“R”, “X”, “Y” or “Z” group (and indeed any other referred to by a letter variable) will generally have the structure that is recognized in the art as corresponding to a group having that name, unless specified otherwise herein. For the purposes of illustration, certain representative “A”, “B”, “D”, “E”, “J”, “R,” “X”, “Y” and “Z” groups as set forth above are defined below. These definitions are intended to supplement and illustrate, not preclude, the definitions known to those of skill in the art.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups. Specific examples of acyl groups include acetyl and benzoyl.

“Acylamino” refers to an acyl-NH— group wherein acyl is as previously described.

“Acyloxyl” as used herein refers to an acyl-O— group wherein acyl is as previously described.

As used herein, the term “alkyl” means C₁-C₂₀ inclusive (e.g., 1, 2, 3, 4, 5, 6, 7, 8, 9, 10, 11, 12, 13, 14, 15, 16, 17. 18, 19. or 20 carbon atoms), linear, branched, or cyclic, saturated or unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups.

The alkyl group can be optionally substituted (a “substituted alkyl”) with one or more alkyl group substituents which can be the same or different, where “alkyl group substituent” includes alkyl, halo, arylamino, acyl, hydroxy, aryloxy, alkoxyl, alkylthio, aryl, arylthio, aralkyloxy, aralkylthio, carboxy, alkoxycarbonyl, oxo, ester and cycloalkyl. Representative substituted alkyls include, for example, benzyl, trifluoromethyl and the like. There can be optionally inserted along the alkyl chain one or more oxygen, sulfur or substituted or unsubstituted nitrogen atoms, wherein the nitrogen substituent is hydrogen, alkyl (also referred to herein as “alkylaminoalkyl”), or aryl. “Branched” refers to an alkyl group in which an alkyl group, such as methyl, ethyl or propyl, is attached to a linear alkyl chain.

“Alkoxyl” or “alkoxyalkyl” as used herein refer to an alkyl-O— group wherein alkyl is as previously described. The term “alkoxyl” as used herein can refer to C₁₋₂₀ inclusive (e.g., 1, 2, 3, 4, 5, 6, 7, 8, 9, 10, 11, 12, 13, 14, 15, 16, 17, 18, 19, or 20 carbon atoms), linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Alkoxycarbonyl” as used herein refers to an alkyl-O—CO— group. Exemplary alkoxycarbonyl groups include methoxycarbonyl, ethoxycarbonyl, butyloxycarbonyl, and t-butyloxycarbonyl (Boc).

“Alkylcarbamoyl” as used herein refers to a R′RN—CO— group wherein one of R and R′ is hydrogen and the other of R and R′ is alkyl as described herein.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group can be also optionally be unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

As used herein, the term “amide” refers to a group having the amide functional group:

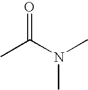

The amide group can be optionally substituted (a “substituted amide”) with one or more amide group substituents which can be the same or different, where “amide group substituent” includes but is not limited to alkyl, halo, arylamino, acyl, hydroxy, aryloxy, alkoxyl, alkylthio, aryl, arylthio, aralkyloxy, aralkylthio, carboxy, alkoxycarbonyl, oxo, ester and cycloalkyl. In some embodiments, the amide group is substituted as described in detail below and throughout the specification, including the Examples and claims.

The terms “amine” and “amino” are used herein to refer to the group —NZ₁Z₂, where each of Z₁ and Z₂ is independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, such as phenylamine (aniline), and methoxyaniline (anisidine), alkoxyl, aryloxyl, silyl, furfuryl, and combinations thereof. Additionally, the amine or amino group may be represented as N+Z₁ Z₂ Z₃, i.e., a quaternary nitrogen group, with the previous definitions applying, and Z₃ being one of H and alkyl. Thus, substituted and unsubstituted amines of the compounds described herein may be primary amines, secondary amines, or tertiary amines, or quaternary ammonium salts.

As used herein, the term “aniline” refers to phenylamine.

As used herein, the term “anisidine” refers to a phenyl group substituted with an amine at one carbon atom and a methyl ether at another carbon atom, e.g. 2-methoxyaniline, 4-methoxyaniline, etc.

As used herein, the term “aralkyl” refers to an aryl-alkyl-group wherein aryl and alkyl are as previously described. Exemplary aralkyl groups include benzyl, phenylethyl, and naphthylmethyl.

“Aroylamino” refers to an aroyl-NH-group, wherein aroyl is defined as an acyl radical derived from an aromatic carboxylic acid, i.e. an arylcarbonyl substituent group, such as benzoyl.

The term “aryl” is used herein to refer to an aromatic substituent which may be a single aromatic ring, or multiple aromatic rings that are fused together, linked covalently, or linked to a common group, such as a methylene or ethylene moiety. The common linking group may also be a carbonyl, as in benzophenone, or oxygen, as in diphenylether, or nitrogen, as in diphenylamine. The term “aryl” specifically encompasses heterocyclic aromatic compounds. The aromatic ring(s) may comprise phenyl, naphthyl, biphenyl, diphenylether, diphenylamine and benzophenone, among others. In some embodiments, the term “aryl” means a cyclic aromatic comprising about 5 to about 10 carbon atoms, e.g. 5, 6, 7, 8, 9, or 10 carbon atoms, and including 5 and 6-membered hydrocarbon and heterocyclic aromatic rings.

The aryl group can be optionally substituted (a “substituted aryl”) with one or more aryl group substituents which can be the same or different, where “aryl group substituent” includes alkyl, aryl, aralkyl, hydroxyl, alkoxyl, aryloxyl, aralkoxyl, carboxy, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene and —NR′R″, where R′ and R″ can be each independently hydrogen, alkyl, aryl and aralkyl.

Specific examples of aryl groups include but are not limited to cyclopentadienyl, phenyl, furan, thiophene, pyrrole, pyran, pyridine, imidazole, isothiazole, isoxazole, pyrazole, pyrazine, triazine, pyrimidine, quinoline, isoquinoline, indole, and the like.

“Aralkoxycarbonyl” as used herein refers to an aralkyl-O—CO— group. An exemplary aralkoxycarbonyl group is benzyloxycarbonyl.

“Aryloxycarbonyl” as used herein refers to an aryl-O—CO-group. Exemplary aryloxycarbonyl groups include phenoxy- and naphthoxy-carbonyl.

As used herein, the term “Aryloxyl” refers to an aryl-O— group wherein the aryl group is as previously described. The term “aryloxyl” as used herein can refer to phenyloxyl or hexyloxyl, and alkyl, halo, or alkoxyl substituted phenyloxyl or hexyloxyl.

“Carbamoyl” as used herein refers to an H₂N—CO-group.

As used herein, the terms “Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group can be also optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, lower alkyl, or aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

A structure represented generally by the formula 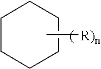
as used herein refers to a ring structure, for example, but not limited to a 5-carbon, 6-carbon, etc. aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure above where n is 0 to 2 would comprise compound groups including, but not limited to:

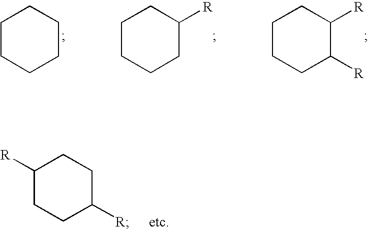

The term “carbonyl” as used herein refers to the —(C═O)— group.

The term “carboxyl” as used herein refers to the —COOH group.

“Dialkylcarbamoyl” as used herein refers to R′RN—CO— group wherein each of R and R′ is independently alkyl as previously described.

The term “halo” is defined as being selected from the group consisting of Br, Cl, I and F.

The term “hydroxyl” as used herein refers to the —OH group.

The term “hydroxyalkyl” as used herein refers to an alkyl group substituted with an —OH group.

The term “furfuryl” as used herein refers to the group comprised of a methyl furan radical.

The term “nitrile” as used herein refers to the —C≡N group.

The term “nitro” is defined as the functional group —NO₂.

The term “oxo” as used herein refers to a compound wherein a carbon atom is replaced by an oxygen atom.

The term “arylsulfanyl” refers to a substituent group having the general formula Ar—S—, wherein Ar is an aryl group as defined herein. One non-limiting example of a compound encompassed by the term “arylsulfanyl” as used herein would be phenylsulfanyl.

The term “arylsulfinyl” refers to a substituent group having the general formula Ar—S(═O)—, wherein Ar is an aryl group as defined herein and S(═O) represents an oxygen atom bound to the sulfur atom through a double bond. One non-limiting example of a compound encompassed by the term “arylsulfinyl” as used herein would be phenylsulfinyl.

The term “arylsulfonyl” refers to a substituent group having the general formula Ar—S(═O)₂—, wherein Ar is an aryl group as defined herein and S(═O)₂ represents two oxygen atoms which are each bound to the sulfur atom through a double bond. One non-limiting example of a compound encompassed by the term “arylsulfonyl” as used herein would be phenylsulfonyl.

The term “sulfonic acid” refers to a compound comprising the functional group R—S(═O)₂OH, wherein R is alkyl or aryl as defined herein and S(═O)₂ represents two oxygen atoms which are each bound to the sulfur atom through a double bond.

The term “thio” as used herein refers to a compound wherein a carbon or oxygen atom is replaced by a sulfur atom.

II. Novel Compounds

A. Representative Embodiments

Described herein is a compound of the formula: 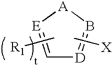
wherein A is selected from the group consisting of oxygen, sulfur, and NR₁₅, and R₁₅ is selected from the group consisting of H, alkyl, aryl, substituted alkyl, and substituted aryl; B,D, and E are each independently selected from the group consisting of CH, nitrogen, sulfur and oxygen; R₁ is selected from the group consisting of nitro, halo, alkyl ester, arylsulfanyl, arylsulfinyl, arylsulfonyl and sulfonic acid; t is an integer from 1 to 3 (e.g., 1, 2 or 3); and X is a substituted amide. In particular embodiments, R₁ is nitro.

In some embodiments, X has the formula: 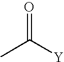
wherein Y is a substituted amine. In particular embodiments, Y is an aromatic monoamine.

In some embodiments, the novel compounds are defined as having a formula as follows: 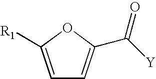
wherein R₁ is selected from the group consisting of halo, nitro, alkyl ester, arylsulfanyl, arylsulfinyl, arylsulfonyl and sulfonic acid; and Y is a substituted amine.

In some embodiments of the novel compounds, Y can be:
(a) —NR₂R₃, wherein R₂ and R₃ are each independently selected from the group consisting of H, alkyl, aryl, substituted alkyl, and substituted aryl, or R₂ and R₃ together form a ring structure with the nitrogen atom to which they are attached; 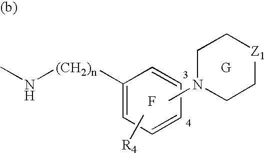
   wherein n is an integer from 0 to 8 (e.g., 0, 1, 2, 3, 4, 5, 6, 7, or 8); R₄ is selected from the group consisting of hydrogen, halo, alkyl, substituted alkyl, and alkoxyl; and Z₁ is selected from the group consisting of oxygen, sulfur, sulfoxide, sulfone, NR₅, and 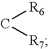
   wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 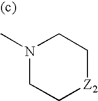
   wherein Z₂ is selected from the group consisting of oxygen, NR₁₀, and 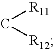
   R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; 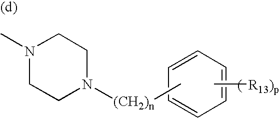
   wherein n is an integer from 0 to 8 (e.g., 0, 1, 2, 3, 4, 5, 6, 7, or 8); p is an integer from 1 to 5 (e.g., 1, 2, 3, 4, or 5); and R₁₃ is selected from the group consisting of hydrogen, alkyl, substituted alkyl, aryl, and substituted aryl; 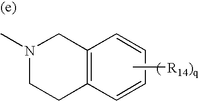
   wherein q is an integer from 1 to 4 (e.g., 1, 2, 3, or 4); and R₁₄ is selected from the group consisting of hydrogen, alkyl, substituted alkyl, aryl, and substituted aryl; 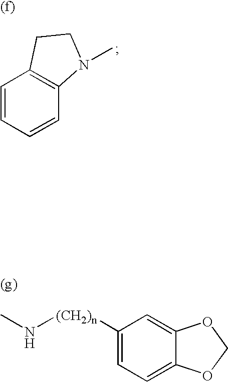
   wherein n is an integer from 0 to 8 (e.g., 0, 1, 2, 3, 4, 5, 6, 7, or 8); or 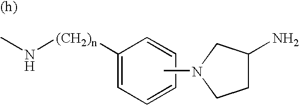
   wherein n is an integer from 0 to 8 (e.g., 0, 1, 2, 3, 4, 5, 6, 7, or 8).

In some embodiments, Y is NR₂R₃ and R₁ is nitro, R₂ is H and R₃ is aryl or substituted aryl. In other embodiments, Y is NR₂R₃ and R₁ is nitro, and R₂ and R₃ together form a ring structure with the nitrogen atom to which they are attached.

In some embodiments, Y is: 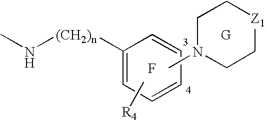
and n can be zero or one. Further, in some embodiments ring G is in the 3-position or 4-position of ring F. In some embodiments, Z₁ is oxygen or sulfur. In other embodiments Z₁ is NR₅. In still other embodiments, Z₁ is

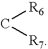

In some embodiments, Y is: 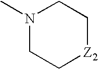
and Z₂ is NR₁₀, wherein R₁₀ is selected from the group consisting of hydrogen, alkyl, substituted alkyl, aryl, and substituted aryl In other embodiments, Z₂ is 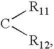
wherein R₁₁ and R₁₂ are each independently selected from the group consisting of hydrogen, alkyl, substituted alkyl, aryl, substituted aryl, and hydroxyl.

In some embodiments, Y is: 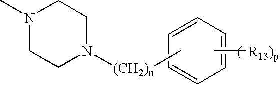
and n is zero or one.

Further, in some embodiments of the novel compounds disclosed herein having the general formula: 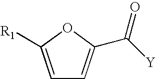
wherein R₁ is selected from the group consisting of halo, nitro, alkyl ester, arylsulfanyl, arylsulfinyl, arylsulfonyl and sulfonic acid; Y can be:
(a) —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached; 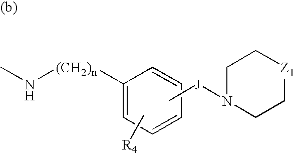
   wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 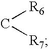
   wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 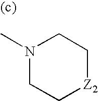
   wherein Z₂ is selected from the group consisting of oxygen, NR₁₀, and 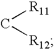
   R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; and 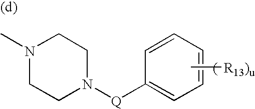
   wherein Q is selected from the group consisting of (CH₂)ₛ and —C(═O)— and s is an integer from 0 to 8; u is an integer from 1 to 5; and R₁₃ is selected from the group consisting of hydrogen, halo, alkyl, substituted alkyl, aryl, and substituted aryl; or
a pharmaceutically acceptable salt thereof.

In some embodiments, Y is: 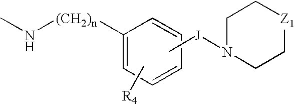
and n is 1 and R₄ is hydrogen. In still other embodiments, J is —(CH₂)_{q}—CH═N— and q is 0. In still further embodiments, Z₁ is NR₅ and R₅ is an alkyl.

In some embodiments, Y is: 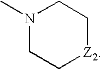
and Z₂ is NR₁₀ . In some embodiments, R₁₀ is a substituted aryl. Further, in some embodiments, R₁₀ is: 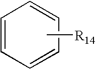
and R₁₄ is selected from the group consisting of halo, alkyl, aryl, substituted alkyl, and substituted aryl.

In some embodiments, Y is: 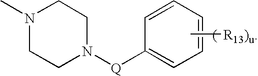
and Q is —C(═O)—. Further, in some embodiments, R₁₃ is selected from the group consisting of a halo an alkoxyl.

In some embodiments of the novel compounds disclosed herein, Y can be 2-anisidine, 3-halo-aniline, 3-anisidine, 4-anisidine, cyclohexylamine, adamantyl amine, furfuryl amine, 4-amino-benzonitrile, 4-methoxy-benzylamine, 2-chloro-benzylamine, 2,4-dimethoxy-benzylamine, 3,4-dimethoxy-benzylamine, 3,4,5-trimethoxy-benzylamine, 1-amino-1,2,3,4-tetrahydro-napththalene, 1-amino-indane, phenethylamine, 4-ethoxy-phenethylamine, (S)-1-phenyl-ethylamine, (R)-1-phenyl-ethylamine, 3,4-dimethoxy-phenethylamine, 4-methoxy-benzylamine, 3-amino-phenol, 3-benzyloxy-aniline, N-methyl-aniline, N-methyl4-anisidine, 2,3-dihydro-indole, 2-amino-pyridine, 3-amino-pyridine, 4-amino-pyridine, 3-amino-pyrazole, 2-amino-pyrazine, 2-amino methyl pyridine, 2-amino-4-methoxy-benzothiazole, 4-amino-6-methoxy-pyrimidine, 2-methoxy-benzylamine, or 2,3-dimethoxy-benzylamine. In particular embodiments, Y is 3-chloro-aniline, 3-fluoro-aniline, 3-anisidine, 4-methoxy-benzylamine, or 3,4-dimethoxy-benzylamine.

Particular compounds of the novel compounds disclosed herein include but are not limited to: 5-nitrofuran-2-carboxylic acid(3-chloro-phenyl)-amide; 5-nitrofuran-2-carboxylic acid(3-bromo-phenyl)-amide; 5-nitrofuran-2-carboxylic acid(3-fluoro-phenyl)-amide; 5-nitrofuran-2-carboxylic acid(4-methoxy-phenyl)-amide; 5-nitrofuran-2-carboxylic acid(3-methoxy-phenyl)-amide; 5-nitrofuran-2-carboxylic acid adamantan-1-ylamide; 5-nitrofuran-2-carboxylic acid phenylamide; 5-nitrofuran-2-carboxylic acid(furan-2-ylmethyl)-amide; 5-nitrofuran-2-carboxylic acid(4-cyano-phenyl )-amide; 5-nitrofuran-2-carboxylic acid 4-methoxy-benzylamide; 5-nitrofuran-2-carboxylic acid 2-chlorobenzylamide; 5-nitrofuran-2-carboxylic acid 2,4-dimethoxybenzylamide; 5-nitrofuran-2-carboxylic acid 3,4-dimethoxybenzylamide; 5-nitrofuran-2-carboxylic acid 3,4,5-trimethoxybenzylamide; 5-nitrofuran-2-carboxylic acid (1,2,3,4-tetrahydro-naphthalen-1-yl)-amide; 5-nitrofuran-2-carboxylic acid indan-1-ylamide; 5-nitrofuran-2-carboxylic acid phenethyl-amide; 5-nitrofuran-2-carboxylic acid [2-(4-methoxy-phenyl )-ethyl]-amide; 5-nitrofuran-2-carboxylic acid(1-phenyl-ethyl )-amide; 5-nitrofuran-2-carboxylic acid [2-(3,4-dimethoxy-phenyl)-ethyl]-amide; 5-nitrofuran-2-carboxylic acid 4-methoxy-benzylamide; 5-nitrofuran-2-carboxylic acid(3-methoxy-phenyl)-amide; 5-sulfo-furan-2-carboxylic acid; 5-(3-methoxy-phenylcarbamoyl)-furan-sulfonic acid; 5-nitrofuran-2-carboxylic acid(3-hydroxy-phenyl)-amide; 5-phenylsulfanyl-furan-2-carboxylic acid(3-methoxy-phenyl)-amide; 5-nitrofuran-2-carboxylic acid(3-benzyloxy-phenyl)-amide; 5-benzenesulfinyl-furan-2-carboxylic acid(3-methoxy-phenyl)-amide; 5-benzenesulfonyl-furan-2-carboxylic acid(3-methoxy-phenyl)-amide; 5-nitrofuran-2-carboxylic acid pyrazin-2-ylamide; 5-nitrofuran-2-carboxylic acid(pyridin-2-yl methyl)-amide; 5-nitrofuran-2-carboxylic acid(4-methoxy-benzothiazol-2-yl)-amide; 5-nitrofuran-2-carboxylic acid(6-methoxy-pyrimin-4-yl)-amide; 5-nitrofuran-2-carboxylic acid 2-methoxy-benzylamide; 5-nitrofuran-2-carboxylic acid 2,3-dimethoxy-benzylamide; 5-nitrofuran-2-carboxylic acid 4-thiomorpholin4-yl-benzylamide; 5-nitrofuran-2-carboxylic acid 4-(1-oxo-114-thiomorpholin4-yl)-benzylamide; 5-nitrofuran-2-carboxylic acid 4-(1,1-dioxo-116-thiomorpholin-4-yl)-benzylamide; 5-nitrofuran-2-carboxylic acid 4-(3-amino-pyrrolidin-1-yl)-benzylamide; 4-(4-{[(5-nitrofuran-2-carbonyl)-amino]-methyl}-phenyl)-piperazine-1-carboxylic acid tert-butyl ester; 5-nitrofuran-2-carboxylic acid 4-piperazin-1-yl-bezylamide; 5-nitrofuran-2-carboxylic acid 4-(4-cyclopropylmethyl-piperazin-1-yl)-benzylamide; 5-nitrofuran-2-carboxylic acid 4-(4-benzyl-piperazin-1-yl)-3-fluoro-benzylamide; 5-nitrofuran-2-carboxylic acid 3-flouro-4-(4-methyl-piperazin-1-yl)-benzylamide; 5-nitrofuran-2-carboxylic acid 3-flouro-4-thiomorpholin-4-yl-benzylamide; 5-nitrofuran-2-carboxylic acid 3-fluoro-4-morpholin-4-yl-benzylamide; 5-nitrofuran-2-carboxylic acid 4-(4-benzyl-piperidin-1-yl)-3-fluoro-benzylamide; 4-(4-{[(5-nitrofuran-2-carbonyl)-amino]-methyl}-phenyl)-piperazine-1-carboxylic acid ethyl ester; 4-(4-{[(5-nitrofuran-2-carbonyl)-amino]-methyl}-phenyl)-piperazine-1-carboxylic acid propylamide; 4-(4-{[(5-nitrofuran-2-carbonyl)-amino]-methyl}-phenyl)-piperazine-1-carboxylic acid isopropylamide; N-(4-methoxybenzyl)-5-nitrofuran-2-carboxamide; (3,4-dihydro-6,7-dimethoxyisoquinolin-2(1H)-yl)(5-nitrofuran-2-yl)methanone; N-((benzo[d][1,3]dioxol-6-yl)methyl)-5-nitrofuran-2-carboxamide; N-(2,4-dimethoxybenzyl)-5-nitrofuran-2-carboxamide; N-(3,4-dimethoxyphenethyl)-5-nitrofuran-2-carboxamide; N-(3,4-dimethoxybenzyl)-5-nitrofuran-2-carboxamide; N-(4-methoxyphenyl)-5-nitrofuran-2-carboxamide; [4-(4-Methoxy-phenyl)-piperazin-1-yl]-(5-nitro-furan-2-yl)-methanone; [4-(4-Fluoro-phenyl)-piperazin-1-yl]-(5-nitro-furan-2-yl)-methanone; [4-(4-Fluoro-benzoyl)-piperazin-1-yl]-(5-nitro-furan-2-yl)-methanone; [4-(4-Methoxy-benzoyl)-piperazin-1-yl]-(5-nitro-furan-2-yl)-methanone; and 5-Nitro-furan-2-carboxylic acid 4-[(4-methyl-piperazin-1-ylimino)-methyl]-benzylamide.

B. Prodruqs

In representative embodiments, compounds disclosed herein are prodrugs. A prodrug means a compound that, upon administration to a recipient, is capable of providing (directly or indirectly) a compound of this invention or an inhibitorily active metabolite or residue thereof. Prodrugs can increase the bioavailability of the compounds of the presently disclosed subject matter when such compounds are administered to a subject (e.g., by allowing an orally administered compound to be more readily absorbed into the blood) or can enhance delivery of the parent compound to a biological compartment (e.g., the brain or lymphatic system) relative to a metabolite species, for example.

C. Pharmaceutically Acceptable Salts

Additionally, the active compounds can be administered as pharmaceutically acceptable salts. Such salts include the gluconate, lactate, acetate, tartarate, citrate, phosphate, borate, nitrate, sulfate, and hydrochloride salts. The salts of the compounds described herein can be prepared, in general, by reacting one equivalent of the base compound with the desired acid, in solution. After the reaction is complete, the salts are crystallized from solution by the addition of an appropriate amount of solvent in which the salt is insoluble.

III. Methods of Utilizing Novel Compounds

The novel compounds disclosed herein have utility in killing or inhibiting the growth of microorganisms and in the treatment of subjects infected with microorganisms. As such, disclosed herein below are methods of killing or inhibiting the growth a microorganism comprising contacting the microorganism with an effective amount of a novel compound disclosed herein. Also disclosed herein below are methods of treating a microbial infection in a subject comprising administering to the subject a therapeutically effective amount of a novel compound disclosed herein.

Microorganisms killed or growth-inhibited and microbial infections treated by the novel compounds and methods disclosed herein include a variety of microbes, including fungi, algae, protozoa, bacteria, and viruses. Exemplary microorganisms killed or growth-inhibited and microbial infections that can be treated by the methods of the presently disclosed subject matter include, but are not limited to, infections caused by bacteria, specifically members of the genus Mycobacterium. As a non-limiting example, the members can include Mycobacterium tuberculosis, which can cause the disease tuberculosis, in all its forms, in animal subjects.

The methods disclosed herein are useful for treating these conditions in that they inhibit the onset, growth, or spread of the condition, cause regression of the condition, cure the condition, or otherwise improve the general well-being of a subject afflicted with, or at risk of contracting the condition.

Methods of killing or inhibiting the growth of a microorganism or treating a microbial infection comprise contacting the microorganism with, or administering to a subject in need of treatment, respectively, an active compound as described herein. These active compounds, as set forth above, include the compounds, their corresponding prodrugs, and pharmaceutically acceptable salts of the compounds and prodrugs.

With regard to the presently described method embodiments, representative compounds can have a structure as follows: 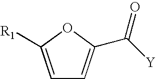
wherein R₁ is selected from the group consisting of halo, nitro, alkyl ester, arylsulfanyl, arylsulfinyl, arylsulfonyl and sulfonic acid; and Y is a substituted amine.

In some embodiments of the novel compounds, Y can be:
(a) —NR₂R₃, wherein R₂ and R₃ are each independently selected from the group consisting of H, alkyl, aryl, substituted alkyl, and substituted aryl, or R₂ and R₃ together form a ring structure with the nitrogen atom to which they are attached; 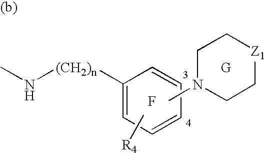
   wherein n is an integer from 0 to 8; R₄ is selected from the group consisting of hydrogen, halo, alkyl, substituted alkyl, and alkoxyl; and Z₁ is selected from the group consisting of oxygen, sulfur, sulfoxide, sulfone, NR₅, and 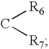
   wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 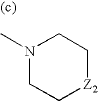
   wherein Z₂ is selected from the group consisting of oxygen, NR₁₀, and 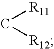
   R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; 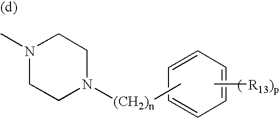
   wherein n is an integer from 0 to 8; p is an integer from 1 to 5; and R₁₃ is selected from the group consisting of hydrogen, alkyl, substituted alkyl, aryl, and substituted aryl; 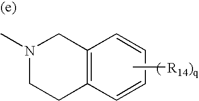
   wherein q is an integer from 1 to 4; and R₁₄ is selected from the group consisting of hydrogen, alkyl, substituted alkyl, aryl, and substituted aryl; 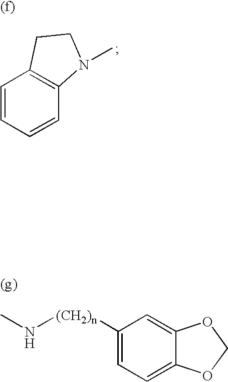
   wherein n is an integer from 0 to 8; or 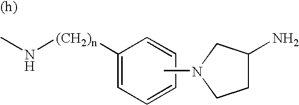
   wherein n is an integer from 0 to 8.

Further, in some embodiments of the novel compounds disclosed herein having the general formula: 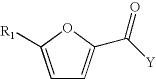
wherein R₁ is selected from the group consisting of halo, nitro, alkyl ester, arylsulfanyl, arylsulfinyl, arylsulfonyl and sulfonic acid; Y can be:
(a) —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached; 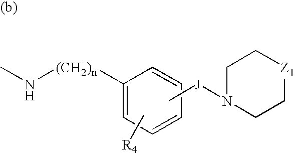
   wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 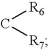
   wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 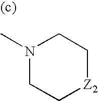
   wherein Z₂ is selected from the group consisting of oxygen, NR₁₀, and 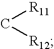
   R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; and 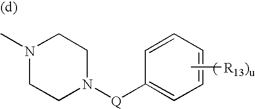
   wherein Q is selected from the group consisting of (CH₂)ₛ and —C(═O)— and s is an integer from 0 to 8; u is an integer from 1 to 5; and R₁₃ is selected from the group consisting of hydrogen, halo, alkyl, substituted alkyl, aryl, and substituted aryl; or
a pharmaceutically acceptable salt thereof.

In some embodiments, Y is: 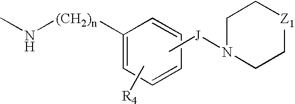
and n is 1 and R₄ is hydrogen. In still other embodiments, J is —(CH₂)_{q}—CH═N— and q is 0. In still further embodiments, Z₁ is NR₅ and R₅ is an alkyl.

In some embodiments, Y is: 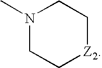
and Z₂ is NR₁₀. In some embodiments, R₁₀ is a substituted aryl. Further, in some embodiments, R₁₀ is: 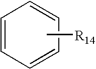
and R₁₄ is selected from the group consisting of halo, alkyl, aryl, substituted alkyl, and substituted aryl.

In some embodiments, Y is: 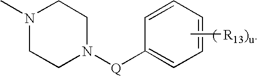
and Q is —C(═O)—. Further, in some embodiments, R₁₃ is selected from the group consisting of a halo an alkoxyl.

In some embodiments of the novel compounds disclosed herein, Y can be anisidine, 3-halo-aniline, 3-anisidine, 4-anisidine, cyclohexylamine, adamantyl amine, furfuryl amine, 4-amino-benzonitrile, 4-methoxy-benzylamine, 2-chloro-benzylamine, 2,4-dimethoxy-benzylamine, 3,4-dimethoxy-benzylamine, 3,4,5-trimethoxy-benzylamine, 1-amino-1,2,3,4-tetrahydro-napththalene, 1-amino-indane, phenethylamine, 4-ethoxy-phenethylamine, (S)-1-phenyl-ethylamine, (R)-1-phenyl-ethylamine, 3,4-dimethoxy-phenethylamine, 4-methoxy-benzylamine, 3-amino-phenol, 3-benzyloxy-aniline, N-methyl-aniline, N-methyl-4-anisidine, 2,3-dihydro-indole, 2-amino-pyridine, 3-amino-pyridine, 4-amino-pyridine, 3-amino-pyrazole, 2-amino-pyrazine, 2-amino methyl pyridine, 2-amino-4-methoxy-benzothiazole, 4-amino-6-methoxy-pyrimidine, 2-methoxy-benzylamine, or 2,3-dimethoxy-benzylamine. In particular embodiments, Y is 3-chloro-aniline, 3-fluoro-aniline, 3-anisidine, 4-methoxy-benzylamine, or 3,4-dimethoxy-benzylamine.

Particular compounds of the novel compounds disclosed herein include but are not limited to: 5-nitrofuran-2-carboxylic acid(3-chloro-phenyl)-amide; 5-nitrofuran-2-carboxylic acid(3-bromo-phenyl)-amide; 5-nitrofuran-2-carboxylic acid(3-fluoro-phenyl)-amide; 5-nitrofuran-2-carboxylic acid(4-methoxy-phenyl)-amide; 5-nitrofuran-2-carboxylic acid(3-methoxy-phenyl)-amide; 5-nitrofuran-2-carboxylic acid adamantan-1-ylamide; 5-nitrofuran-2-carboxylic acid phenylamide; 5-nitrofuran-2-carboxylic acid(furan-2-ylmethyl)-amide; 5-nitrofuran-2-carboxylic acid(4-cyano-phenyl)-amide; 5-nitrofuran-2-carboxylic acid 4-methoxy-benzylamide; 5-nitrofuran-2-carboxylic acid 2-chlorobenzylamide; 5-nitrofuran-2-carboxylic acid 2,4-dimethoxybenzylamide; 5-nitrofuran-2-carboxylic acid 3,4-dimethoxybenzylamide; 5-nitrofuran-2-carboxylic acid 3,4,5-trimethoxybenzylamide; 5-nitrofuran-2-carboxylic acid (1,2,3,4-tetrahydro-naphthalen-1-yl)-amide; 5-nitrofuran-2-carboxylic acid indan-1-ylamide; 5-nitrofuran-2-carboxylic acid phenethyl-amide; 5-nitrofuran-2-carboxylic acid [2-(4-methoxy-phenyl)-ethyl]-amide; 5-nitrofuran-2-carboxylic acid(1-phenyl-ethyl)-amide; 5-nitrofuran-2-carboxylic acid [2-(3,4-dimethoxy-phenyl)-ethyl]-amide; 5-nitrofuran-2-carboxylic acid 4-methoxy-benzylamide; 5-nitrofuran-2-carboxylic acid(3-methoxy-phenyl)-amide; 5-sulfo-furan-2-carboxylic acid; 5-(3-methoxy-phenylcarbamoyl)-furan-sulfonic acid; 5-nitrofuran-2-carboxylic acid(3-hydroxy-phenyl)-amide; 5-phenylsulfanyl-furan-2-carboxylic acid(3-methoxy-phenyl)-amide; 5-nitrofuran-2-carboxylic acid(3-benzyloxy-phenyl)-amide; 5-benzenesulfinyl-furan-2-carboxylic acid(3-methoxy-phenyl)-amide; 5-benzenesulfonyl-furan-2-carboxylic acid(3-methoxy-phenyl)-amide; 5-nitrofuran-2-carboxylic acid pyrazin-2-ylamide; 5-nitrofuran-2-carboxylic acid(pyridin-2-yl methyl)-amide; 5-nitrofuran-2-carboxylic acid(4-methoxy-benzothiazol-2-yl)-amide; 5-nitrofuran-2-carboxylic acid(6-methoxy-pyrimin-4-yl)-amide; 5-nitrofuran-2-carboxylic acid 2-methoxy-benzylamide; 5-nitrofuran-2-carboxylic acid 2,3-dimethoxy-benzylamide; 5-nitrofuran-2-carboxylic acid 4-thiomorpholin-4-yl-benzylamide; 5-nitrofuran-2-carboxylic acid 4-(1-oxo-114-thiomorpholin-4-yl)-benzylamide; 5-nitrofuran-2-carboxylic acid 4-(1,1-dioxo-116-thiomorpholin-4-yl)-benzylamide; 5-nitrofuran-2-carboxylic acid 4-(3-amino-pyrrolidin-1-yl)-benzylamide; 4-(4-{[(5-nitrofuran-2-carbonyl)-amino]-methyl}-phenyl)-piperazine-1-carboxylic acid tert-butyl ester; 5-nitrofuran-2-carboxylic acid 4-piperazin-1-yl-bezylamide; 5-nitrofuran-2-carboxylic acid 4-(4-cyclopropylmethyl-piperazin-1-yl)-benzylamide; 5-nitrofuran-2-carboxylic acid 4-(4-benzyl-piperazin-1-yl)-3-fluoro-benzylamide; 5-nitrofuran-2-carboxylic acid 3-fluoro-4-(4-methyl-piperazin-1-yl)-benzylamide; 5-nitrofuran-2-carboxylic acid 3-fluoro-4-thiomorpholin-4-yl-benzylamide; 5-nitrofuran-2-carboxylic acid 3-fluoro-4-morpholin-4-yl-benzylamide; 5-nitrofuran-2-carboxylic acid 4-(4-benzyl-piperidin-1-yl)-3-fluoro-benzylamide; 4-(4-{[(5-nitrofuran-2-carbonyl)-amino]-methyl}-phenyl)-piperazine-1-carboxylic acid ethyl ester; 4-(4-{[(5-nitrofuran-2-carbonyl)-amino]-methyl}-phenyl)-piperazine-1-carboxylic acid propylamide; 4-(4-{[(5-nitrofuran-2-carbonyl)-amino]-methyl}-phenyl)-piperazine-1-carboxylic acid isopropylamide; N-(4-methoxybenzyl)-5-nitrofuran-2-carboxamide; (3,4-dihydro-6,7-dimethoxyisoquinolin-2(1H)-yl)(5-nitrofuran-2-yl)methanone; N-((benzo[d][1,3]dioxol-6-yl)methyl)-5-nitrofuran-2-carboxamide; N-(2,4-dimethoxybenzyl)-5-nitrofuran-2-carboxamide; N-(3,4-dimethoxyphenethyl)-5-nitrofuran-2-carboxamide; N-(3,4-dimethoxybenzyl)-5-nitrofuran-2-carboxamide; N-(4-methoxyphenyl)-5-nitrofuran-2-carboxamide; [4-(4-Methoxy-phenyl)-piperazin-1-yl]-(5-nitro-furan-2-yl)-methanone; [4-(4-Fluoro-phenyl)-piperazin-1-yl]-(5-nitro-furan-2-yl)-methanone; [4-(4-Fluoro-benzoyl)-piperazin-1-yl]-(5-nitro-furan-2-yl)-methanone; [4-(4-Methoxy-benzoyl)-piperazin-1-yl]-(5-nitro-furan-2-yl)-methanone; and 5-Nitro-furan-2-carboxylic acid 4-[(4-methyl-piperazin-1-ylimino)-methyl]-benzylamide.

An effective amount of any specific active compound, the use of which is in the scope of embodiments described herein, will vary somewhat from compound to compound, use to use (for example, specifically killing or inhibiting the growth of a microorganism or treating a microbial infection in a subject), and subject to subject when utilizing methods of treating subjects, and will depend upon the condition of the patient and the route of delivery.

As a general proposition, an effective amount is from about 1 to about 1000 micrograms per milliliter (μg/mL) of the compound. In some embodiments, the effective amount is from about 10 to 500 μg/mL. In other embodiments, the effective amount is from about 50 to 250 μg/mL. In some particular embodiments, the effective amount is from about 100 to 200 μg/mL.

The subject treated in the presently disclosed subject matter in its many embodiments is desirably a human subject, although it is to be understood the methods described herein are effective with respect to all vertebrate species, which are intended to be included in the term “subject”. The methods described herein are particularly useful in the treatment and/or prevention of microbial infections in warm-blooded vertebrates. Thus, the methods can be used as treatment for mammals and birds.

More particularly, provided is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, embodiments of the methods described herein include the treatment of livestock, including, but not limited to, domesticated swine (pigs and hogs), ruminants, horses, poultry, and the like.

IV. Pharmaceutical Formulations

The novel compounds disclosed herein, the pharmaceutically acceptable salts thereof, prodrugs corresponding to the novel compounds disclosed herein, and the pharmaceutically acceptable salts thereof, are all referred to herein as “active compounds.” Pharmaceutical formulations comprising the aforementioned active compounds are also provided herein. These pharmaceutical formulations comprise active compounds as described herein, in a pharmaceutically acceptable carrier. Pharmaceutical formulations can be prepared for oral, intravenous, or aerosol administration as discussed in greater detail below. Also, the present invention provides such active compounds that have been lyophilized and that can be reconstituted to form pharmaceutically acceptable formulations for administration, as by intravenous or intramuscular injection.

The therapeutically effective dosage of any specific active compound, the use of which is in the scope of embodiments described herein, will vary somewhat from compound to compound, and patient to patient, and will depend upon the condition of the patient and the route of delivery. As a general proposition, a dosage from about 1 to about 1000 μg/mL of the compound within the formulation is considered an effective dosage. In some embodiments, the effective amount is from about 10 to 500 μg/mL. In other embodiments, the effective amount is from about 50 to 250 μg/mL. In some particular embodiments, the effective amount is from about 100 to 200 μg/mL.

In accordance with the present methods, pharmaceutically active compounds as described herein can be administered orally as a solid or as a liquid, or can be administered intramuscularly or intravenously as a solution, suspension, or emulsion. Alternatively, the compounds or salts can also be administered by inhalation, intravenously or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical formulations suitable for intravenous or intramuscular injection are further embodiments provided herein. The pharmaceutical formulations comprise a compound of Formula I described herein, a prodrug as described herein, or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. Of course, the dispensing is preferably done by an aseptic method. Sterilized closures can then be placed on the vials and, if desired, the vial contents can be lyophilized.

In addition to the novel compounds disclosed herein, or their salts or prodrugs, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain anti-microbial preservatives. Useful anti-microbial preservatives include methylparaben, propylparaben, and benzyl alcohol. The anti-microbial preservative is typically employed when the formulation is placed in a vial designed for multi-dose use. The pharmaceutical formulations described herein can be lyophilized using techniques well known in the art.

In yet another aspect of the subject matter described herein, there is provided an injectable, stable, sterile formulation comprising a novel compound disclosed herein, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound or salt. When the compound or salt is substantially water-insoluble, a sufficient amount of emulsifying agent, which is physiologically acceptable, can be employed in sufficient quantity to emulsify the compound or salt in an aqueous carrier. One such useful emulsifying agent is phosphatidyl choline.

Other pharmaceutical formulations can be prepared from the water-insoluble compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines, and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations containing the active compounds disclosed herein can be lyophilized to produce a lyophilizate, which can be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used in the present specification, the term “water-soluble” is meant to define any composition that is soluble in water in an amount of about 50 mg/mL, or greater. Also, as used in the present specification, the term “water-insoluble” is meant to define any composition that has solubility in water of less than about 20 mg/mL. For certain applications, water-soluble compounds or salts can be desirable whereas for other applications water-insoluble compounds or salts likewise can be desirable.

### EXAMPLES

The following Examples have been included to illustrate modes of the presently disclosed subject matter. Certain aspects of the following Examples are described in terms of techniques and procedures found or contemplated to work well in the practice of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

Galactofuranose is an essential component of the mycobacterial cell wall and not found in humans, UDP-galactofuranose is biosynthesized from UDP-galactopyranose using the enzyme UDP-galactose mutase (Glf). Disclosed herein is a microtitre plate based screen of Glf used to discover novel inhibitors as potential new anti-tuberculosis agents. In the course of using the screen a nitrofuranylamide was discovered to be an inhibitor of Glf with an IC₅₀ of 7 μg/mL. Noticeably, this compound had good activity against whole cells with an MIC of 1.6 μg/mL. Example 1 describes efforts at developing the structure activity relationship of the nitrofuranylamides with respect to Glf inhibition and anti-tuberculosis activity, as well as deriving other even more effective compounds having anti-tuberculosis activity.

### Methods and Materials

All the anhydrous solvents and starting materials were purchased from Aldrich Chemical Company (Milwaukee, Wis., U.S.A.). All reagent grade solvents used for chromatography were purchased from Fisher Scientific (Suwanee, Ga., U.S.A.) and FLASH™ column chromatography silica cartridges were obtained from Biotage Inc. (Lake Forest, Va., U.S.A.). The reactions were monitored by thin layer chromatography (TLC) on pre-coated Merck 60 F₂₅₄ silica gel plates and visualized using UV light (254 nm). Biotage FLASH 25+™ column chromatography system was used to purify mixtures. All ¹H and ¹³C NMR spectra were recorded on a Bruker ARX-300 (300 and 75 MHz for ¹H and ¹³C NMR₁ respectively; Billerica, Mass., U.S.A.) or Varian INOVA-500™ (500 and 125 MHz for ¹H and ¹³C NMR₁ respectively; Palo Alto, Calif., U.S.A.) spectrometer. Chemical shifts are reported in ppm (δ) relative to residual solvent peak or internal standard (tetramethylsilane) and coupling constants (J) are reported in hertz (Hz). Mass spectra were recorded on a Bruker ESQUIRE LCMS™ using ESI. Purity of the final products was confirmed before testing by analytical HPLC using a Alltech (Deerfield, Ill., U.S.A.) platinum C-18 reverse phase column (4.5×I50 mm) and a H₂O (0.1% TFA) to acetonitrile 0-100% linear gradient at a flow rate of 1.0 mL min⁻¹ and UV detection at 254 nm.

### Scheme 1

Referring now to Scheme 1, the synthesis of nitrofuranyl amides 10-31 and 35 were prepared by reacting the corresponding amines with 5-nitro-2-furan carboxylic acid 5a in presence of EDCl (Scheme 1) according to the parallel synthesis protocol of Boger. This route was later replaced when coupling less reactive heteroaromatic amines by using acid chloride chemistry 6a to form amides 41-53. This route has proved to be more cost effective and scalable.

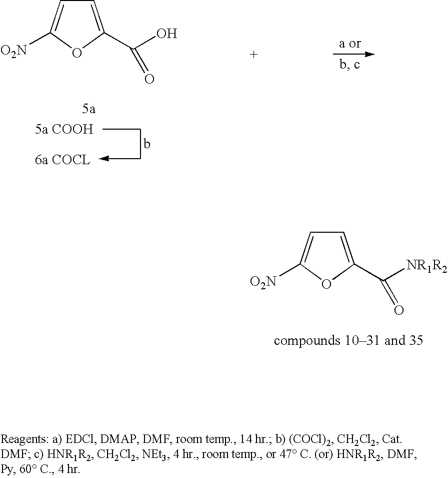

### Scheme 2

Referring now to Scheme 2, nitrofuranyl amide 35 was further elaborated into amide 37 by benzylating the phenolic hydroxyl group using standard benzylation conditions.

In order to evaluate the importance of the nitro functionality on 5-position of furan ring other furanyl amides were synthesized. Accordingly, the 5-bromofuranyl amides 32, and 33 were synthesized by reacting 5-bromo furan carboxylic acid with corresponding amines in presence of EDCl (Scheme 2). The 5-bromo substitution on furan ring was subsequently used to effect further transformations at the 5-position. Bromofuranyl amide 33 was converted to thioether 36 by a nucleophilic substitution of the bromine on furan ring with thiophenol. The thioether 36 upon oxidation provided sulfoxide and sulfone amides 38 and 39 (Scheme 2).

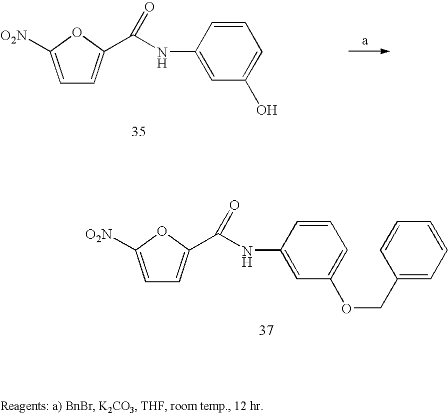

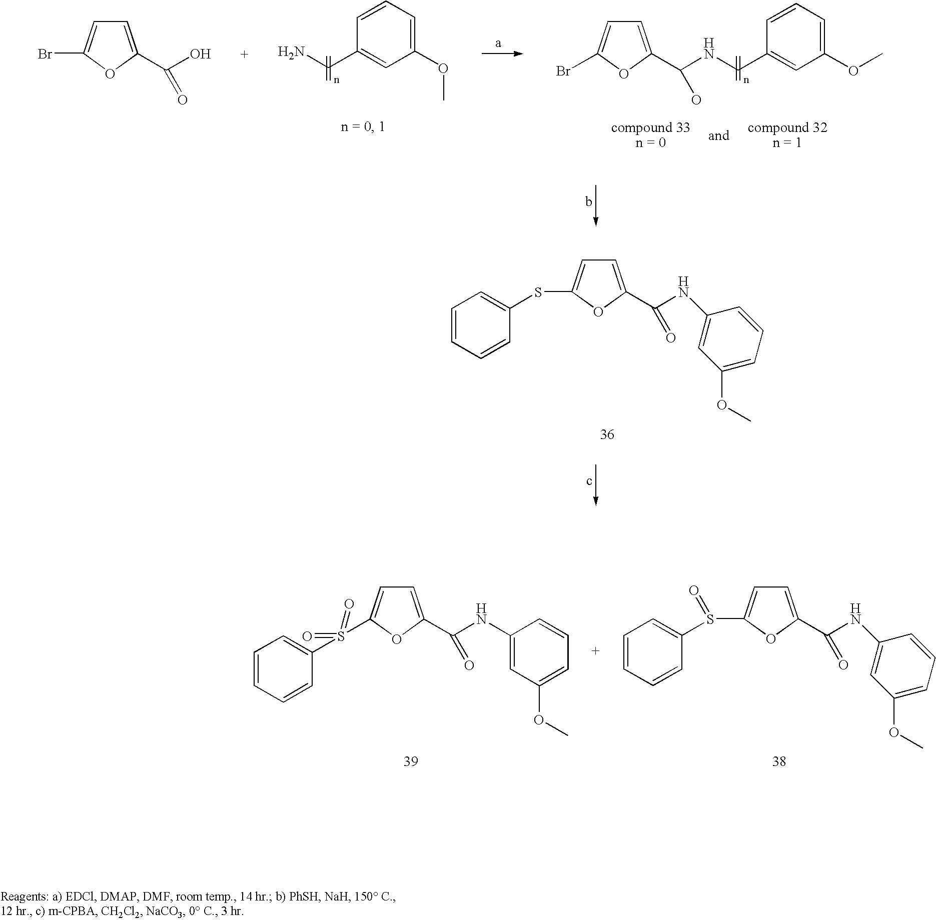

### Scheme 3

Referring now to Scheme 3, furanylamide bromide 33 was also converted to furanylamide ester 40 using standard Grignard chemistry (Scheme 3). Treatment of the bromide with ethyl magnesium bromide followed by reaction of the intermediate with ethylchloroformate afforded the target ester 40.

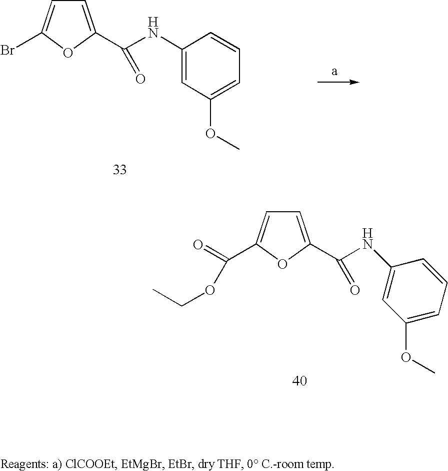

### Scheme 4

Referring now to Scheme 4, the sulfonate analog 34 was synthesized from commercially available aldehyde 8a, which was oxidized to acid 9a with silver nitrate and coupled to m-anisidine using the standard EDCl mediated coupling protocol to yield amide 34 (Scheme 4).

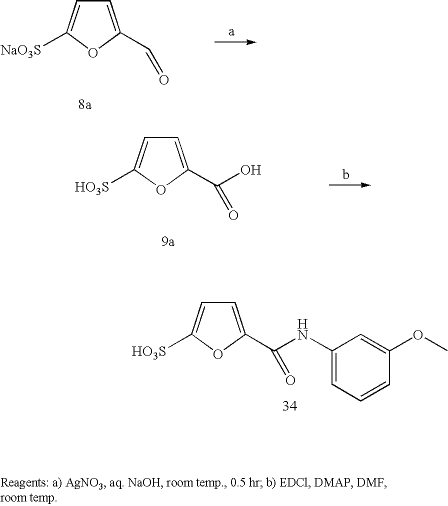

General procedure for preparation of amides 10-30, 32, 33 and 35. 5-Nitro-2-furan carboxylic acid (300 mg, 1.9 mmol, 1 equiv) and amine (1.9 mmol, 1 equiv) in DMF (5 mL) was treated with EDCl (730 mg, 3.8 mmol, 2 equiv) followed by DMAP (582 mg, 4.7 mmol, 2.5 equiv) and the resulting solution was stirred for 14 hr. at 25° C. The reaction mixture was poured into EtOAc (75 mL) and washed with 10% aqueous HCl (2×50 mL) and washed with 10% aqueous NaHCO₃ (3×50 mL). The organic phase was dried (Na₂SO₄), filtered, and concentrated followed by flash-column purification with Pet. Ether and EtOAc system provided corresponding amides (yields generally wary from 69% to 92%).

5-Nitro-furan-2-carboxylic acid(3-chloro-phenyl)-amide (10). 5-Nitro-2-furan carboxylic acid (300 mg, 1.9 mmol) and m-chloro aniline (202 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 432 mg product (85% yield). TLC: R_{f} 0.82 (1:1 hexane:ethyl acetate); ¹H NMR (300 MHz, CDCl₃): δ7.23 (1H, ddd, J=7.8 Hz, 2.0 Hz, 1.0 Hz), 7.35 (1H, t, J=7.8 Hz), 7.44 (2H, q, J=9.0 Hz, 3.8 Hz), 7.54 (1H, ddd, J=7.8 Hz, 2.0 Hz, 1.0 Hz), 7.84 (1H, t, J=2.0 Hz), 8.27-8.33 (1H, bs); ¹³C NMR (300 MHz, CDCl₃): 112.09, 116.58, 117.85, 116.58, 120.02, 125.10, 129.70, 134.45, 136.93, 146.88, 153.45; EI-Mass: 265 (M⁺−1).

5-Nitro-furan-2-carboxylic acid(3-bromo-phenyl)-amide (11). 5-Nitro-2-furan carboxylic acid (300 mg, 1.9 mmol) and m-bromo aniline (306 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 469 mg product (79% yield). TLC: R_{f} 0.82 (1:1 hexane:ethyl acetate); H NMR (300 MHz, CDCl₃): δ7.28 (1H, t, J=7.7 Hz), 7.36 (1H, t, J=1.4 Hz), 7.43 (2H, q, T=9.6 Hz, 3.8 Hz), 7.6 (1H, ddd, J=7.7 Hz, 2.1 Hz, 1.2 Hz), 7.98 (1H, t, J 2.1 Hz), 8.23-8.3 (1H, bs); ¹³C NMR (300 MHz, CDCl₃): 112.05, 116.54, 118.39, 122.87, 127.95, 129.95, 137.20, 140.72, 146.94, 153.50, 158.97; EI-Mass: 310.8 (M⁺−1).

5-Nitro-furan-2-carboxylic acid(3-fluoro-phenyl)-amide (12). 5-Nitro-2-furan carboxylic acid (300 mg, 1.9 mmol) and m-fluoro aniline (184 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 429 mg product (89% yield). R_{f} 0.82 (1:1 hexane:ethyl acetate); ¹H NMR (300 MHz, CDCl₃): δ6.9-6.98 (1H, m), 7.33-7.4 (2Hs, m), 7.44 (2Hs, q, J=8.8 Hz, 3.8 Hz), 7.64-7.7 (1H, m), 8.3-8.4 (1H, bs); ¹³C NMR (300 MHz, CDCl₃): 164.07, 160.81, 153.44, 146.91, 137.35, 129.93, 116.55, 115.16, 112.09, 107.64; EI-Mass: 248.8 (M⁺−1).

5-Nitro-furan-2-carboxylic acid(3-methoxy-phenyl)-amide (13). 5-Nitro-2-furan carboxylic acid (300 mg, 1.9 mmol) and m-anisidine (214 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 450 mg of product (90% yield). TLC: R_{f} 0.75 (1:1 hexane:ethyl acetate); ¹H NMR (300 MHz, CDCl₃): δ6.79 (1H, ddd, J=8.4 Hz, 2.8 Hz, 1.2 Hz), 7.19 (1H, ddd, J=8.4 Hz, 2.16 Hz, 0.7 Hz), 7.32 (1H, t, J=8.4 Hz), 7.39-7.45 (2Hs, m), 8.22-8.28 (1H, bs); ¹³C NMR (300 MHz, CDCl₃): 54.83, 105.65, 110.83, 112.06, 112.13, 116.20, 129.39, 137.06, 147.35, 153.48, 159.71; EI-Mass: 260.8. (M⁺−1).

5-Nitro-furan-2-carboxylic acid(4-methoxy-phenyl)-amide (14). 5-Nitro-2-furan carboxylic acid (300 mg, 1.9 mmol) and p-anisidine (234 mg, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 425 mg of product (85% yield). TLC: R_{f} 0.7 (1:1 hexane:ethyl acetate); ¹H NMR (300 MHz, CDCl₃): δ6.95 (1H, d, J=8.9 Hz), 7.38 (1H, d, J=3.9 Hz), 7.44 (1H, d, J=3.9 Hz), 7.6 (1H, d, J=8.9 Hz), 8.15-8.21 (1H, bs); ¹³C NMR (300 MHz, CDCl₃): 112.11, 113.88, 115.94, 121.67, 128.81, 147.55, 153.29, 156.78, 157.1; EI-Mass: 260.9 (M⁺−1).

5-Nitro-furan-2-carboxylic acid cyclohexylamide (15). 5-Nitro-2-furan carboxylic acid (300 mg, 1.9 mmol) and cyclohexylamine (217 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 322 mg of product (71% yield). TLC: R_{f} 0.72 (1:1 hexane:ethyl acetate); ¹H NMR (300 MHz, CDCl₃): δ1.15-1.5 (6Hs, m), 1.82 (2Hs, dt, J=9.7 Hz, 2.9 Hz), 2.03 (2 Hs, dd, J=12.3 Hz, 2.6), 3.97 (1H, m), 6.42 (1H, bd, J=6.2 Hz), 7.26 (1H, d, J=3.8 Hz), 7.38 (1H, d, J=3.8 Hz); ¹³C NMR (300 MHz, CDCl₃): 23.25, 32.44, 33.06, 50.96, 111.97, 115.10, 147.85, 115.28; EI-Mass: 261.1 (M⁺+23).

5-Nitro-furan-2-carboxylic acid adamantan-1-ylamide (16). 5-Nitro-2-furan carboxylic acid (300 mg, 1.9 mmol) and adamantylamine (288 mg, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 382 mg of product (69% yield). TLC: R_{f} 0.72 (1:1 hexane:ethyl acetate); ¹H NMR (300 MHz, CDCl₃,): δ1.70 (6Hs, s), 2.13 (9Hs, s), 6.18-6.25 (1H, bs), 7.2 (1H, d, J=3.7 Hz), 7.35 (1H, d, J=3.7 Hz); ¹³C NMR (300 MHz, CDCl₃): 28.88, 35.65, 40.95, 52.62, 111.97, 114.78, 148.38, 154.58; EI-Mass: 288.9 (M⁺−1).

5-Nitro-furan-2-carboxylic acid phenylamide (17). 5-Nitro-2-furan carboxylic acid (300 mg, 1.9 mmol) and aniline (152 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 376 mg of product (85% yield). TLC: R_{f} 0.75 (1:1 hexane:ethyl acetate); ¹H NMR (300 MHz, CDCl₃): δ7.24 (1H, tt, J=7.9 Hz, 0.8 Hz), 7.39-7.48 (4Hs, m), 7.7 (2H, dd, J=8.4 Hz, 0.8 Hz), 8.22-8.28 (1K, bs); ¹³C NMR (300 MHz, CDCl₃): 112.10, 116.21, 119.90, 125.03, 128.73, 135.83, 147.34, 153.42; EI-Mass: 230.8 (M⁺−1).

5-Nitro-furan-2-carboxylic acid(furan-2-ylmethyl)-amide (18). 5-Nitro-2-furan carboxylic acid (300 mg, 1.9 mmol) and 2-aminomethyl furan (92 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 383 mg of product (85% yield). TLC: R_{f} 0.68 (1:1 hexane:ethyl acetate); ¹H NMR (300 MHz, CDCl₃): δ4:67 (2Hs, d, J=5.1 Hz, 3.0 Hz, 2.0 Hz, 0.4 Hz), 6.33-6.4 (2H, m), 6.86-6.94 (1H, bs), 7.32 (1H, d, J=3.9 Hz), 7.38 (1H, d, J=3.9 Hz), 7.42 (1H, dd, J=2.06 Hz, 0.4 Hz); ¹³C NMR (300 MHz, CDCl₃): 35.82, 107.83, 110.04, 111.81, 115.69, 142.14, 147.23, 149.36, 155.48, 160.59; EI-Mass: 234.8 (M⁺−1);

5-Nitro-furan-2-carboxylic acid(4-cyano-phenyl)-amide (19). 5-Nitro-2-furan carboxylic acid (300 mg, 1.9 mmol) and 4-amino benzonitrile (225 mg, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 441 mg of product (90% yield). TLC: R_{f}. 0.62 (1:1 hexane:ethyl acetate); ¹H NMR (300 MHz, CDCl₃): δ7.52 (1H, d, J=3.9 Hz), 7.61 (1H, d, J=3.9 Hz), 7.76 (1H, d, J 8.9 Hz), 7.98 (1H, d, J=8.9 Hz); ¹³C NMR (300 MHz, CDCl₃): 106.23, 113.29, 117.24, 118.74, 120.51, 133.13, 142.09, 147.17, 151.84, 154.89; EI-Mass: 255.8 (M⁺−1).

5-Nitro-furan-2-carboxylic acid 4-methoxy-benzylamide (20). 5-Nitro-2-furan carboxylic acid (300 mg, 1.9 mmol) and 4-methoxy benzylamine (248 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 448 mg of product (85% yield). TLC: R_{f} 0.55 (1:1 hexane:ethyl acetate); ¹H NMR (300 MHz, CDCl₃): δ3.83 (3Hs, s), 4.58 (2Hs, d, J=5.8 Hz), 6.82-6.92 (1H, bs), 6.92 (2Hs, d, J=8 Hz), 7.27-7.32 (3Hs, m), 7.38 (1H, d, J=3.5 Hz); ¹³C NMR (300 MHz, CDCl₃): 42.57, 54.78, 111.87, 113.73, 115.49, 128.56, 128.92, 147.51, 155.56, 158.85; EI-Mass: 275.6 (M⁺−1).

5-Nitro-furan-2-carboxylic acid 2-chloro-benzylamide (21). 5-Nitro-2-furan carboxylic acid (300 mg, 1.9 mmol) and 2-chlorbenzylamine (230 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 454 mg product (85% yield). TLC: R_{f} 0.72 (1:1 hexane:ethyl acetate); ¹H NMR (300 MHz, CDCl₃): δ4.75 (2Hs, d, J=6.2 Hz), 7.68-7.09 (1H, bs), 7.27-7.32 (3Hs, m), 7.38 (1H, d, J=3.9 Hz), 7.41-7.49 (2Hs, m); ¹³C NMR (300 MHz, CDCl₃): 40.99, 111.82, 115.64, 126.69, 128.91, 129.21, 129.87, 133.27, 133.90, 147.28, 155.67; EI-Mass: 278.8 (M⁺−1).

5-Nitro-furan-2-carboxylic acid 2,4-dimetboxy-benzylamide (22). 5-Nitro-2-furan carboxylic acid (300 mg, 1.9 mmol) and 2,4-dimethoxy-benzylamine (286 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 508 mg of product (87% yield). TLC: R_{f} 0.50 (1:1 hexane:ethyl acetate); ¹H NMR (300 MHz, CDCl₃): δ3.83 (3Hs, s), 3.91 (3Hs, s), 4.57 (2Hs, d, J=5.8 Hz), 6.45-6.53 (2Hs, m), 7.02-7.12 (1H, bs), 7.24 (1H, s), 7.27 (1H, d, J=2.5 Hz), 7.36 (1H, d, J=2.5 Hz); ¹³C NMR (300 MHz, CDCl₃): 38.55, 54.88, 54.91, 98.19, 103.65, 111.84, 115.14, 117.0, 130.23, 147.86, 155.35, 158.14, 160.40; EI-Mass: 304.8 (M⁺−1);

5-Nitro-furan-2-carboxylic acid 3,4-dimethoxy-benzylamide (23). 5-Nitro-2-furan carboxylic acid (300 mg, 1.9 mmol) and 3,4-dimethoxy-benzylamine (289 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 526 mg of product (90% yield). TLC: R_{f} 0.3 (1:1 hexane:ethyl acetate); ¹H NMR (300 MHz, CDCl₃): δ3.9 (6Hs, s) 4.19 (2Hs, d, J=6.5 Hz), 6.8-6.97 (3Hs, m), 7.31 (1H, d, J=3.4 Hz), 7.39 (1H, d, J 3.4 Hz); ¹³C NMR (300 MHz, CDCl₃): 42.91, 55.39, 55.40, 110.85, 111.02, 111.87, 115.46, 119.99, 129.11, 147.53, 148.31, 148.73, 155.59; EI-Mass: 205.0 (M⁺−1).

5-Nitro-furan-2-carboxylic acid 3,4,5-trimethoxy-benzylamide (24). 5-Nitro-2-furan carboxylic acid (300 rug, 1.9 mmol) and 3,4,5-trimethoxy-benzylamine (326 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 532 mg of product (83% yield). TLC: R_{f} 0.80 (1:1 hexane:ethyl acetate); ¹H NMR (300 MHz, CDCl₃): δ3.87 (3Hs, s), 3.89 (6Hs, s), 4.58 (2Hs, d, J=5.8 Hz), 6.59 (2Hs, s), 6.86-6.93 (1H, bs), 7.32 (1H, d, 4.2 Hz), 7.4 (1H, d, J=4.2 Hz); ¹³C NMR (300 MHz, CDCl₃): 43.42, 55.65, 60.28, 104.85, 111.88, 115.59, 132.13, 147.42, 153.01, 155.59; EI-Mass: 335.0 (M⁺−1).

5-Nitro-furan-2-carboxylic acid 1,2,3,4-tetrahydro-naphthalen-1-yl)-amide (25). 5-Nitro-2-furan carboxylic acid (300 mg, 1.9 mmol) and 1-amino(1,2,3,4-tetrahydro)naphthalene (274 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 388 mg of product (71% yield). TLC: R_{f} 0.75 (1:1 hexane:ethyl acetate); ¹H NMR (300 MHz, CDCl₃): δ1.8-2.22 (4Hs, m), 2.78-2.98 (2Hs, m), 5.33-5.45 (1H, m), 6.81-6.9 (1H, bd, J=8.3 Hz), 7.14-7.27 (3Hs, m), 7.28 (1H, d, J=3 Hz), 7.3 (1H, d, J=4.1 Hz), 7.38 (1H, d, J=4.1 Hz) ; ¹³C NMR (300 MHz, CDCl₃): 19.41, 28.53, 29.48, 47.37, 111.90, 115.55, 125.58, 125.93, 126.46, 127.24, 128.12, 128.20, 128.44, 128.90; EI-Mass: 384.9 (M⁺−1).

5-Nitro-furan-2-carboxylic acid indan-1-ylamide (26). 5-Nitro-2-furan carboxylic acid (300 mg, 1.9 mmol) and 1-amino-indane (246 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure afford 415 mg of product (80% yield). TLC: R_{f} 0.75 (1:1 hexane:ethyl acetate); ¹H NMR (300 MHz, CDCl₃): δ1.95-2.1 (1H, m), 2.62-2.76 (1H, m), 2.88-3.02 (1H, m), 3.03-3.17 (1H, m), 5.67 (1H, q, J 6.75 Hz, 13.5 Hz), 6.88-6.97 (1H, bd, J=6.75 Hz), 7.22-7.39 (m6Hs, m); ¹³C NMR (300 MHz, CDCl₃): 29.77, 33.14, 54.36, 111.9, 115.53, 123.64, 124.48, 126.45, 127.92, 141.42, 142.98, 147.51, 155.50; EI-Mass: 370.9 (M⁺−1).

5-Nitro-furan-2-carboxylic acid phenethyl-amide (27). 5-Nitro-2-furan carboxylic acid (300 mg, 1.9 mmol) and phenethylamine (239 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure afford 402 mg of product (81% yield). TLC: R_{f} 0.70 (1:1 hexane:ethyl acetate); ¹H NMR (300 MHz, CDCl₃): δ2.95 (2Hs, t, J=7.5 Hz), 3.72 (2Hs, q, J=13.8 Hz, 7.5 Hz), 6.81-6.92 (1H, bs), 7.21-7.38 (7Hs, m); ¹³C NMR (300 MHz, CDCl₃): 35.04, 40.29, 111.8, 115.26, 126.3, 128.17, 128.28, 137.58, 147.50, 155.68; EI-Mass: 258.8 (M⁺−1).

5-Nitro-furan-2-carboxylic acid [2-(4-methoxy-phenyl)-ethyl]-amide (28). 5-Nitro-2-furan carboxylic acid (300 mg, 1.9 mmol) and 4-methoxy-phenethylamine (279 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 443 mg of product (80% yield). TLC: R_{f} 0.6 (1:1 hexane:ethyl acetate); ¹H NMR (300 MHz, CDCl₃): δ2.9 (2Hs, t, J=7.1 Hz), 3.68 (2Hs, q, J=14.2 Hz, 7.1 Hz), 3.81 (3Hs, s), 6.67-6.76 (1H, bs), 6.88 (2Hs, d, J=8.6 Hz), 7.16 (2Hs, d, J=8.6 Hz), 7.25 (1H, d, J=3.8 Hz), 7.36 (1H, d, J=3.8 Hz); ¹³C NMR (300 MHz, CDCl₃): 34.12, 40.49, 54.73, 111.83, 113.69, 115.21, 129.12, 129.58, 147.58, 155.70, 157.97; EI-Mass: 288.8 (M⁺−1).

5-Nitro-furan-2-carboxylic acid(1-phenyl-ethyl)-amide (29). 5-Nitro-2-furan carboxylic acid (300 mg, 1.9 mmol) and 1-(S)-phenyl-ethylamine (245 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 422 mg of product (85% yield). TLC: R_{f} 0.75 (1:1 hexane:ethyl acetate); ¹H NMR (300 MHz, CDCl₃): δ1.65 (3Hs, d, J=7.2 Hz), 5.32 (1H, quin, J=14.0 Hz, 7.2 Hz), 6.8-6,92 (1H, bd, J=7.2 Hz), 7.24-7.45 (7Hs, m); ¹³C NMR (300 MHz, CDCl₃): 20.92, 48.69, 111.92, 115.54, 125.77, 127.31, 128.33, 141.44, 147.52, 154.84; EI-Mass: 258.8 (M⁺−1).

5-Nitro-furan-2-carboxylic acid(1-phenyl-ethyl)-amide (30). 5-Nitro-2-furan carboxylic acid (300 mg, 1.9 mmol) and 1-(R)-phenyl-ethylamine (245 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 422 mg of product (85% yield). TLC: R_{f} 0.75 (1:1 hexane:ethyl acetate); ¹H NMR (300 MHz, CDCl₃): δ1.65 (3Hs, d, J=7.2 Hz), 5.32 (1H, quin, J=14.0 Hz, 7.2 Hz), 6.8-6.92 (1H, bd, J=7.2 Hz), 7.24-7.45 (7Hs, m); ¹³C NMR (300 MHz, CDCl₃); 20.91, 48.69, 111.93, 115.54, 125.77, 127.31, 128.32, 141.47, 147.53, 154.87; EI-Mass: 258.8 (M⁺−1).

5-Nitro-furan-2-carboxylic acid [2-(3,4-dimethoxy-phenyl)-ethyl]-amide (31). 5-Nitro-2-furan carboxylic acid (300 mg, 1.9 mmol) and 2,4-dimethoxy phenethylamine (319 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 550 mg of product (90% yield). TLC: R_{f} 0.75 (1:1 hexane:ethyl acetate); ¹H NMR (300 MHz, CDCl₃): δ2.89 (2Hs, t, J=7.3 Hz), 3.69 (2Hs, q, J=14.7 Hz, 7.3 Hz), 3.86 (3Hs, s), 3.88 (3Hs, s), 6.7-6.87 (4Hs, m), 7.24 (1H, d, J=4 Hz), 7.35 (1H, d, J=4 Hz); “³C NMR (300MHz, CDCl₃): 34.58, 40.39, 55.36, 55.41, 111.06, 111.37, 111.83, 115.23, 120.15, 130.10, 147.45, 147.55, 148.71, 155.68; EI-Mass: 318.9 (M⁺−1).

5-Bromo-furan-2-carboxylic acid 4-methoxy-benzylamide (32). 5-Bromo-2-furan carboxylic acid (7a) (360 mg, 1.9 mmol) and 4-methoxy bezylamine (249 μL, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 538 mg of product (90% yield). TLC: R_{f} 0.75 (1:1 hexane:ethyl acetate); ¹H NMR (300 MHz, CD₃OD): δ3.82 (3Hs, s), 4.55 (2Hs, d, J=6.2 Hz), 6.45 (1H, d, J=3.6 Hz), 6.5-6.6 (1H, bs), 6.9 (2Hs, d, J=5.3 Hz), 7.1 (1H, d, J=3.6 Hz), 7.29 (2Hs, d, J=5.3 Hz); ¹³C NMR (300 MHz, CDCl₃):42.19, 54.76, 113.57, 113.61, 116.08, 123.81, 128.79, 129.39, 149.01, 156.54, 158.63; EI-Mass: 333.9 (M⁺+23).

5-Bromo-furan-2-carboxylic acid(3-methoxy-phenyl)-amide (33). 5-Bromo-2-furan carboxylic acid (500 mg, 2.6 mmol) and m-anisidine (292 μL, 2.6 mmol) in DMF (10 mL) was treated with EDCl (993 mg, 5.2 mmol) followed by DMAP (793 mg, 6.5 mmol). The reaction mix was stirred for 14 hr. at room temperature and worked up as explained in general procedure afforded 606 mg of product (78% yield). TLC: R_{f} 0.80 (1:1 hexane:ethyl acetate); ¹H NMR (300 MHz, CD₃OD): δ3.85 (3Hs, s), 6.51 (1H, d, J=4.0 Hz), 6.72 (1H, dd, J=8.1 Hz, 2.7 Hz), 7.12 (1H, d, J=8.1 Hz), 7.18 (1H, d, J=4.0 Hz), 7.26 (1H, t, J=8.1 Hz), 7.43 (1H, t, J=2.7 Hz), 8.0-8.1 (1H, bs); ¹³C NMR (300 MHz, CDCl₃): 54.71, 105.48, 110.07, 111.98, 113.98, 116.92, 124.42, 129.15, 137.92, 148.79, 154.61, 159.56; EI-Mass: 293.6 (M⁺−1).

5-Sulfo-furan-2-carboxylic acid(9a). A solution of AgNO₃ (1.7 g, 10 mmol) in 5 mL of water was added with stirring to a solution of NaOH (0.8 g, 20 mmol) in 5 mL of water. Sodium,5-formyl-furan-2-sulfonate (8a) (1 g, 5 mmol) was added in portions to the resulting brown mixture. The reaction mixture was stirred for 0.5 hr. at room temperature, filtered and the residue was washed with 10 mL of hot water. The chilled filtrate was neutralized with con. HCl and the product was used as such in further reactions. ¹H NMR (300 MHz, D₂O): δ7.1 (1H, d, J=4.5 Hz), 7.22 (1H, d, J=4.5 Hz); ¹³C NMR (300 MHz, CDCl₃): 112.91, 115.34, 149.42, 152.06, 164.69; EI-Mass: 190.6 (M⁺−1).

5-(3-Methoxy-phenylcarbamoyl)-furan-2-sulfonic acid (34). 5-Sulfo-furan-2-carboxylic acid (9a) (191 mg, 1 mmol) and m-anisidine (122 μL, 1 mmol) in DMF (5 mL) was treated with EDCl (382 mg, 2 mmol), DMAP (30 mg, 0.25 mmol), NEt₃ (388 μL, 3 mmol) and followed the reaction as explained above to afford 112 mg of product (38% yield). TLC: R_{f} 0.40 (20:1 chloroform:methanol); ¹H NMR (300 MHz, D₂O): δ3.61 (3Hs, s), 6.58 (1H, dd, J=8.2 Hz, 2.7 Hz), 6.81 (1H, d, J=3.5 Hz), 6.92 (1H, dd, J=8.2 Hz, 1.6 Hz), 6.98 (1H, t, J=2.7 Hz), 7.03 (1H, d, J 3.5 Hz), 7.11 (1H, t, J=8.2 Hz), 7.68 (1H, d, J=8.1 Hz); ¹³C NMR (300 MHz, CDCl₃): 54.64, 105.38, 106.29, 109.94, 111.20, 112.0, 114.69, 129.03, 138.19, 138.43, 146.70, 159.42; EI-Mass: 296.2 (M⁺−1).

5-Nitro-furan-2-carboxylic acid(3-hydroxy-phenyl)-amide (35). 5-Nitro-2-furan carboxylic acid (300 mg, 1.9 mmol) and 3-amino-phenol (208 mg, 1.9 mmol) in DMF (5 mL) was treated with EDCl (730 mg, 3.8 mmol) followed by DMAP (582 mg, 4.7 mmol). The reaction mix was stirred for 14 hr. at room temperature and worked up as explained in general procedure to afford 331 mg of product (70% yield). TLC: R_{f} 0.50 (1:1 hexane:ethyl acetate); ¹H NMR (500 MHz, CD₃OD): δ5.09 (1H, ddd, J=8.0 Hz, 2.5 Hz, 1.0 Hz), 5.59 (1H, ddd, J=8.0 Hz, 2.0 Hz, 1.0 Hz), 5.64 (1H, t, J=8.0 Hz), 5.78 (1H, t, J=2.0 Hz), 5.9 (1H, d, J=4.0 Hz), 6.45 (1H, d, J=4.0 Hz), 6.45 (1H, s); EI-Mass: 247.2 (M⁺−1).

5-Phenylsulfanyl-furan-2-carboxylic acid(3-methoxy-phenyl)-amide (36). Mixture of thiophenol (0.35 mL, 0.034 mmol) in DMF (5 mL) added NaH (0.24 g, 10.2 mmol) at 0° C., stirred for 15 min. Then slowly added 5-Bromo-furan-2-carboxylic acid(3-methoxy-phenyl)-amide 33 (1 g, 0.34 mmol) and stirring continued for 12 hr. at 150° C. Reaction mixture was treated with sat. NH₄Cl (3 mL), diluted with water (50 mL) and extracted with EtOAc (3×50 mL). The combined EtOAc fractions was washed with brine (75 mL), dried over Na₂SO₄ and concentrated in a vacuum followed by flash column purification with Pet. Ether and EtOAc in 2:1 ratio, to give 884 mg of product (80% yield). TLC: R_{f} 0.80 (1:1 hexane:ethyl acetate); ¹H NMR (500 MHz, CDCl₃): δ3.88 (3Hs, s), 6.76 (1H, dd, J=8 Hz, 2 Hz), 6.88 (1H, d, J=3.5 Hz), 7.16 (1H, dd, J=8 Hz, 1.5 Hz), 7.28-7.35 (4Hs, m), 7.36-7.41 (2Hs, m), 7.47 (1H, t, J=2.5 Hz), 8.04-8.11 (1H, bs); ¹³C NMR (300 MHz, CDCl₃): 54.76, 105.35, 110.23, 111.89, 116.39, 120.37, 126.78, 128.1, 128.91, 129.19, 135.01, 137.84, 146.38, 149.89, 154.99, 159.62; EI-Mass: 348.3 (M⁺+23).

5-Nitro-furan-2-carboxylic acid(3-benzyloxy-phenyl)-amide (37). Compound 35 (150 mg, 0.6 mmol) was dissolved in dry THF (5 mL) and K₂CO₃ (167 mg, 1.2 mmol) followed by benzyl bromide (146 μL, 1.2 mmol). The reaction mixture was stirred for 12 hr. at room temperature. The reaction mixture was diluted with 30 mL ethyl acetate and washed with H₂O (25 mL), brine (25 mL). The ethyl acetate was dried and concentrated. The crude product was purified with flash column using 15% EtOAc in hexane to afford 147 mg of product (72% yield). TLC: R_{f} 0.82 (1:1 hexane:ethyl acetate); ¹H NMR (500 MHz, CDCl₃): δ5.16 (2Hs, s), 6.9 (1H, dd, J=8.3, 2.4, 1.0 Hz), 7.23 (1H, dd, J=8.1, 2.0, 0.8 Hz), 7.35 (1H, t, J=8 Hz), 7.39 (1H, dt, J=7.0, 2.5 Hz), 7.42-7.44 (4Hs, m), 7.5-7.52 (2Hs, m), 7.56 (1H, t, J=2.2 Hz), 8.26-8.3 (1H, bs); ¹³CNMR (300 MHz, CDCl₃): 69.59, 106.51, 111.82, 112.1, 112.26, 116.24, 126.94, 127.5, 128.06, 129.48, 136.15, 136.99, 147.28, 153.37, 158.93; EI-Mass: 337.6 (M⁺−1).

5-Benzenesulfinyl-furan-2-carboxylic acid(3-methoxy-phenyl)-amide (38) and 5-Benzenesulfonyl-furan-2-carboxylic acid(3-methoxy-phenyl)-amide (39). A mixture of compound 36 (0.1 g, 0.3 mmol) and NaHCO₃ (0.116 g, 1.3 mmol) in CH₂Cl₂ (5 mL) at 0° C. treated with m-chloroperbenzoicacid (0.116 g, 0.67 mmol) and stirred for 3 hr. The reaction mixture was quenched with dil. Aq. NH₄OH solu. (5 mL) and diluted with CH₂Cl₂ (30 mL). The organic layer was quenched with dil. Aq. NH₄OH solu. (30 mL), water (30 mL), brine (30 mL) and dried over Na₂SO₄. The organic solution was concentrated in vacuum followed by flash column purification with Pet. Ether and EtOAc in 5:1 ratio afforded the products 31 mg of 38 and 38 mg of 39 in 30% and 35% yields respectively. 5-Benzenesulfinyl-furan-2-carboxylic acid (3-methoxy-phenyl)-amide (38): TLC: R_{f} 0.30 (1:1 hexane:ethyl acetate); ¹H NMR (500 MHz, CDCl₃): δ4.87 (3H, s), 6.78 (1H, dd, J=8 Hz, 2.5 Hz), 6.81 (1H, d, J=3.5 Hz), 7.15 (1H, dd, J=8 Hz, 1.5 Hz), 7,29 (1H, d, J=3.5 Hz), 7.32 (1H, d, J=8 Hz), 7.42 (1H, t, J=2 Hz), 7.64 (3Hs, t, J=3 Hz), 7.8 (2Hs, dd, J=6 Hz, 3.5 Hz, 2.5 Hz), 8.2 (1H, s); ¹³C NMR (300 MHz, CDCl₃): 54.81, 105.30, 110.49, 111.77, 115.16, 116.54, 124.47, 129.07, 129.29, 131.50, 137.45, 140.35, 150.73, 154.32, 159.68, EI-Mass: 340.6 (M⁺−1); 5-Benzenesulfonyl-furan-2-carboxylic acid(3-methoxy-phenyl)-amide (39): TLC: R_{f} 0.70 (1:1 hexane:ethyl acetate); ¹H NMR (500 MHz, CDCl₃): δ4.85 (3H, s), 6.79 (1H, dd, J=8 Hz, 2.2 Hz, 1.7 Hz), 7.18 (1H, dd, J=7.4 Hz, 1.5 Hz), 7.28-7.34 (3Hs, m), 7.4 (1H, t, J=2.5 Hz), 7.62 (2Hs, t, J=8 Hz), 7.72 (1H, t, J=7 Hz), 8.09 (2Hs, d, J=7 Hz), 8.22 (1H, s); ¹³C NMR (300 MHz, CDCl₃): 54.81, 105.57, 110.66, 112.02, 115.29, 118.42, 127.47, 129.09, 129.3, 133.85, 137.22, 138.65, 150.34, 150.63, 154.07, 159.67; EI-Mass: 356.5 (M⁺−1).

5-(3-Methoxy-phenylcarbamoyl)-furan-2-carboxylic acid ethyl ester (40). A flame evacuated three neck round bottom flask fitted with reflux condenser, containing magnesium (32 mg, 1.3 mmol) under argon atmosphere was added dry THF (3 mL), followed by 5-Bromo-furan-2-carboxylic acid(3-methoxy-phenyl)-amide 33 (0.2 g, 0.67 mmol) in 2 mL THF and ethyl bromide (72 mg, 0.67 mmol). The resulting mixture was stirred for 15 min. at 50° C. and cooled to 0° C., then added ethyl chloroformate (0.145 g, 1.3 mmol), continued stirring at room temperature for 5 hr. Then aq. Sat.NH₄Cl solu. (1 mL) was added to the reaction mixture and diluted with EtOAc (50 mL). The EtOAc was washed with brine (50 mL) dried over Na₂SO₄, concentrated under vacuum and purified by flash-column to give product only 39 mg (20% yield). TLC: R_{f} 0.75 (1:1 hexane:ethyl acetate); ¹H NMR (300 MHz, CDCl₃): δ1.21 (3Hs, t, J=8.6 Hz), 3.56 (3Hs, s), 4.25 (2Hs, q, J=8.6 Hz, 14.14 Hz), 6.51 (1H, dd, J=1.69 Hz, 3.38 Hz), 6.81 (1H, t, J=1.69 Hz), 6.87 (1H, ddd, J=0.65 Hz, 2.6 Hz, 7.54 Hz), 6.92 (1H, ddd, J=0.65 Hz, 2.6 Hz, 7.54 Hz), 7.03 (1H, dd, J=0.65 Hz, 3.38 Hz), 7.33 (1H, t, J=7.54 Hz), 7.55 (1K, dd, J=0.65 Hz, 1.69 Hz); ¹³C NMR (300 MHz, CDCl₃): 13.47, 54.87, 62.80, 111.69, 113.15, 113.5, 118.29, 119.66, 129.27, 138.74, 145.12, 159.68, 160.01; EI-Mass: 312.1 (M⁺+23).

Preparation of 5-Nitro-furan-2-carbonyl chloride (6a). 5-Nitro-furan-2-carboxylic acid(942 mg, 6 mmol) in DCM (10 mL) was treated with oxalylchloride (1.046 mL, 12 mmol) followed by 2 drops of DMF and stirred at room temperature for 4 hr. The reaction mix was concentrated in vacuum to obtain acid chloride.

General procedure for preparation of amides (6a).

Method 1: 5-Nitro-furan-2-carbonyl chloride (526 mg, 3 mmol) in DMF (5 mL) was added to amine (3 mmol) in pyridine (5 mL) and reaction was carried out at 60° C. The reaction was diluted with EtOAc (100 ml), washed with 10% aqueous NaHCO₃ (2×50 ml), water (2×50 ml) and brine (2×50 ml). The organic phase was dried over Na₂SO₄, filtered and concentrated, followed by flash column purification, which provided corresponding amides.

Method 2: 5-Nitro-furan-2-carbonyl chloride (930 mg, 5.3 mmol) in CH₂Cl₂ (10 ml) was added amine (5.3 mmol, 1 equiv.) in Et₃N (3 ml) and the mixture was stirred for 14 hrs. at 47° C. Reaction was followed by TLC, after completion of reaction 100 ml of EtOAc was added and washed with 10% aqueous NaHCO₃ (2×50 ml), water (2×50 ml) and brine (2×50 ml). The organic phase was dried over Na₂SO₄, filtered and concentrated followed by flash column purification, which provided corresponding amides.

5-Nitro-furan-2-carboxylic acid pyrazin-2-ylamide (48). 5-Nitro-furan-2-carbonyl chloride (526 mg, 3 mmol) in DMF (5 ml) was added aminopyrazine (285 mg, 3 mmol) followed by pyridine (5 ml). The reaction was stirred for 14 hr. at 60° C. The reaction was followed as explained in method 1 above to yield 120 mg (17%) of compound 48; TLC: R_{f} 0.30 (1:1 hexane:ethylacetate). ¹HNMR (500 MHz, CDCl₃): δ7.46 (1H, d, J=3.91 Hz), 7.49 (1H, d, J=3.91 Hz), 8.37-8.40 (1H, m), 8.49 (1H, d, J=2.44 Hz), 8.76-8.86 (1H, bs), 9.65 (1H, d, J=1.47 Hz); ¹³CNMR (300 MHz, mixture of CD₃OD:CDCl₃ 1:3): 111.90, 117.33, 136.68, 140.00, 142.26; EI-Mass: 234.9 (M+1 232.9 (M⁺−1); IR: 1701, 3110, 3180 cm⁻¹.

5-nitro-furan-2-carboxylic acid(pyridin-2-ylmethyl)-amide (49). To the mixture of 5-Nitro-furan-2-carbonyl chloride (930 mg, 5.3 mmol) in CH₂Cl₂ (10 ml) was added 2-aminomethylpyridine (0.54 ml, 5.3 mmol) in Et₃N (3 ml) and stirred for 14 hrs. at 47° C. The reaction was followed as explained in method 2 to yield 1.12 gm (85%) of product 49. TLC: R_{f} 0.11 (1:1 hexane:ethyl acetate); ¹HNMR (500 MHz, CDCl₃): δ4.83 (2H, d, J=5.37 Hz), 7.32 (1H, d, J=3.91 Hz), 7.38 (1H, d, J=3.66 Hz), 7.39-7.42 (1H, m), 7.48 (1H, d, J=7.81 Hz); 7.86 (1H, dt, J=1.71 Hz), 8.10-8.22 (1H, bs), 8.65 (1H, d, J=4.8 Hz); ¹³CNMR (300 MHz, CDCl₃): 43.75, 111.75, 121.55, 122.16, 136.40, 147.60, 148.70, 154.70, 155.79; EI-Mass: 248 (M⁺+23); IR: 1670, 3305 cm⁻¹.

5-nitro-furan-2-carboxylic acid(4-methoxy-benzothiazol-2-yl)-amide (50). To the mixture of 5-Nitro-furan-2-carbonyl chloride (667 mg, 3.8 mmol) in CH₂Cl₂ (5 ml) was added 2-amino 4-methoxy benzothiazole (684 mg, 3.8 mmol) followed by pyridine (5 ml) and the reaction mixture was stirred for 14 hr. at room temperature. The reaction was followed as explained in method 2 to yield 480 mg (29%) of compound 50. TLC: R_{f} 0.37 (1:1 hexane:ethyl acetate). ¹HNMR (500 MHz, CD₃OD): δ4.03 (3H, s), 7.04 (1H, d, J=8.06 Hz), 7.30 (1H, t, J=8.06 Hz), 7.44 (1H, d, J=8.06 Hz), 7.53 (1H, m), 7.59 (1H, d, J=3.66 Hz); ¹³CNMR (300 MHz, CDCl₃): 29.15, 55.36, 106.56, 111.59, 113.07, 117.70, 125.12; EI-Mass: 318 (M⁺−1); IR: 1561,1701 cm⁻¹.

5-nitro-furan-2-carboxylic acid(6-methoxy-pyrimin-4-yl)-amide (51). To the mixture of 5-nitro-furan-2-carbonyl chloride (667 mg, 3.8 mmol) in CH₂Cl₂ (5 ml) was added 4-amino 6-methoxy pyrimidine (475 mg, 3.8 mmol) followed by pyridine (5 ml) and reaction was stirred for 14 hr. at 50° C. The reaction was followed as explained in method 2 to yield 550 mg (40%) of compound 51. TLC: R_{f} 0.53 (1:1 hexane:ethyl acetate). ¹HNMR (500 MHz, CDCl₃): δ4.04 (3H, s), 7.44 (2H, q, J=4.04, 8.06 Hz), 7.65 (1H, d, J=0.97 Hz) 8.58 (1H, d, J=0.98 Hz), 8.85 (1H, s); ¹³CNMR (300 MHz, CDCl₃): 53.77, 95.60, 111.79, 117.34, 145.98, 154.13, 156.21, 170.80; EI-Mass: 263 (M⁺−1); IR: 1576, 1684, 3123 cm⁻¹.

5-nitro-furan-2-carboxylic acid 2-methoxy-benzylamide (52). To the mixture of 5-nitro-furan-2-carbonyl chloride (877 mg, 5 mmol) in CH₂Cl₂ (10 ml) was added to 2-methoxy benzyl amine (0.646 ml, 5 mmol) in Et₃N (3 ml) and reaction was stirred for 14 hr. at room temperature. The reaction was followed as explained in method 2 to yield 685 mg (49%) of compound 52. R_{f} 0.53 (1:1 hexane:ethyl acetate). ¹HNMR (500 MHz, CDCl₃): δ3.94 (3H, s), 4.65 (2H, d, J=5.86 Hz), 6.96 (2H, dd, J=7.09, 7.32 Hz), 10-7.20 (1H, bs), 7.26 (1H, d, J=3.66 Hz), 7.31-7.36 (3H, m); ¹³CNMR (300 Mhz, CDCl₃): 38.94, 54.88, 109.91, 109.98, 111.87, 115.19, 120.18, 124.52, 128.79, 129.33, 147.81, 155.46, 157.07; EI-Mass: 299.3 (M⁺+23); IR: 1676, 3307 cm⁻¹.

5-nitro-furan-2-carboxylic acid 2,3-dimethoxy-benzylamide (53). To the mixture of 5-nitro-furan-2-carbonyl chloride (877 mg, 5 mmol) in CH₂Cl₂(10 ml) was added to 2,3-dimethoxy benzyl amine (0.734 ml, 5 mmol) in Et₃N (3 ml) and reaction was stirred for 14 hr. at room temperature. The reaction was followed as explained in method 2 to yield 830 mg (54%) of compound 53. R_{f} 0.48 (1:1 hexane:ethyl acetate). ¹HNMR (500 MHz, CDCl₃): δ3.90 (3H, s), 3.95 (3H, s) 4.65 (2H, d, J=6.10 Hz), 6.92 (2H, dd, J=1.46, 7.05 Hz), 6.95 (1H, dd, J=1.46, 7.81 Hz), 7.03-7.09 (3H, m); ¹³CNMR (300 MHz, CDCl₃): 38.91, 55.21, 60.19, 111.88, 115.27, 120.84, 123.63, 130.04, 146.76, 147.66, 152.11, 155.54; EI-Mass: 329 (M⁺+23), 305 (M⁺−1); IR: 1671, 3323 cm⁻¹.

### Biological Results

To develop the structure activity relationship of 1, 43 compounds were synthesized and tested for enzymatic inhibition of Glf and for MIC activity against M tuberculosis (Table 1). Although most of the tested compounds inhibited Glf, the best inhibitors of Glf were 10 and 13, both 3-substituted anilinyl nitrofuran amides. In general substituted nitrofuranyl benzylamides, were less active than the analogous anilinyl amides. The nitrofuranyl group was shown to be required for Glf activity.

The MIC of the nitrofuranyl amides against M. tuberculosis H37Ra was determined by the micro broth dilution method using microtiter plates. M. tuberculosis was grown in Middlebrook 7H9 medium to an OD⁶⁵⁰ of 0.4-0.6 and a dilution made to an OD₆₅₀ of 0.01. 100 μl of these cells are then added to microtiter well containing serial dilutions of the nitrofuranyl amides. The cell are then incubated at 37° C. for 7 days and visually examined for growth. MIC₉₀was determined for wells with greater than 90% inhibition of growth.

Cytotoxicity was determined using alamar blue assay against Vero cells.

An assay for Maximum Tolerated Dose (MTD) was also utilized. Three healthy mice were given orally one single dose of the compound and are observed at regular times for any adverse effects. Three different concentrations are tested, generally at 100, 300 and 500 mg/kg. The latter dose is about twice to five times the dose used for efficacy testing of the compound in mice. After 7 days of observation the mice are sacrificed and the organs are studied for any adverse effects. In case of abnormalities the organs are fixed in formalin and given for extensive pathology analysis.

Mice used as a GKO mouse model were infected via low dose aerosol to reproducibly deliver M. tuberculosis in the alveolar regions of the lungs in low numbers to mimic the realistic disease in humans. Treatment was initiated 18 days postinfection for 9 daily treatments for one single dose (at 300 mg/kg). Bacterial load was determined 28 days postinfection in lungs and spleens of the mice. To determine whether the results have statistical significant value, statistics on the data for every compound were performed using the SigmaStat™ program. Due to the short term treatment regimen, fluctuations in CFU within mice from one treatment group are limited and a reduction of ˜0.3 Log10 CFU in the lungs is considered statistically significant.

The MIC activity of the series showed a strong structure activity relationship with the nitro group being required for activity in all cases. Anilinyl, benzyl and phenethyl amides all had significant activity, with increased activity compared to saturated cyclohexylamide 15 and adamantylamide amide 16. Heteroaromatic substitutions such as pyridines 44, 45, 46, pyrazole 47, pyrazine 48, furfuryl amide 18 all were less active than the corresponding aniline amide 17. Tertiary amides 41, 42 were less active than their corresponding secondary amides 14, 17. The most active series was the methoxy substituted benzylamides with a range of relative activities 4-methoxybenzyl 20>3,4,-dimethoxy benzyl 23>2,4-dimethoxy benzyl 22>3,4,5-trimethoxy benzyl 24>2,3 dimethoxy benzyl 53>2-methoxy benzyl 52. The activity of this series shows a clear preference for 4-methoxy substituted systems. Compounds in the methoxy benzyl series showed the highest therapeutic index principally due to their low MIC values. The nitrofuranyl amides when tested against other bacteria Mycobacterium smegmatis, Staphylococcus aureus or Escherichia coil in MIC assays were all inactive.

Added to Table 1 are the predictive pharmacokinetic values of CLogP, Solubility and Protein binding. These values were used to aid the decision as to which compounds were to advance towards in vivo testing. Volsurf was used to predict the solubility and protein binding. The predicted values for protein binding are in percent protein bound. An ideal antimicrobial agent would have a low percent protein bound prediction, as protein binding can influence such factors as delivery to target tissue, effective MIC concentration in human serum, drug interactions, metabolism, and clearance. The predicted protein binding values for this series is acceptable.

After examining MIC, therapeutic index, Glf inhibition, CLogP, calculated solubility and protein binding data for the compound series, compounds 10, 12, 13, 20, 23 were selected for in vivo testing.

Maximum tolerated dosing was performed on these selected compounds. An escalating dose of drug (100, 300 and 500 mg/kg) was given to mice by oral gavage. Compounds 10, 12 and 23 showed no effect at the maximum dose. 13 showed some pathology at 500 mg/kg.

Subsequently, the four compounds were tested for efficacy against M. tuberculosis at a dose (see Table 1) lower than the MTD in infected C57BL/6 Interferon-γ gene depleted mice (see below). The results of the experiment are presented in Table 2 below.

The sub-microgram MIC activity of some of the nitrofuranyl amides has lead to the exploration of their usage as anti-tuberculosis agents. The MIC activity of this series compares well with front-line anti-tuberculosis agents such as isoniazid (MIC₉₀ 0.05 μg/mL) and ethambutol (MIC₉₀ 0.78μg/mL) and they have an acceptable therapeutic index (Table 1). Four compounds passed the maximum tolerated dose assay and compound 23 showed significant oral activity against M. tuberculosis in the mouse infection model.

Like PA824, their activity is restricted to mycobacteria of the mycobacterium tuberculosis complex with no appreciable activity against M. smegmatis, S. aureus or E. coil, a property that believed to be desirable for the development of new tuberculosis treatments.

| | | | TB MIC | | Therap. | | | Protein |
|---|---|---|---|---|---|---|---|---|
| Compound | R₁ | HNR₂R₃ | (μg/mL) | ToxIC₅₀ | Index² | CLogP^{b} | Solub.^{c} | Binding^{d} |
| 1 | NO₂ | 3-chloro-4-methoxy aniline | 1.6 | | | 2.554 | −3.50 | 61.91 |
| 10 | NO₂ | 3-chloro-aniline | 038 | 6.86 | 8.6 | 2.804 | −3.52 | 68.45 |
| 11 | NO₂ | 3-bromo-aniline | 1.6 | 15.37 | 9.6 | 2.954 | −3.59 | 68.91 |
| 12 | NO₂ | 3-fluoro-aniline | 0.8 | 9.29 | 11.6 | 2.234 | −3.30 | 66.61 |
| 13 | NO₂ | 3-anisidine | 0.8 | 18.63 | 23.3 | 1.975 | −3.39 | 63.60 |
| 14 | NO₂ | 4-anisidine | 0.4 | 21.44 | 53.6 | 1.975 | −3.44 | 63.09 |
| 15 | NO₂ | cyclohexylamine | 3.1 | 9.08 | 2.9 | 2.243 | −3.00 | 64.78 |
| 16 | NO₂ | adamantyl amine | 3.1 | 9.43 | 3.0 | 2.871 | −3.57 | 74.18 |
| 17 | NO₂ | aniline | 0.8 | 2.19 | 2.7 | 2.001 | −3.18 | 67.46 |
| 18 | NO₂ | furfuryl amine | 6.25 | 4.08 | 0.7 | 1.356 | −2.92 | 62.01 |
| 19 | NO₂ | 4-amino-benzonitrile | 0.8 | 3.53 | 4.4 | 1.342 | −4.46 | 75.07 |
| 20 | NO₂ | 4-methoxy-benzylamine | 0.1 | 16.36 | 163.6 | 2.099 | −3.72 | 65.28 |
| 21 | NO₂ | 2-chloro-benzylamine | 1.6 | 11.75 | 7.3 | 2.893 | −3.36 | 68.69 |
| 22 | NO₂ | 2,4-dimethoxy-benzylamine | 0.4 | 5.38 | 13.5 | 2.188 | −3.65 | 59.13 |
| 23 | NO₂ | 3,4-dimethoxy-benzylamine | 0.2 | 18.17 | 90.9 | 1.838 | −3.77 | 63.68 |
| 24 | NO₂ | 3,4,5-trimethoxy-benzyfamine | 0.8 | 23.59 | 29.5 | 1.480 | −4.12 | 65.57 |
| 25 | NO₂ | 1-amino-1,2,3,4-tetrahydronaphthalene | 3.1 | 9.03 | 2.9 | 3.153 | −3.77 | 77.18 |
| 26 | NO₂ | 1-amino-indane | 3.1 | 10.28 | 3.3 | 2.594 | −3.65 | 77.02 |
| 27 | NO₂ | phenethylamine | 1.6 | 25.23 | 15.7 | 2.309 | −3.76 | 75.35 |
| 28 | NO₂ | 4-methoxy-phenethylamine | 0.8 | 20.45 | 25.6 | 2.228 | −4.19 | 71.20 |
| 29 | NO₂ | (S)-1-phenyf-ethylamine | 1.6 | 25.63 | 16.0 | 2.489 | −3.64 | 74.10 |
| 30 | NO₂ | (R)-1-phenyl-ethylamine | 3.1 | 23.59 | 7.6 | 2.489 | −3.44 | 71.13 |
| 31 | NO₂ | 3,4-dimethoxy-phenethylamine | 0.4 | 27.44 | 68.6 | 1.967 | −4.31 | 69.68 |
| 32 | Br | 4-methoxy-benzylamine | 200 | 90.54 | 0.5 | 2.989 | −3.85 | 69.41 |
| 33 | Br | 3-anisidine | 100 | 61.19 | 0.6 | 2.864 | −3.24 | 65.25 |
| 34 | SO₃H | 3-anisidine | 200 | | | −0.886 | −2.51 | 18.14 |
| 35 | NO₂ | 3-amino-phenol | 1.6 | 12.5 | 7.8 | 1.334 | −3.20 | 60.94 |
| 36 | SPh | 3-anisidine | 25 | 25.69 | 1.0 | 4.303 | −4.58 | 84.77 |
| 37 | NO₂ | 3-benzyloxy-aniline | 12.5 | | | 3.743 | −5.15 | 87.00 |
| 38 | SOPh | 3-anisidine | 6.25 | 65 | 10.4 | 2.376 | −4.25 | 61.04 |
| 39 | SO₂Ph | 3-anisidine | 6.25 | 25.85 | 4.1 | 2.736 | −4.11 | 51.50 |
| 40 | CO₂Et | 3-anisidine | 400 | 200 | 0.5 | 2.665 | −3.87 | 64.29 |
| 41 | NO₂ | N-methyl-aniline | 3.12 | | | 1.457 | −2.94 | 66.83 |
| 42 | NO₂ | N-methyl-4-anisidine | 6.25 | | | 1.406 | −3.15 | 60.45 |
| 43 | NO₂ | 2,3-dihydro-indole | 0.8 | | | 3.249 | −3.52 | 75.02 |
| 44 | NO₂ | 2-amino pyridine | 3.12 | | | 1.051 | −3.12 | 56.21 |
| 45 | NO₂ | 3-amino pyridine | 6.25 | | | 1.051 | −2.83 | 55.33 |
| 46 | NO₂ | 4-amino pyridine | 3.12 | | | 1.051 | −2.92 | 55.36 |
| 47 | NO₂ | 3-amino pyrazole | 6.25 | | | 0.601 | −2.97 | 48.70 |
| 48 | NO₂ | 2-amino pyrazine | 6.25 | | | 0.286 | −2.90 | 47.18 |
| 49 | NO₂ | 2-amino methyl pyridine | 0.8 | | | 0.683 | −3.02 | 57.31 |
| 50 | NO₂ | 2-amino-4-methoxy-benzothiazole | 1.6 | | | 2.801 | −3.96 | 74.30 |
| 51 | NO₂ | 4-amino-6-methoxy-pyrimidlne | 1.6 | | | 1.226 | −3.33 | 53.13 |
| 52 | NO₂ | 2-methoxy-benzylamine | 1.6 | | | 2.099 | −3.30 | 66.45 |
| 53 | NO₂ | 2,3-dimethoxy-benzylamine | 1.2 | | | 1.838 | 3.46 | 64.36 |

TABLE 2
Determination of viable M. tuberculosis in spleens and lungs
of infected mice after a 8-day drug treatment regimen

| Compound | | Lungs ± SEM | Spleen ± SEM |
|---|---|---|---|
| Untreated controls | | 8.0 ± 0.17 | 6.7 ± 0.16 |
| Isoniazid | | 5.5 ± 0.21 | 3.2 ± 0.21 |
| (25 | mg/kg) | | |
| Metronidazole | | 8.5 ± 0.18 | 6.9 ± 0.04 |
| (150 | mg/kg) | | |
| 10 | | 8.3 ± 0.21 | 7.3 ± 0.12 |
| (300 | mg/kg) | | |
| 12 | | 8.4 ± 0.24 | 7.0 ± 0.11 |
| (300 | mg/kg) | | |
| 13 | | 8.0 ± 0.3 | 7.2 ± 0.07 |
| (150 | mg/kg) | | |
| 23 | | 6.5 ± 0.25 | 6.0 ± 0.19 |
| (300 | mg/kg) | | |

(SEM = standard error)

### Example 2

Example 1 describes a novel set of nitrofuranyl amides with potent antituberculosis activity. Compounds in this series were easy to synthesize, had a good therapeutic index, were active against aeorobically and anaerobically grown bacilli and were not cross resistant with other clinically used anti mycobacterial drugs.

The compound 5-Nitro-furan-2-carboxylic acid 3,4-dimethoxy-benzylamide (23) shown in Table 3 below specifically had significant oral activity in a mouse model of tuberculosis infection, as demonstrated in Example 1. Very few compounds have been described in the art with this level of in vitro activity against tuberculosis.

Without wishing to be limited by theory, it is hypothesized, based on analysis of these structures and their physical properties that oral bioavailability could potentially be limited in some cases for these compounds due to poor solubility and high crystal energy of the nitrofuranyl amide series. Such issues have been encountered in the development of other synthetic antimicrobial agents, most notably in the development of the fluoroquinolone class of antibiotics.

Therefore, Example 2 describes the synthesis and evaluation of related derivative compounds. These novel compounds are cyclic secondary amine substituted phenyl and benzyl nitrofuranyl amides (see Table 3) and are shown below to be effective novel anti tuberculosis agents.

The synthesis of these compounds is divided in to two classes: phenyl amides and benzyl amides to which were added a variety of cyclic secondary amines.

TABLE 3
Structures of Lead Compound and Amide Derivatives

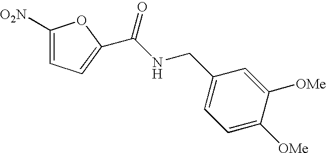

5-Nitro-furan-2-carboxylic acid
3,4-dimethoxy-benzylamide (23)

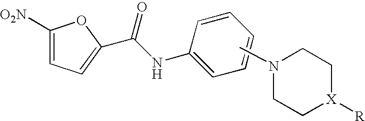

phenyl amides

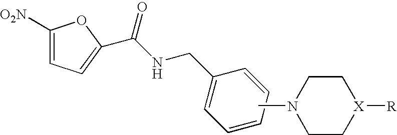

benzyl amides

### Methods and Materials

All the anhydrous solvents and starting materials were purchased from Aldrich Chemical Company (Milwaukee, Wis., U.S.A.). All reagent grade solvents used for chromatography were purchased from Fisher Scientific (Suwanee, Ga., U.S.A.) and FLASH™ column chromatography silica cartridges were obtained from Biotage Inc. (Lake Forest, Va., U.S.A.). The reactions were monitored by thin layer chromatography (TLC) on pre-coated Merck 60 F₂₅₄ silica gel plates and visualized using UV light (254 nm). Biotage FLASH 25+™ column chromatography system was used to purify mixtures. All ¹H and ¹³C NMR spectra were recorded on a Bruker ARX-300 (300 and 75 MHz for ¹H and ¹³C NMR, respectively; Billerica, Mass., U.S.A.) or Varian INOVA-500™ (500 and 125 MHz for ¹H and ¹³C NMR₁ respectively; Palo Alto, Calif., U.S.A.) spectrometer. Chemical shifts are reported in ppm (δ) relative to residual solvent peak or internal standard (tetramethylsilane) and coupling constants (J) are reported in hertz (Hz). Mass spectra were recorded on a Bruker ESQUIRE LCMS™ using ESI. Purity of the final products was confirmed before testing by analytical HPLC using a Alltech (Deerfield, Ill., U.S.A.) platinum C-18 reverse phase column (4.5×150mm) and a H₂O (0.1% TFA) to acetonitrile 0-100% linear gradient at a flow rate of 1.0 mL min⁻¹ and UV detection at 254 nm.

### Scheme 1

Referring now to Scheme 1, synthesis of the substituted phenyl amides involved a three reaction sequence of nucleophilic aromatic substitution, nitroreduction and acylation with the nitrofuranoic acid chloride. Accordingly, the fluorine of 3 or 4-fluoro nitrobenzene was substituted with secondary amides morpholine, 1-methyl-piperazine, 1-benzyl piperazine, 4-benzyl piperadine and 1-(2-pyridyl)piperazine to give corresponding substituted nitrobenzenes 22b-31b with yields 78%-95%. The substitution on p-fluoro nitrobenzene was faster than meta-substitution 8 hours compared with 24 hours, respectively. The nitro functional group of compounds 22b -31b, except compounds 24b and 29b were reduced by catalytic hydrogenation to give anilines 32b -39b in quantitative yields. The amines 24b and 29b were reduced using SnCl₂.2H₂O to their corresponding amides 40b -41b (both in 82% yields) due to sensitivity of the benzyl substituted piperazines to hydrogenation. Finally, all the amines 32b -39b were treated with 5-nitro-furan-2-carbonyl chloride to give desired phenyl amides 54-63 in 82-90% yields.

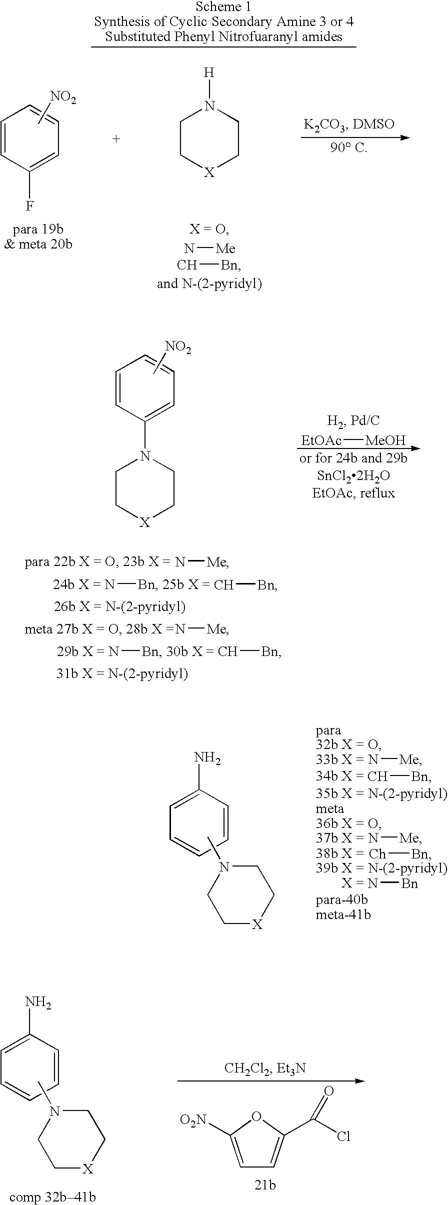

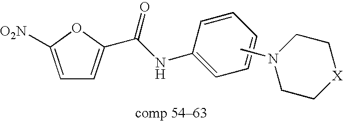

### Scheme 2

Referring now to Scheme 2, the benzyl amide series was prepared by employing the similar pattern of reactions of nucleophilic aromatic substitution, reduction followed by acylation. In this case a cyano group was used as electron withdrawing group to facilitate the substitution. Accordingly, the fluorine of the 3 or 4-fluoro benzonitrile was substituted with corresponding cyclic secondary amines in DMSO at 90° C. and in. the presence of potassium carbonate to give compounds 42b -48b in. a 83-96% yield. The substituted benzonitriles are subjected to reduction using Red-Al reagent to afford corresponding crude amines, which are further treated without purification with 5-nitro-furan-2-carbonyl chloride to give benzyl amides 64-70 in. 69-86% yields.

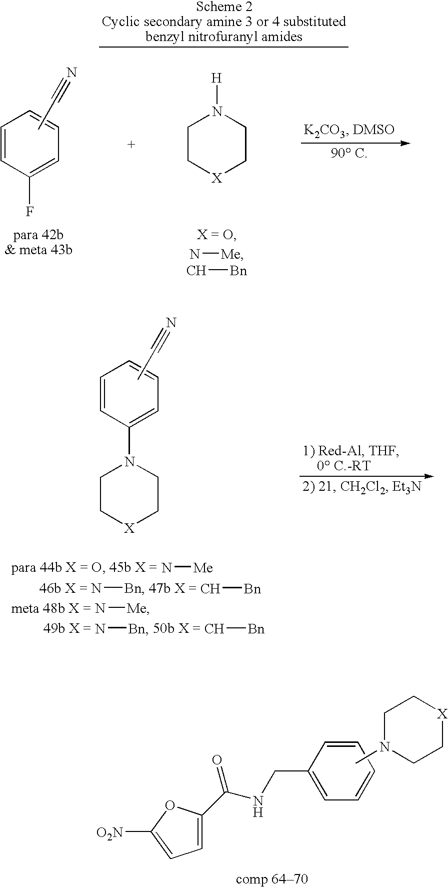

General procedure for preparation of 22b -31b and 44b -50b. To a mixture of substituted fluoro benzene (1 eq.) and K₂CO₃ (1.5 eq.) in dimethyl sulfoxide (7 mL/g) was added secondary amine (2 eq.). The reaction mixture was stirred at 90° C. and followed by TLC. After completion of reaction, the mixture was diluted with ethyl acetate (60 mL/g), and washed with water (2×50 mL/g), followed by brain (50 mL/g). The ethyl acetate fraction was dried over Na₂SO₄ and concentrated. The crude products were purified by flash column chromatography to afford pure products.

4-(4-Nitro-phenyl)-morpholine (22b). To a mixture of 4-fluoro nitro benzene 19b (425mg, 3.01 mmol) and K₂CO₃ (623 mg, 4.52 mmol) in dimethyl sulfoxide (3 mL) was added morpholine (0.52 mL, 6.02 mmol) and the reaction continued as described above to afford amine 595 mg of 22b in 95% yields. ¹H-NMR (500 MHz, CDCl₃): δ 3.37 (4Hs, t, J=4.88 Hz), 3.86 (4Hs, t, J=5.12 Hz), 6.83 (2Hs, d, J=9.52 Hz), 8.14 (2Hs, d, J=9.52 Hz); ESI-MASS: 231.0 (M+23).

1-Methyl-4-(4-nitro-phenyl)-piperazine (23b). To a mixture of 4-fluoro nitro benzene 19b (425 mg, 3.01 mmol) and K₂CO₃ (623 mg, 4.52 mmol) in dimethyl sulfoxide (3 mL) was added 1-methyl piperazine (0.66 mL, 6.02 mmol) and the reaction continued as described above to afford 612 mg of amine 23b in 92% yields. ¹H-NMR (500 MHz, CDCl₃): δ 2.36 (3Hs, s), 2.55 (4Hs, t, J=5.12 Hz), 3.44 (4Hs, t, J=5.37 Hz), 6.83 (2Hs, d, J=9.52 Hz), 8.12 (2Hs, d, J=9.52 Hz); ESI-MASS: 222.1 (M+1).

1-Benzyl-4-(4-nitro-phenyl)-piperazine (24b). To a mixture of 4-fluoro nitro benzene 19b (425 mg, 3.01 mmol) and K₂CO₃ (623 mg, 4.52 mmol) in dimethyl sulfoxide (3 mL) was added 1-benzyl piperazine (1.04 mL, 6.02 mmol) and the reaction continued as described above to afford amine 805 mg of 24b in 90% yields. ¹H-NMR (500 MHz, CDCl₃): δ 2.59 (4Hs, t, J=4.88 Hz), 3.42 (4Hs, t, J=5.12 Hz), 3.57 (2Hs, s), 6.81 (2Hs, d, J=7.32 Hz), 7.29 (1H, sextet, J=1.22 Hz), 7.34 (4Hs, d, J=7.39 Hz), 8.12 (2Hs, d; J=7.32 Hz); ESI-MASS: 298.2 (M+1).

4-Benzyl-1-(4-nitro-phenyl)-piperidine (25b). To a mixture of 4-fluoro nitro benzene 19b (425 mg, 3.01 mmol) and K₂CO₃ (623 mg, 4.52 mmol) in dimethyl sulfoxide (3 mL) was added 4-benzyl piperidine (1.06 niL, 6.02 mmol) and the reaction continued as described above to afford amine 847 mg of 25b in 95% yields. ¹H-NMR (500 MHz, CDCl₃): δ 1.33 (2Hs, dq, J=3.9, 12.45, 23.92 Hz), 1.74-1.88 (3Hs, m), 2.57 (2Hs, d, J=6.83 Hz), 2.91 (2Hs, t, J=15.13 Hz), 3.93 (2Hs, d, J=13.18 Hz), 6.78 (2Hs, d, J=9.52 Hz), 7.15 (2Hs, d, J=7.08 Hz), 7.22 (1H, t, J=7.32 Hz), 7.30 (2Hs, t, J=7.56 Hz), 8.1 (2Hs, d, J=9.52 Hz); ESI-MASS: 319.1 (M+23).

1-(4-Nitro-phenyl)-4-pyridin-2-yl-piperazine (26b). To a mixture of 4-fluoro nitro benzene 19b (425 mg, 3.01 mmol) and K₂CO₃ (623 mg, 4.52 mmol) in dimethyl sulfoxide (3 mL) was added 1-pyridin-2-yl-piperazine (0.91 mL, 6.02 mmol) and the reaction continued as described above to afford amine 770 mg of 26b in 90% yields. ¹H-NMR (500 MHz, CDCl₃): δ 3.6 (4Hs, t, J=5.12 Hz), 3.76 (4Hs, t, J=5.61 Hz), 6.65-6.72 (2Hs, m), 6.86 (2Hs, d, J=9.52 Hz), 7.54 (1H, dt, J=1.95, 7.07 Hz), 8.16 (2Hs, d, J=9.5 Hz), 8.22-8.25 (1H, m); ESI-MASS: 285.5 (M+1).

4-(3-Nitro-phenyl)-morpholine (27b). To a mixture of 3-fluoro nitro benzene 20b (425 mg, 3.01 mmol) and K₂CO₃ (623 mg, 4.52 mmol) in dimethyl sulfoxide (3 mL) was added morpholine (0.52 mL, 6.02 mmol) and the reaction continued as described above to afford amine 589 mg of 27b in 94% yields. ¹H-NMR (300 MHz, CDCl₃): δ 3.27 (4Hs, t, J=4.83 Hz), 3.9 (4Hs, t, J=4.95 Hz), 7.21 (1H, ddd, J=1.03, 2.25, 9.06 Hz), 7.42 (1H, t, J=8.19 Hz), 7.68-7.76 (2Hs m); ESI-MASS: 231.0 (M+23).

1-Methyl-4-(3-nitro-phenyl)-piperazine (28b). To a mixture of 3-fluoro nitro benzene 20b (425 mg, 3.01 mmol) and K₂CO₃ (623 mg, 4.52 mmol) in dimethyl sulfoxide (3 mL) was added 1-methyl piperazine (0.66 mL, 6.02 mmol) and the reaction continued as described above to afford 619 mg of amine 28b in 93% yields. ¹H-NMR (300 MHz, CDCl₃): δ 2.38 (3Hs, s), 2.6 (4Hs, t, J=5.0 Hz), 3.32 (4Hs, t, J=5.14 Hz), 7.2 (1H, dd, J=2.16, 8.26 Hz), 7.39 (1H, t, J=8.14 Hz), 7.67 (1H, dd, J=1.43, 8.01 Hz); ESI-MASS: 222.4 (M+1).

1-Benzyl-4-(3-nitro-phenyl)-piperazine (29b). To a mixture of 3-fluoro nitro benzene 20b (425 mg, 3.01 mmol) and K₂CO₃ (623 mg, 4.52 mmol) in dimethyl sulfoxide (3 mL) was added 1-benzyl piperazine (1.04 mL, 6.02 mmol) and the reaction continued as described above to afford amine 805 mg of 29b in 90% yields. ¹H-NMR (300 MHz, CDCl₃): δ 2.64 (4Hs, t, J=5.0 Hz), 3.31 (4Hs, t, J=5.14 Hz), 3.6 (2Hs, s), 7.18 (1H, dd, J=1.95, 8.33 Hz), 7.26-7.42 (6Hs, m), 7.66 (1H, ddd, J=0.63, 2.02, 8.0 Hz), 7.72 (1H, t, J=2.32 Hz); ESI-MASS: 298.1(M+1).

4-Benzyl-1-(3-nitro-phenyl)-piperidine (30b). To a mixture of 3-fluoro nitro benzene 20b (425 mg, 3.01 mmol) and K₂CO₃ (623 mg, 4.52 mmol) in dmethyl sulfoxide (3 mL) was added 4-benzyl piperidine (1.06 mL, 6.02 mmol) and the reaction continued as described above to afford amine 811 mg of 30b in 91% yields. ¹H-NMR (300 MHz, CDCl₃): δ 1.4 (2Hs, dq, J=4.14, 12.82, 24.75 Hz), 1.63-1.88 (3Hs, m), 2.61 (2Hs, d, J=6.72 Hz), 2.78 (2Hs, dt, J=2.53, 12.5 Hz), 3.77 (2Hs, d, J=12.53 Hz), 7.15-7.39 (7Hs, m), 7.62 (1H, dd, J=1.59, 7.98 Hz), 7.71 (1H, t, J=2.31); ESI-MIASS: 319.1 (M+23).

1-(3-Nitro-phenyl)-4-pyridin-2-yl-piperazine (31 b). To a mixture of 3-fluoro nitro benzene 20b (425 mg, 3.01 mmol) and K₂CO₃ (623 mg, 4.52 mmol) in. dimethyl sulfoxide (3 mL) was added 1-pyridin-2-yl-piperazine (0.91 mL, 6.02 mmol) and the reaction continued as described above to afford amine 770 mg of 31b in 90% yields. ¹H-NMR (300 MHz, CDCl₃): δ 3.43 (4Hs, t, J=5.41 Hz), 3.76 (4Hs, t, J=5.38 Hz), 6.67-6.78 (2Hs, m), 7.24 (1H, ddd, J=0.7, 2.49, 8.33 Hz), 7.42 (1H, t, J=8.14 Hz), 7.55 (1H, ddd, J=1.99, 7.16, 8.64 Hz), 7.7 (1H, ddd, J=0.81, 2.09, 8.04 Hz), 7.78 (1H, t, J=2.29 Hz), 8.24 (1H, ddd, J=0.9, 1.95, 4.91 Hz); ESI-MASS: 285.2 (M+1).

4-Morpholin-4-yl-benzonitrile (44b). To a mixture of 4-fluoro-benzonitrile 42b (1.0 g, 8.25 mmol) and K₂CO₃ (2.27 mg, 16.51 mmol) in dimethyl sulfoxide (7 mL) was added morpholine (1.07 mL, 12.38 mmol) and the reaction continued as described above to afford amine 1.38 g of 44b in 89% yields. ¹H-NMR (500 MHz, CDCl₃): δ 3.32 (4Hs, t, J=4.88 Hz), 3.89 (4Hs, t, J=4.88 Hz), 6.91 (2Hs, d, J=8.78 Hz), 7.54 (2Hs, d, J=8.78 Hz); ESI-MASS: 189.2 (M+1).

4-(4-Methyl-piperazin-1-yl)-benzonitrile (45b). To a mixture of 4-fluoro-benzonitrile 42b (1.0 g, 8.25 mmol) and K₂CO₃ (2.27 mg, 16.51 mmol) in dimethyl sulfoxide (7 mL) was added 1-methyl piperazine (1.36 mL, 12.38 mmol) and the reaction continued as described above to afford amine 1.54 g of 45b in 93% yields. ¹H-NMR (500 MHz, CDCl₃): δ 2.36 (3Hs, s), 2.55 (4Hs, t, J=4.88 Hz), 3.35 (4Hs, t, J=4.88 Hz), 6.87 (2Hs, d, J=8.78 Hz), 7.49 (2Hs, d, J=8.78 Hz); ESI-MASS: 202.1 (M+1).

4-(4-Benzyl-piperazin-1-yl)-benzonitrile (46b). To a mixture of 4-fluoro-benzonitrile 42b (1.0 g, 8.25 mmol) and K₂CO₃ (2.27 mg, 16.51 mmol) in dimethyl sulfoxide (7 mL) was added 1-benzyl piperazine (2.14 mL, 12.38 mmol) and the reaction continued as described above to afford amine 2.08 g of 46b in 91% yields. ¹H-NMR (500 MHz, CDCl₃): δ 2.58-2.68 (4Hs, bs),3.33-3.42 (4Hs, bs), 3.61 (3Hs, s), 6.88 (2Hs, d, J=8.54 Hz), 7.31-7.44 (5Hs, m), 7.52 (2Hs, d, J=8.54 Hz); ESI-MASS: 278.2 (M+1).

4-(4-Benzyl-piperidin-1-yl)-benzonitrile (47b). To a mixture of 4-fluoro-benzonitrile 42b (1.0 g, 8.25 mmol) and K₂CO₃ (2.27 mg, 16.51 mmol) in dimethyl sulfoxide (7 mL) was added morpholine (2.17 mL, 12.38 mmol) and the reaction continued as described above to afford amine 2.18 g of 47b in. 96% yields. ¹H-NMR (500 MHz, CDCl₃): δ 1.37 (2Hs, dq, J=3.9, 12.93, 25.14 Hz), 1.69-1.84 (3Hs, m), 2.62 (2Hs, d, J=6.83 Hz), 2.85 (2Hs, dt, J=2.19, 12.69 Hz), 3.87 (2Hs, d, J=13.18 Hz), 6.87 (2Hs, d, J=9.03 Hz), 7.2 (2Hs, d, J=8.05 Hz), 7.26 (1H, t, J=7.56 Hz), 7.34 (2Hs, t, J=7.56 Hz), 7.5 (2Hs, t, J=9.03 Hz); ESI-MASS: 299.7 (M+1).

3-(4-Methyl-piperazin-1-yl)-benzonitrile (48b). To a mixture of 3-fluoro-benzonitrile 43b (1.0 g, 8.25 mmol) and K₂CO₃ (2.27 mg, 16.51 mmol) in dimethyl sulfoxide (7 mL) was added 1-methyl piperazine (1.36 mL, 12.38 mmol) and the reaction continued as described above to afford amine 1.37 g of 48b in. 83% yields. ¹H-NMR (500 MHz, CDCl₃): δ 2.42 (3Hs, s), 2.63 (4Hs, t, J=4.88 Hz), 3.29 (4Hs, t, J=4.88 Hz), 7.13-7.18 (3Hs, m), 7.37 (1H, dd, J=7.56, 9.27 Hz); ESI-MASS: 202.2 (M+1).

3-(4-Benzyl-piperazin-1-yl)-benzonitrile (49b). To a mixture of 3-fluoro-benzonitrile 43b (1.0 g, 8.25 mmol) and K₂CO₃ (2.27 mg, 16.51 mmol) in dimethyl sulfoxide (7 mL) was added 1-benzyl piperazine (2.14 mL, 12.38 mmol) and the reaction continued as described above to afford amine 199 g of 49b in 87% yields. ¹H-NMR (500 MHz, CDCl₃): δ 2.65 (4Hs, t, J=5.12 Hz), 3.27 (4Hs, t, J=5.12 Hz), 3.61 (2Hs, s), 7.11-7.15 (3Hs, m), 7.31-7.41 (6Hs, m); ESI-MASS: 300.5 (M+23).

3-(4-Benzyl-peperidin-1-yl)-benzonitrile (50b). To a mixture of 3-fluoro-benzonitrile 43b (1.0 g, 8.25 mmol) and K₂CO₃ (2.27 mg, 16.51 mmol) in dimethyl sulfoxide (7 mL) was added 1-methyl piperazine (2.17 mL, 12.38 mmol) and the reaction continued as described above to afford amine 2.05 g of 50b in 90% yields. ¹H-NMR (500 MHz, CDCl₃): δ 1.43 (2Hs, dq, J=3.9, 12.45, 23.43 Hz), 1.73-1.86 (3Hs, m), 2.65 (2Hs, d, J=6.83 Hz), 2.88 (2Hs, dt, J=2.68, 12.45 Hz), 3.73 (2Hs, d, J=12.45 Hz), 7.1 (1H, td, J=0.97, 7.56 Hz), 7.14-7.17 (2Hs, m), 7.22 (2Hs, d, J=6.83 Hz), 7.27 (1H, t, J=7.32 Hz), 7.34-7.3 8 (3Hs, m); ESI-MASS: 299.5 (M+23).

General procedure for preparation of 32b -39b. To the substituted nitro compound (1 eq) in mixture of solvents methanol-ethyl acetate (1:2) was added 10% Pd-carbon (5% w/w) and subjected to hydrogenation under 50 Psi hydrogen gas pressure at room temperature. The reaction was monitored by TLC, after completion of reaction the reaction mix was filtered thorough celite bed and concentrated in vacuum to afford pure product in quantitative yields.

4-Morpholin-4-yl-phenylamine (32b). The 4-(4-Nitro-phenyl)-morpholine 22b, (600 mg, 2.88 mmol) in a mixture of solvents methanol-ethyl acetate (1:2, 20 mL) was treated with 10% Pd-carbon (5% w/w) under the conditions as described above to afford amine 32b in quantitative yield. The obtained product was used in the next reaction without further purification and characterization except mass-spectrometric analysis, ESI-MASS: 179.1 (M+1).

4-(4-Methyl-piperazin-1-yl)-phenylamine (33b). The 1-Methyl-4-(4-nitro-phenyl)-piperazine 23b, (600 mg, 2.71 mmol) in mixture of solvents methanol-ethyl acetate (1:2, 20 mL) was treated with 10% Pd-carbon (5% w/w) under the conditions as described above to afford amine 33b in quantitative yield. ¹H-NMR (500 MHz, CD₃OD): δ 2.41 (3Hs, s), 2.68 (4Hs, t, J=4.63 Hz), 3.08-3.13 (4Hs, bs), 3.33 (2Hs, q, J=1.70, 3.17 Hz), 6.73 (2Hs, d, J=8.3 Hz), 6.86 (2Hs, d, J=8.54 Hz); ESI-MASS: 192.2 (M+1).

4-(4-Benzyl-piperidin-1-yl)-phenylamine (34b). The 4-Benzyl-1-(4-nitro-phenyl)-piperidine 25b, (600 mg, 2.02 mmol) in mixture of solvents methanol-ethyl acetate (1:2, 20 mL) was treated with 10% Pd-carbon (5% w/w) under the conditions as described above to afford amine 34b in. quantitative yield. ¹H-NMR (500 MHz, CD₃OD). δ 1.44 (2Hs, q, J=9.27, 21.23 Hz), 1.6-1.7 (1H, m), 1.75 (1H, d, J=12.2 Hz), 2.55 (2Hs, t, J=11.47 Hz), 2.6 (2Hs, d, J=7.08 Hz), 3.39 (2Hs, d, J=11.23. Hz), 6.71 (2Hs, d, J=7.81 Hz), 6.87 (2Hs, d, J=8.05 Hz), 7.16-7.21 (3Hs, m), 7.28 (2Hs, t, J=7.07 Hz); ESI-MASS:267.1 (M+1).

4-(4-Pyridin-2-yl-piperazin-1-yl)-phenylamine (35b). The 1-(4-Nitro-phenyl)-4-pyridin-2-yl-piperazine 26b, (600 mg, 2.11 mmol) in mixture of solvents methanol-ethyl acetate (1:2, 20 mL) was treated with 10% Pd-carbon (5% w/w) under the conditions as described above to afford amine 35b in quantitative yield. ¹H-NMR (500 MHz, CDCl₃): δ 3.14 (4Hs, t, J=4.88 Hz), 3.68 (4Hs, t, J=5.12 Hz), 6.62-6.72 (4Hs, m), 6.86 (2Hs, d, J=8.78 Hz), 7.5 (1H, ddd, J=2.19, 7.32, 9.03 Hz), 8.21 (1H, dd, J=1.95, 4.15); ESI-MASS: 255.2 (M+1).

3-Morpholin-4-yl-phenylamine (36b). The 4-(3-Nitro-phenyl)-morpholine 27b, (600 mg, 2.88 mmol) in mixture of solvents methanol-ethyl acetate (1:2, 20 mL) was treated with 10% Pd-carbon (5% w/w) under the conditions as described above to afford amine 36b in quantitative yield. ¹H-NMR (300MHz, CDCl₃): δ 3.145 (4Hs, t, J=4.79 Hz), 3.86 (4Hs, t, J=4.89 Hz), 6.24-6.29 (2Hs, m), 6.37 (1H, ddd, J=0.73, 2.21, 8.20 Hz), 7.08 (1H, t, J=8.28); ESI-MASS: 201.3 (M+23).

3-(4-Methyl-piperazin-1-yl)-phenylamine (37b). The 1-Methyl-4-(3-nitro-phenyl)-piperazine 28b, (600 mg, 2.71 mmol) in mixture of solvents methanol-ethyl acetate (1:2, 20 mL) was treated with 10% Pd-carbon (5% w/w) under the conditions as described above to afford amine 37b in quantitative yield. ¹H-NMR (300 MHz, CDCl₃):δ 2.36 (3Hs, s), 2.57 (4Hs, t, J=4.94 Hz), 3.2 (4Hs, t, J=5.11 Hz), 3.59-3.67 (2Hs, bs), 6.22 (1H, dd, J=2.07, 8.41 Hz), 6.28 (1H, t, J=2.21 Hz), 6.39 (1H, dd, J=1.89, 7.82 Hz), 7.06 (1H, t, J=8.02 Hz); ESI-MASS: 192.1 (M+1).

3-(4-Benzyl-piperidin-1-yl)-phenylamine (38b). The 4-Benzyl-1-(3-nitro-phenyl)-piperidine 30b, (600 mg, 2.02 mmol) in mixture of solvents methanol-ethyl acetate (1:2, 20 mL) was treated with 10% Pd-carbon (5% w/w) under the conditions as described above to afford amine 38b in quantitative yield. ¹H-N7MIR (500 MHz, CDCl₃): δ 1.4 (2Hs, ddd, J=4.15, 11.96, 23.92 Hz), 1.61-1.69 (3Hs, m), 2.48-2.64 (4Hs, m), 3.47-3.64 (4Hs, m), 6.18 (1H, dd, J=1.22, 7.81 Hz), 6.25-6.29 (1H, bs), 6.36 (1H, dd, J=1.46, 8.3 Hz), 7.02 (1H, dt, J=1.22, 8.3 Hz), 7.16-7.25 (3Hs, m), 7.29 (2Hs, t, J=6.59); ESI-MASS: 267.4 (M+1).

3-(4-Pyridin-2-yl-piperazin-1-yl)-phenylamine (39b). The 1-(3-Nitro-phenyl)-4-pyridin-2-yl-piperazine 31b, (600 mg, 2.11 mmol) in mixture of solvents methanol-ethyl acetate (1:2, 20 mL) was treated with 10% Pd-carbon (5% w/w) under the conditions as described above to afford amine 39b in. quantitative yield. ¹H-NMR (500 MHz, CDCl₃): δ 3.28 (4Hs, dd, J=3.41, 5.12 Hz), 3.5-3.8 (6Hs, m), 6.26(1H, ddd, J=0.73, 1.95, 7.81 Hz), 6.31(1H, d, J=2.19 Hz), 6.41 (1H, ddd, J=0.48, 2.19, 8.05 Hz), 6.65 (1H, ddd, J=0.73, 4.88, 7.07 Hz), 6.71 (1H, d, J=8.54 Hz), 7.07 (1H, dt, J=1.95, 8.05 Hz), 7.51 (1H, dt, J=1.95, 7.08 Hz), 8.09-8.22 (1H, m); ESI-MASS: 255.3 (M+1).

General procedure for preparation of 40b and 41b. To a solution of the substituted nitro benzene in ethyl acetate (10 mL/mmol) SnCl₂.H₂O (1.125 g/mmol) was added. The solution was refluxed for 2 h. The cooled solution was diluted with water and the pH was adjusted to 7-8 by addition of saturated NaHCO₃ solution. The aqueous phase was extracted with EtOAc (3×75 mL) and the combined organic extracts were thoroughly washed with brine and dried over MgSO₄. The products obtained after the removal of the solvent were used without further purification.

4-(4-Benzyl-piperazin-1-yl)-phenylamine (40b). To a solution of the 1-benzyl-4-(4-nitro-phenyl)-piperazine 24b (750 mg, 2.52 mmol) in ethyl acetate (30 mL) SnCl₂.H₂O (14.4 g, 63.9 mmol) was added. The reaction was carried out as described above to afford 552 mg of amine 40b in. 82% yield. ¹H-NMR (300 MHz, CDCl₃): δ 2.6-2.8 (4Hs, bs), 3.11 (4Hs, t, J=4.38 Hz), 3.64 (2Hs, s), 6.66 (2Hs, d, J=8.76 Hz), 6.82 (2Hs, d, J=8.76 Hz), 7.25-7.45 (5Hs, m); ESI-MASS: 268.2 (M+1).

3-(4-Benzyl-piperazin-1-yl)-phenylamine (41 b). To a solution of the 1-benzyl-4-(3-nitro-phenyl)-piperazine 29b (750 mg, 2.52 mmol) in ethyl acetate (30 mL) SnCl₂.H₂O (14.4 g, 63.9 mmol) was added. The reaction was carried out as described above to afford 539 mg of amine 41b in. 82% yield. ¹H-NMR (300 MHz, CDCl₃): δ 2.58-2.75 (4Hs, bs), 3.17-3.32 (4Hs, bs), 3.56-3.71 (4Hs, bs), 6.21-6.29 (2Hs, m), 6.37 (1H, dd, J=1.89, 8.15 Hz), 7.09 (1H, t, J=4.57 Hz), 7.25-7.47 (5Hs, m); ESI-MASS: 268.3 (M+1).

General procedure for reduction of nitriles 44b -50b. To a solution of substituted aryl nitrile (1 mmol) in THF (5 mL) at 0° C. was added 65% red-Al in toluene (3 mmol) was added drop wise with stirring under argon atmosphere. The reaction was stirred for 3 h. at room temperature. The reaction was quenched by adding 1 mL methanol drop wise at 0° C. followed by 1 mL water. The reaction mixture was filtered through a celite bed washed with THF and the combined fractions were concentrated under vacuum. The resulting crude mixture was used in further reaction without further purification and characterization.

General procedure for preparation of 21b. 5-Nitro-furan-2-carboxylic acid(942 mg, 6 mmol) in DCM (10 mL) was treated with oxalylchloride (1.04 mL, 12 mmol) followed by 2 drops of DMF and stirred at room temperature for 4 hrs. The reaction mix was concentrated in vacuum to obtain acid chloride and the crude was used in further reactions without purification and characterization.

General procedure for preparation of compounds 54-70. 5-Nitro-furan-2-carbonyl chloride (438 mg, 2.5 mmol) in CH₂Cl₂ (10 mL) was added to a mixture of amine (2.0 mmol) in Et₃N (1.04 mL, 7.5 mmol) and the mixture was stirred for 12 hrs at room temperature. Reaction was followed by TLC, after completion of reaction 100 mL of ethyl acetate was added and washed with saturated aq. NaHCO₃ (2×50 mL), water (2×50 mL) and brine (2×50 mL). The organic phase was dried over Na₂SO₄, filtered and concentrated followed by flash column purification provided corresponding amides.

5-Nitro-furan-2-carboxylic acid(4-morpholin-4-yl-phenyl)-amide (54). 5-Nitro-furan-2-carbonyl chloride (438 mg, 2.5 mmol) in CH₂Cl₂ (10 mL) was added to a mixture of amine 32 (356 mg, 2.0 mmol) in Et₃N (1.04 mL, 7.5 mmol) and the mixture was stirred for 12 hrs. at room temperature. Reaction was carried out as explained above to afford 526 mg of amide 54 in. 83% yields. ¹H-NMR (500 MHz, CDCl₃): δ 3.19 (4Hs, t, J=4.88 Hz), 3.90 (4Hs, t, J=4.21 Hz), 6.97 (2Hs, d, J=8.78 Hz), 7.37 (1H, d, J=3.90 Hz), 7.43 (1H, d, J=3.90 Hz), 7.67.60 (2Hs, d, J=8.78 Hz), 8.20 (1H, bs); ¹³C-NMR (300 MHz, CDCl₃): ppm 48.83, 66.27, 12.14, 115.56, 121.24, 128.25, 147.64, 148.53, 153.16; ESI-MASS: 318.8 (M+1).

5-Nitro-furan-2-carboxylic acid [4-(4-methyl-piperazin-1-yl)-phenyl]-amide (55). 5-Nitro-furan-2-carbonyl chloride (438 mg, 2.5 mmol) in CH₂Cl₂(10 mL) was added to a mixture of amine 33b (356 mg, 2.0 mmol) in Et₃N (1.04 mL, 7.5 mmol) and the mixture was stirred for 12 hrs at room temperature. Reaction was carried out as explained above to afford 593 mg of amide 55 in 90% yields. ¹H-NMR (500 MHz, CDCl₃): δ2.39 (3Hs, s), 2.62 (4Hs, t, J=4.63 Hz), 3.25 (4Hs, t, J=5.12 Hz), 6.96 (2Hs, d, J=9.0 Hz), 7.37 (1H, d, J=3.90), 7.43 (1H, d, J=3.90), 7.57 (2Hs, d, J=9.0 Hz), 8.13 (1H, bs); ¹³C-NMR (300 MHz, CDCl₃): ppm 45.56, 48.52, 54.47, 112.14, 115.81, 121.18, 127.83, 147.68, 148.56, 153.118; ESI-MASS: 331.6 (M+1).

5-Nitro-furan-2-carboxylic acid [4-(4-benzyl-piperazin-1-yl)-phenyl]-amide (56). 5-Nitro-furan-2-carbonyl chloride (438 mg, 2.5 mmol) in CH₂Cl₂ (10 mL) was added to a mixture of amine 40b (534 mg, 2.0 mmol) in Et₃N (1.04 mL, 7.5 mmol) and the mixture was stirred for 12 hrs at room temperature. Reaction was carried out as explained above to afford 755 mg of amide 56 in 93% yields. ¹H-NMR (500 MHz, CDCl₃): δ2.66-2.76 (4Hs, bs), 3.26-3.33 (4Hs, bs), 3.64-3.7 (2Hs, bs), 7.99 (2Hs, d, J=9.03 Hz), 7.32-7.37 (2Hs, m), 7.3 8-7.46 (4Hs, m), 7.47 (1H, d, J=3.90), 7.61 (2Hs, d, J=9.03 Hz), 8.14 (1H, bs); ¹³C-NMR (300 MHz, CDCl₃-DMSO-D₆, 5:1): ppm 48.13, 51.98, 61.85, 111.94, 114.87, 114.91, 121.36, 126.12, 127.29, 128.12, 128.67, 137.17, 147.65, 148.11, 150.69, 153.39; ESI-MASS: 407.5 (M+1). Anal. Calcd. for C₂₂H₂₂N₄O₄: C, 65.01; H, 5.46; N, 13.78. Found: C, 64.94; H, 5.41; N, 13.64.

5-Nitro-furan-2-carboxylic acid [4-(4-benzyl-piperidin-1-y1 )-phenyl]-amide (57). 5-Nitro-furan-2-carbonyl chloride (438 mg, 2.5 mmol) in CH₂Cl₂ (10 mL) was added to a mixture of amine 34b (532 mg, 2.0 mmol) in Et₃N (1.04 mL, 7.5 mmol) and the mixture was stirred for 12 hrs at room temperature. Reaction was carried out as explained above to afford 728 mg of amide 57 in 90% yields. ¹H-NMR (500 MHz, CDCl₃): δ1.50-1.65 (3Hs, m), 1.65-1.85 (2Hs, m), 2.6-2.8 (4Hs, m), 3.68 (2Hs, d, J=10.98 Hz), 6.9-7.02 (2Hs, bs), 7.2-7.3 (3Hs, m), 7.3-7.39 (2Hs, m), 7.41 (1H, d, J=3.66), 7.47 (1H, t, J=2.1, 3.6), 7.515-7.65 (2Hs, bs), 8.12-8.22 (1H, bs); ¹³C-NMR 300 MHz, (CDCl₃): ppm 31.37, 37.26, 42.58, 49.36, 112.13, 115.74, 116.16, 121.15, 125.38, 127.34, 127.70, 128.59, 139.84, 147.74, 149.11, 153.06; ESI-MASS:406.4 (M+1).

5-Nitro-furan-2-carboxylic acid. [4-(4-pyridin-2-yl-piperazin-1-yl)-phenyl]-amide (58). 5-Nitro-furan-2-carbonyl chloride (438 mg, 2.5 mmol) in CH₂Cl₂ (10 mL) was added to a mixture of amine 35b (508 mg, 2.0 mmol) in Et₃N (1.04 mL, 7.5 mmol) and the mixture was stirred for 12 hrs at room temperature. Reaction was carried out as explained above to afford 723 mg of amide 58 in 92% yields. ¹H-NMR (500 MHz, CDCl₃): δ 3.37-3.42 (4Hs, bs), 3.75-3.88 (4Hs, bs), 6.54-6.65 (2Hs, bd), 7.02 (2Hs, d, J=9.03 Hz), 7.38 (1H, d, J=3.66 Hz), 7.44(1H, d, J=3.66 Hz), 7.61 (2Hs, d, J=9.04 Hz), 8.15(1H, bs), 8.29(1H, dd, J=1.46, 4.88 Hz); ¹³C-NMR (300 MHz, CDCl₃): ppm 44.69, 48.62, 106.69, 112.14, 113.11, 115.85, 116.14, 121.22, 128.16, 137.01, 147.50, 147.64, 148.55, 153.13, 158.85; ESI-MASS: 394.4 (M+1). Anal. Calcd. for C₂₀H₁₉N₅O₄: C, 61.06; H, 4.87; N, 17.80. Found: C, 60.77; H, 4.93; N, 17.57.

5-Nitro-furan-2-carboxylic acid(3-morpholin-4-yl-phenyl)-amide (59). 5-Nitro-furan-2-carbonyl chloride (438 mg, 2.5 mmol) in CH₂Cl₂ (10 mL) was added to a mixture of amine 36b (356 mg, 2.0 mmol) in Et₃N (1.04 mL, 7.5 mmol) and the mixture was stirred for 12 hrs at room temperature. Reaction was carried out as explained above to afford 494 mg of amide 59 in 78% yields. ¹H-NMR (500 MHz, CDCl₃): δ 3.23 (4Hs, t, J=4.63 Hz), 3.89 (4Hs, t, J=4.88 Hz), 6.77 (1H, dd, J=2.19, 8.3 Hz), 7.1 (1H, dd, J=1.46, 7.81 Hz), 7.30 (1H, t, J=8.05 Hz), 7.38 (1H, d, J=3.66 Hz), 7.46-7.5 (2Hs, m), 8.20 (1H, bs); ¹³C-NMR (300 MHz, CDCl₃): ppm 51.75, 69.37, 110.78, 114.99, 115.26, 115.31, 119.17, 132.16, 140.47, 150.80, 154.44, 157.39; ESI-MASS: 318.3 (M+1).

5-Nitro-furan-2-carboxylic acid [3-(4-methyl-piperazin-1-yl)-phenyl]-amide (60). 5-Nitro-furan-2-carbonyl chloride (438 mg, 2.5 mmol) in CH₂Cl₂ (10 mL) was added to a mixture of amine 37b (382 mg, 2.0 mmol) in Et₃N (1.04 mL, 7.5 mmol) and the mixture was stirred for 12 hrs at room temperature. Reaction was carried out as explained above to afford 593 mg of amide 60 in 90% yields. ¹H-NMR (500 MHz, CDCl₃): δ 2.38 (3Hs, s), 2.60 (4Hs, t, J=4.64 Hz), 3.28 (4Hs, t, J=4.51 Hz), 6.8 (1H, d, J=8.3 Hz), 7.08 (1H, d, J=8.05 Hz), 7.25-7.32 (2Hs, m), 7.37-7.45 (2Hs, m), 7.48 (1H, dd, J=1.22, 3.66 Hz), 8.17 (1H, bs); ¹³C-NMR (300 MHz, CD₃OD): ppm 44.16, 47.92, 53.98, 107.99, 111.48, 111.70, 112.26, 115.49, 128.50, 137.57, 147.55, 151.16, 154.73; ESI-MASS: 331.3 (M+1). Anal. Calcd. for C₁₆H₁₈N₄O₄: C, 58.17; H, 5.49; N, 16.96. Found: C, 57.94; H, 5.40; N, 16.92.

5-Nitro-furan-2-carboxylic acid [3-(4-benzyl-piperazin-1-yl)-phenyl]-amide (61). 5-Nitro-furan-2-carbonyl chloride (438 mg, 2.5 mmol) in CH₂Cl₂ (10 mL) was added to a mixture of amine 41b (534 mg, 2.0 mmol) in Et₃N (1.04 mL, 7.5 mmol) and the mixture was stirred for 12 hrs at room temperature. Reaction was carried out as explained above to afford 747 mg of amide 61 in 92% yields. ¹H-NMR (500 MHz, CDCl₃): δ2.6-2.74 (4Hs, bs), 3.26-3.36 (4Hs, bs), 3.59-3.7 (2Hs, bs), 6.76 (1H, dd, J=2.19, 8.05 Hz), 7.09 (1H, d, J=8.05 Hz), 7.25-7.35 (3Hs, m), 7.35-7.42 (4Hs, m), 7.43 (1H, d, J=3.66), 8.04 (1H, s), 8.2 15 (1H, s); ¹³C-NMR (300 MHz, CDCl₃): ppm 48.23, 52.41, 62.46, 107.31, 110.79, 112.16, 112.25, 116.02, 126.60, 127.73, 128.63, 129.09, 136.98, 137.38, 147.68, 150.72, 151.50, 153.52; ESI-MASS: 407.5 (M+1).

5-Nitro-furan-2-carboxylic acid [3-(4-benzyl-piperidin-1-yl)-phenyl]-amide (62). 5-Nitro-furan-2-carbonyl chloride (438 mg, 2.5 mmol) in CH₂Cl₂(10 mL) was added to a mixture of amine 38b (532 mg, 2.0 mmol) in Et₃N (1.04 mL, 7.5 mmol) and the mixture was stirred for 12 hrs at room temperature. Reaction was carried out as explained above to afford 777 mg of amide 62 in 96% yields. ¹H-NMR (500 MHz, CDCl₃): δ 1.50-1.67 (3Hs, m), 1.67-1.88 (2Hs, m), 2.63 (2Hs, d, J=6.84), 2.73-2.88 (2Hs, m), 3.78 (2Hs, d, J=12.20 Hz), 6.78-6.84 (1H, bs). 7.04-7.16 (1H, bs), 7.16-7.36 (8Hs, m), 7.38 (1H, d, J=3.9 Hz), 7.43 (1H, d, J=7 Hz), 8.15-8.28 (1H, bs); ¹³C-NMR (300 MHz, CDCl₃): ppm 31.30, 37.31, 42.58, 48.92, 76.70, 107.60, 110.38, 112.23, 112.70, 116.0, 125.40, 127.73, 128.63, 129.05, 136.96, 139.89, 147.66, 150.74, 151.81, 153.63; ESI-MASS: 406.4 (M+1).

5-Nitro-furan-2-carboxylic acid [3-(4-pyridin-2-yl-piperazin-1-yl)-phenyl]-amide (63). 5-Nitro-furan-2-carbonyl chloride (438 mg, 2.5 mmol) in CH₂Cl₂ (10 mL) was added to a mixture of amine 39b (508 mg, 2.0 mmol) in Et₃N (1.04 mL, 7.5 mmol) and the mixture was stirred for 12 hrs at room temperature. Reaction was carried out as explained above to afford 682 mg of amide 63 in 85% yields. ¹H-NMR (500 MHz, CDCl₃): δ 3.40 (4Hs, t, J=5.37 Hz), 3.72-3.82 (4Hs, bs), 6.71 (1H, t, J=5.85 Hz), 6.76 (1H, d, J=8.54), 6.83 (1H, dd, J=2.44, 8.05 Hz), 7.11(1H, dd, J=1.7, 7.56 Hz), 7.32 (1H, t, J=8.05 Hz), 7.4 (1H, d, J=3.90 Hz), 7.44(1H, d, J=3.66), 7.47(1H, t, J=2.19 Hz), 7.56-7.62 (1H, bs), 8.29 (1H, s), 8.25 (1H, dd, J=1.70, 4.88 Hz); ¹³C-NMR (300 MHz, CDCl₃): ppm 44.47, 48.02, 107.21, 107.85, 111.55, 112.13, 113.18, 113.38, 116.29, 129.17, 137.48, 138.54, 147.51, 148.01, 151.27, 151.64, 154.42, 158.90; ESI-MASS: 394.4 (M+1). Anal. Calcd. for C₂₀H₁₉N₅O₄: C, 61.06; H, 4.87; N, 17.80. Found: C, 61.09; H, 4.95; N, 17.60.

5-Nitro-furan-2-carboxylic acid 4-morpholin-4-yl-benzylamide (64). 5-Nitro-furan-2-carbonyl chloride (438 mg, 2.5 mmol) in CH₂Cl₂ (10 mL) was added to a mixture of crude amine 44b (384 mg, 2.0 mmol) in Et₃N (1.04 mL, 7.5 mmol) and the mixture was stirred for 12 hrs at room temperature. Reaction was carried out as explained above to afford 469 mg of amide 64 in 71% yields. ¹H-NMR (500 MHz, CDCl₃): δ 3.22 (4Hs, t, J=4.88 Hz), 3.92 (4Hs, t, J=4.88 Hz), 4.61 (2Hs, d, J=5.61 Hz), 6.78-6.82 (1H, bs), 6.96 (2Hs, d, J=8.78 Hz), 7.31-7.35 (3Hs,. m), 7.41 (1H, d, J=3.90 Hz); ¹³C-NMR (300 MHz, DMSO-D₆): ppm 41.81, 48.56, 65.99, 113.34, 114.99, 115.42, 128.39, 129.20, 148.29, 150.23, 151.37, 155.88; ESI-MASS: 332.4 (M+1); Anal. Calcd. for C₁₆H₁₇N₃O₅: C, 58.00; H, 5.17; N, 12.68. Found: C, 57.71;H, 5.23;N, 12.41.

5-Nitro-furan-2-carboxylic acid 4-(4-methyl -piperazin-1-yl)-benzylamide (65). 5-Nitro-furan-2-carbonyl chloride (438 mg, 2.5 mmol) in CH₂Cl₂ (10 mL) was added to a mixture of crude amine 45b (410 mg, 2.0 mmol.) in Et₃N (1.04 mL, 7.5 mmol) and the mixture was stirred for 12 hrs at room temperature. Reaction was carried out as explained above to afford 481 mg of amide 65 in 70% yields. ¹H-NMR (500 MHz, CDCl₃): δ 2.41 (3Hs, s), 2.63 (4Hs, t, J=4.88 Hz), 3.27 (4Hs, t, J=4.88 Hz), 4.6 (2Hs, d, J=5.61 Hz), 6.78-6.83 (1H, bs), 6.97 (2Hs, d, J=8.78 Hz), 7.31 (2Hs, d, J=8.78 Hz), 7.33 (1H, d, J=3.90 Hz), 7.41 (1H, d, J=3.90 Hz); ¹³C-NMR (300 MHz, CDCl₃): ppm 41.71, 44.02, 48.06, 53.94, 111.31, 114.88, 115.52, 127.89, 128.91, 147.54, 149.95, 156.61; ESI-MASS: 345.3 (M+1); Anal. Calcd. for C₁₇H₂ON₄O₄: C, 59.29; H, 5.85; N, 16.27. Found: C, 59.16; H, 5.91; N, 16.19.

5-Nitro-furan-2-carboxylic acid 4-(4-benzyl-piperazin-1-yl)-benzylamide (66). 5-Nitro-furan-2-carbonyl chloride (438 mg, 2.5 mmol) in CH₂Cl₂ (10 mL) was added to a mixture of crude amine 46b (562 mg, 2.0 mmol) in Et₃N (1.04 mL, 7.5 mmol) and the mixture was stirred for 12 hrs at room temperature. Reaction was carried out as explained above to afford 363 mg of amide 66 in 79% yields. ¹H-NMR (500 MHz, CDCl₃): a 2.66 (4Hs, t, .J=5.12 Hz), 3.26 (4Hs, t, J=5.12 Hz), 3.62 (3Hs, s), 4.60 (2Hs, d, J=5.61 Hz), 6.77-6.82 (1H, bs), 6.96 (2Hs, d, J=8.78 Hz), 7.30 (2Hs, d, J=8.54 Hz), 7.31-7.35 (4Hs, m), 7.37-7.43 (4Hs, m); ¹³C-NMR (300 MHz, CDCl₃): ppm 42.67, 48.40, 52.44, 62.50, 111.84, 115.39, 115.53, 126.62, 126.94, 127.74, 128.63, 128.65, 137.39, 147.60, 150.65, 155.46; ESI-MASS: 421.5 (M+1).

5-Nitro-furan-2-carboxylic acid 4-(4-benzyl-piperidin-1-yl)-benzylamide (67). 5-Nitro-furan-2-carbonyl chloride (438 mg, 2.5 mmol) in CH₂Cl₂ (10 mL) was added to a mixture of crude amine 47b (560 mg, 2.0 mmol) in Et₃N (1.04 mL, 7.5 mmol) and the mixture was stirred for 12 hrs at room temperature. Reaction was carried out as explained above to afford 720 mg of amide 67 in 86% yields. ¹H-NMR (500 MHz, CDCl₃): δ 1.46 (2Hs, dq, J=3.9, 12.44, 23.92 Hz), 1.69-1.83 (3Hs, m), 2.64 (2Hs, d, J=7.08 Hz), 2.72 (2Hs, dt, J=2.44, 12.2 Hz), 3.72 (2Hs, d, J=12.45 Hz), 4.58 (2Hs, d, J=5.85 Hz), 6.78-6.82 (1H, bs), 6.96 (2Hs, d, J=8.78 Hz), 7.2-7.3 (5Hs, m), 7.32 (1H, d, J=40 Hz), 7.35 (2Hs, t, J=7.32 Hz), 7.40 (1H, d, J=3.66 Hz); ¹³C-NMR (300 MHz, CDCl₃): ppm 31.35, 37.33, 42.60, 42.70, 49.19, 111.88, 115.38, 115.93, 125.39, 126.53, 127.71, 128.49, 128.60, 128.62, 139.86, 147.65, 150.98, 155.48; ESI-MASS: 420.6 (M+1).

5-Nitro-furan-2-carboxylic acid 3-(4-methyl-piperazin-1-yl)-benzylamide (68). 5-Nitro-furan-2-carbonyl chloride (438 mg, 2.5 mmol) in CH₂Cl₂ (10 mL) was added to a mixture of crude amine 48b (410 mg, 2.0 mmol) in Et₃N (1.04 mL, 7.5 mmol) and the mixture was stirred for 12 hrs at room temperature. Reaction was carried out as explained above to afford 474 mg of amide 68 in 69% yields. ¹H-NMR (500 MHz, CDCl₃): δ 2.41 (3Hs, s),2.64 (4Hs, t, J=4.88 Hz), 3.29 (4Hs, t, J=4.88 Hz), 4.63 (2Hs, d, J=5.85 Hz), 6.89 (2Hs, d, J=7.32). 6.93-6.98 (2Hs, m), 7.3-7.36 (2Hs, m), 7.42 (1H, d, J=3.66 Hs); ESI-MASS: 345.1 (M+1).

5-Nitro-furan-2-carboxylic acid 3-(4-benzyl-piperazin-1-yl)-benzylamide (69). 5-Nitro-furan-2-carbonyl chloride (438 mg, 2.5 mmol) in CH₂Cl₂ (10 mL) was added to a mixture of crude amine 49b (562 mg, 2.0 mmol) in Et₃N (1.04 mL, 7.5 mmol) and the mixture was stirred for 12 hrs at room temperature. Reaction was carried out as explained above to afford 604 mg of amide 69 in 72% yields. ¹H-NMR₁ (500 MHz, CDCl₃): δ 2.66 (4Hs, t,. J=4.88 Hz), 3.272 (4Hs, t, J=5.12 Hz), 3.62 (2Hs, s), 4.63 (2Hs, d, J=5.85 Hz), 6.87 (1H, d, J=7.56 Hz), 6.9-6.95 (3Hs, m), 7.29-7.35 (3Hs, m), 3.7-3.5 (5Hs, m); ESI-MASS: 421.5 (M+1).

5-Nitro-furan-2-carboxylic acid 3-(4-benzyl-piperidin-1-yl)-benzylamide (70). 5-Nitro-furan-2-carbonyl chloride (438 mg, 2.5 mmol) in CH₂Cl₂ (10 mL) was added to a mixture of crude amine 50b (560 mg, 2.0 mmol) in Et₃N (1.04 mL, 7.5 mmol) and the mixture was stirred for 12 hrs at room temperature. Reaction was carried out as explained above to afford 695 mg of amide 70 in 83% yields. ¹H-NMR (500 MHz, CDCl₃): δ1.44 (2Hs, dq, J=3.66, 11.71, 23.92 Hz), 1.68-1.77 (1H, m), 1.78 (2Hs, d, J=13.18 Hz), 2.62 (2Hs, d, J=2.83 Hz), 2.7 (2Hs, dt, J=2.19, 12.20 Hz), 3.72 (2Hs, d, J=12.45 Hz), 4.61 (2Hs, d, J=5.61 Hz), 6.81-6.88 (2Hs, m), 6.9-6.94 (2Hs, m), 7.21 (2Hs, d, J=7.07 Hz), 7.22-7.36 (5Hs, m), 7.40 (1H, d, J=3.66 Hz); ¹³C-NMR (300 MHz, CDCl₃): ppm 31.41, 37.30, 42.57, 43.54, 49.17, 111.84, 115.27, 115.48, 118.09, 125.28, 127.70, 128.59, 129.10, 137.20, 139.85, 147.52, 151.74, 155.52; ESI-MASS: 420.4 (M+1); Anal. Calcd. for C₂₄H₂₅N₃O₄: C, 68.72, H, 6.01; N, 10.02. Found: C, 68.33; H, 6.01; N, 10.02.

### Biological Results

The MIC of the nitrofuranyl amides against M tuberculosis H37Ra was determined by the micro broth dilution method using microtiter plates. M. tuberculosis was grown in Middlebrook 7H9 medium to an OD₆₅₀ of 0.4-0.6 and a dilution made to an. OD₆₅₀ of 0.01. 100 μl of these cells are then added to a microtiter well containing serial dilutions of the nitrofuranyl amides. The cells are then incubated at 37° C. for 7 days and visually examined for growth. MIC₉₀ was determined for wells with greater than 90% inhibition of growth. Results are shown in Table 4 below.

The synthesis of cyclic, tertiary amine substituted, benzyl nitrofuranyl amides required the preparation of amine ring building blocks, which were synthesized by nucleophilic displacement of nitro or cyano phenyl fluorides with the selected secondary amine, followed by reduction of the cyano or nitro group to produce the desired benzylamine and phenylamines respectively. The remaining amides targeted in this series of compounds were synthesized by acylation of the corresponding amine using our standard procedure from Example 1. Thus, a set of: 8 benzyl nitrofuranyl amides; 3 fused tertiary benzyl nitrofurariyl amides; 18 cyclic tertiary amine substituted benzyl and phenyl nitrofuranyl amides; and 19 benzyl or phenyl piperazinyl nitrofuranyl amides was designed and synthesized.

The choice of substitution in this series was based on the first series of compounds in Example 1 and on the developing knowledge of the SAR of the series. Some of the most active second generation compounds in this series are shown in Table 4. More interesting SAR was developed in this series: (i) constrained tertiary amides, such as compound 71, have increased activity over N-methyl aniline amide 41 (Example 1; MIC 3.1 μg/ml) (ii) benzyl piperazine 73, and piperidine amide 72, had good activity compared to N-methyl piperazine 76 (MIC 37.5 μg/ml) (iii) tertiary amide 75, an analog of the in vivo active compound 23, was very potent (iv) increasing the solubility of the benzyl nitrofuranyl amide series by substituting the benzyl ring with a cyclic amine, such as compound 66, also increased the potency against M. tuberculosis, suggesting that in addition to increasing the solubility, these substitutions may also affect the bacterial uptake.

Example 2 describes the synthesis of the target molecules in good yields. As can be seen by the data, no barrier to scale up synthesis for larger quantities for in vivo testing is offered by these synthesis schemes. Therefore, in vivo testing using the techniques disclosed herein, along with general knowledge and skills presently available in the art can be readily achieved by one of ordinary skill in the art.

There is a clear structure activity relationship for the compounds described in Example 2, with the substituted benzyl compounds having greater anti-tuberculosis than the substituted phenyl compounds (see Table 4 below). In both the phenyl and the benzyl amides para-substitution with the cyclic secondary amine produced better anti-tuberculosis activity. Compounds 66 and 70, both from the benzyl series, are extremely potent and are the most active compounds so far developed in this class.

TABLE 4
Cyclic secondary amine substituted phenyl and benzyl
nitrofuranyl amides and their, anti-tuberculosis activity

| No. | Compound | MIC (μg/mL) |
|---|---|---|
| 54 | 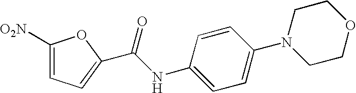 | 1.6 |
| 55 | 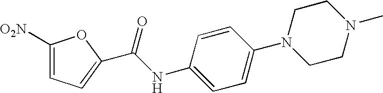 | 6.25 |
| 56 | 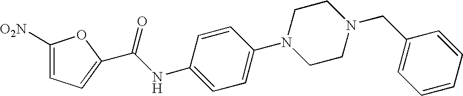 | 0.8 |
| 57 | 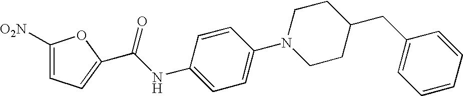 | 3.1 |
| 58 | 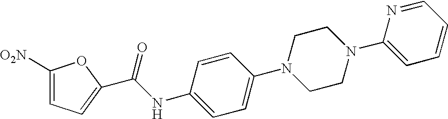 | 0.4-0.8 |
| 59 | 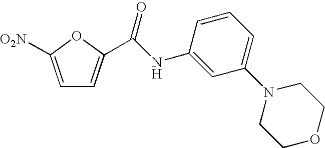 | 3.1 |
| 60 | 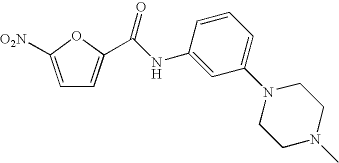 | 12.5 |
| 61 | 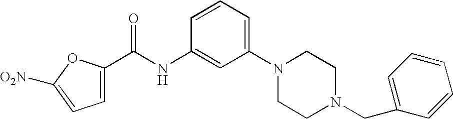 | 12.5 |
| 62 | 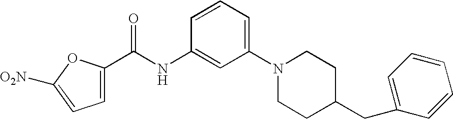 | 12.5 |
| 63 | 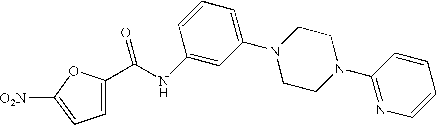 | 1.6 |
| 64 | 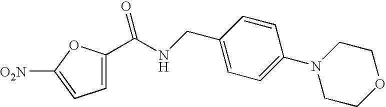 | 0.1 |
| 65 | 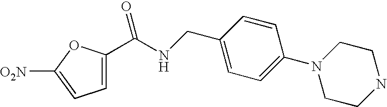 | 0.1 |
| 66 | 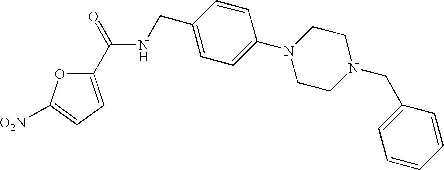 | 0.0062 |
| 67 | 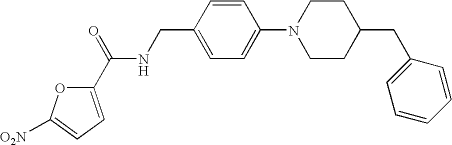 | <0.1 |
| 68 | 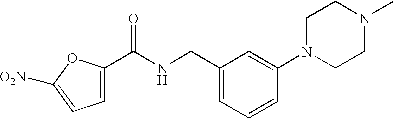 | 3.1 |
| 69 | 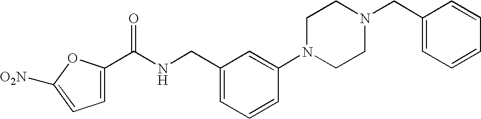 | <0.1 |
| 70 | 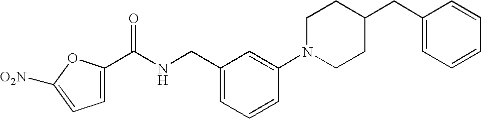 | 0.0062 |
| 71 | 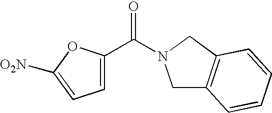 | 0.78 |

Comparison of Example 2 Lead Compounds with other Antimycobacterial Drugs.

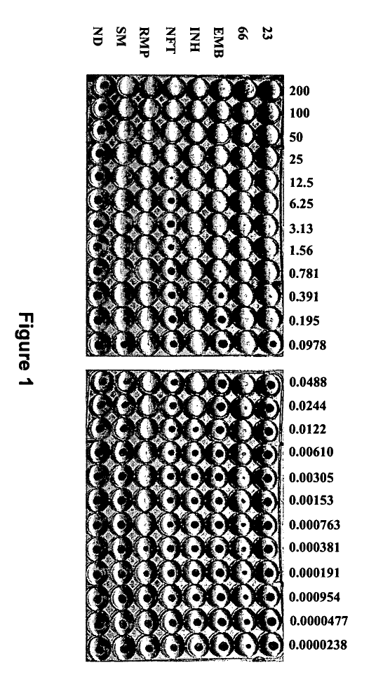

Two nitrofuranyl amides, one from each round of optimization, were assayed for MIC activity against M. tuberculosis H37Ra, along with nitrofurantoin (NFT) and 4 classical TB drugs: ethambutol (EMB), isoniazid (INH), Rifampin (RMP), and streptomycin sulfate (SM) (FIG. 1). The MIC values for the TB drugs and nitrofurantoin are similar to those in the literature for M. tuberculosis (R. E. Lee et al., J. Combinatorial Chem. 2003, 5(2), 172-87). Against H37Ra, compound 23 is as active as ethambutol and streptomycin, and compound 66 is more active than isoniazid, and perhaps even as active as rifampin. It appears that compound 66 has a sub MIC growth impairment effect, although in the virulent strain, H37Rv, the effect is much less pronounced. The MIC activities of these novel compounds in the ng/ml range are very encouraging.

### Discussion of Examples 1 and 2

There are structural differences between the nitrofuranyl amides disclosed herein, and particularly in Examples 1 and 2 and the nitrofuranyl imine antibiotics such as nitrofurantoin: (i) the amide bond is more electron withdrawing than the imine linkage, which will alter the reactivity of the nitrofuran group to metabolic activation; (ii) the amide and imine linkages will be metabolized differently, and the introduction of the amide bond can introduce a desirable site for secondary metabolism; (iii) nitrofuranyl imine antibiotics are bicyclic and the nitrofuranyl amides are mostly tricyclic, including one unsaturated ring; and (iv) the most active compounds in the nitrofuranyl amide series are 10,000-100,000-fold more active against M. tuberculosis than any of the clinically prescribed nitrofuranylimine antibiotics, suggesting non-bioequivalence.

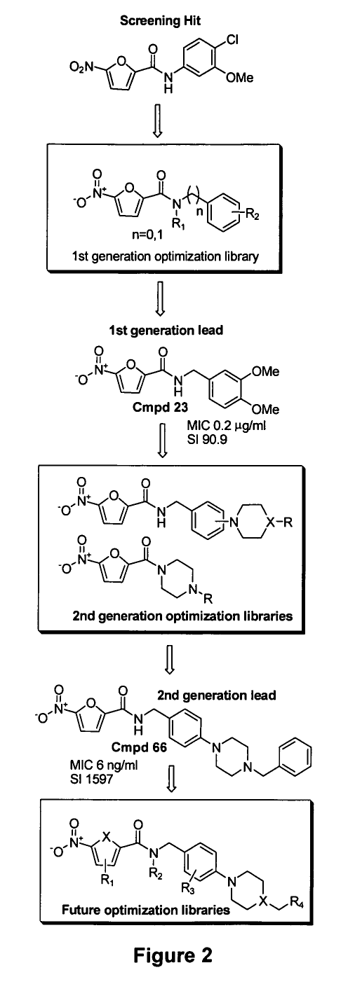

In summary, Examples 1 and 2 discuss two rounds of lead optimization based on in vitro testing that produced potent compounds (see FIG. 2). From the discovery of the first screening hit, an optimization library of 43 compounds was synthesized (Example 1), from which compound 23 (MIC 0.2mg/mL and selectivity index (SI) 90.9) was selected as a lead compound for further optimization. A second-generation optimization library was synthesized (Example 2), following previously successful antimicrobial design strategies to improve the bioavailability and solubility of the series. These changes also increased the activity against M. tuberculosis yielding second-generation lead compound 66 (MIC 6 ng/ml, SI 1597). Compounds in this generation were more soluble and better formulated.

The compounds disclosed herein can be further optimized based on in vivo testing and toxicology data to achieve increased bioavailability and in vivo activity. The metabolism and mode of action of these and further compounds can also be studied, as is known in the art. Importantly, this compound series has a number of promising features that makes them attractive new anti-tuberculosis agents: they possess extremely potent MIC values; they have good selectivity indexes; activity has been demonstrated in an in vivo model of tuberculosis infection; the MIC activity of this series is comparable to front-line anti-tuberculosis agents such as isoniazid and ethambutol; the compounds are not cross-resistant with other clinically used anti-tuberculosis agents; the compounds can be easily synthesized at low cost; and lastly, the compounds are novel.

### Example 3

Examples 1 and 2 describe developing compounds with potent anti-tuberculosis activity, with at least 7 compounds with MIC values in the 5-100 ng/mL range. This Example pertains to developing additional compounds and focuses on improving the solubility and bioavailability of the series. Without wishing to be limited by theory, limited bioavailability can be a result of 3 factors: (i) the metabolic instability of the amide; (ii) the solubility of compounds in this class; (iii) high serum binding and poor tissue distribution.

To address the first issue, a number of tertiary amides can be tested and alternative linkages which should have increased stability to proteolysis can be explored. Increasing the solubility of compounds in this series was addressed in Example 2 above by adding an ionizable or polar side chain in the form of a substituted piperazine or morpholine rings, a strategy that has been successfully used to develop oral bioavailability in other antimicrobial agents. This strategy led to the successful representative second generation compound 66, discussed above. As this strategy was clearly successful, continued expansion of the series based on compound 66 is discussed in this example, along with careful monitoring of changes in SAR, and how they influence the predicted and observed bioavailability. Modifying the leads to alter serum binding and tissue distribution can be addressed by testing functional group substitutions that are known to decrease protein binding and through further analysis of the results discussed above.

### Scheme 1

In the second generation optimization library discussed in Example 2, 18 cyclic tertiary amine substituted at 3 and 4 positions of benzyl (8 compounds) and phenyl (10 compounds) nitrofuranyl amides were synthesized. As the 4-benzyl series showed significantly superior activity, especially compound 66, this series is further elaborated upon, as shown in Scheme 1 below. The previous compounds were synthesized starting from 3 or4-fluorobenzonitrile, which was reacted with 4 different substituted piperazines in a nucleophilic aromatic displacement reaction. The nitriles were then reduced and acylated to afford the target compounds in good yields. The previous series is expanded through two major synthesis routes (Scheme 1). Route 1 uses a convenient commercially available starting material that contains both B and C rings and allows for substitution of the piperazine ring prior to reduction of the nitrile. The piperazine ring can be further elaborated by reductive amination using a wide range of commercially available aldehydes and especially important for the developing SAR substituted benzaldehydes. Substitutions to the piperazine ring by alkylation with a variety alkylhalides such as bromomethylcyclopropane can be further explored. Both these elaborations are ideal chemistries for parallel synthesis and should offer no significant synthetic challenges.

Route 2 utilizes nucleophilic aromatic displacement to introduce new B and C rings. Substitutions to the benzyl B ring can be carried out using other commercially available trisubstituted 4-fluorobenzonitriles such as 3,4-difluorobenzonitrile. As the 4-position is the most activated the previously used conditions can be applied to synthesize substituted 4-piperazinyl benzyl amides and to evaluate the effects of having a halide substituted benzyl B ring. A number of other cyclic amine substitutions can be tested using novel building blocks to study their effects on the bioavailability of this series. There are a large number of potential cyclic amines commercially available for purchase from commercial entities as is known to one of skill in the art, such as, for example, Maybridge, Maybridge, United Kingdom and Sigma-Aldrich, St. Louis, Mo., USA.

One area of consideration that factors into the third generation design is the high predicted protein binding values for compound 66 and therefore it is desirable to evaluate compounds with lower protein binding numbers. Thus, each proposed compound can be modeled before starting synthesis to ensure that it has appropriate drug like physical properties. One compound which can be targeted for the third generation series is the thiomorpholine analog, which upon completion of synthesis to the nitrofuranyl amide, can be further oxidized to the corresponding cyclic sulfone, a substitution that typically decreases plasma binding.

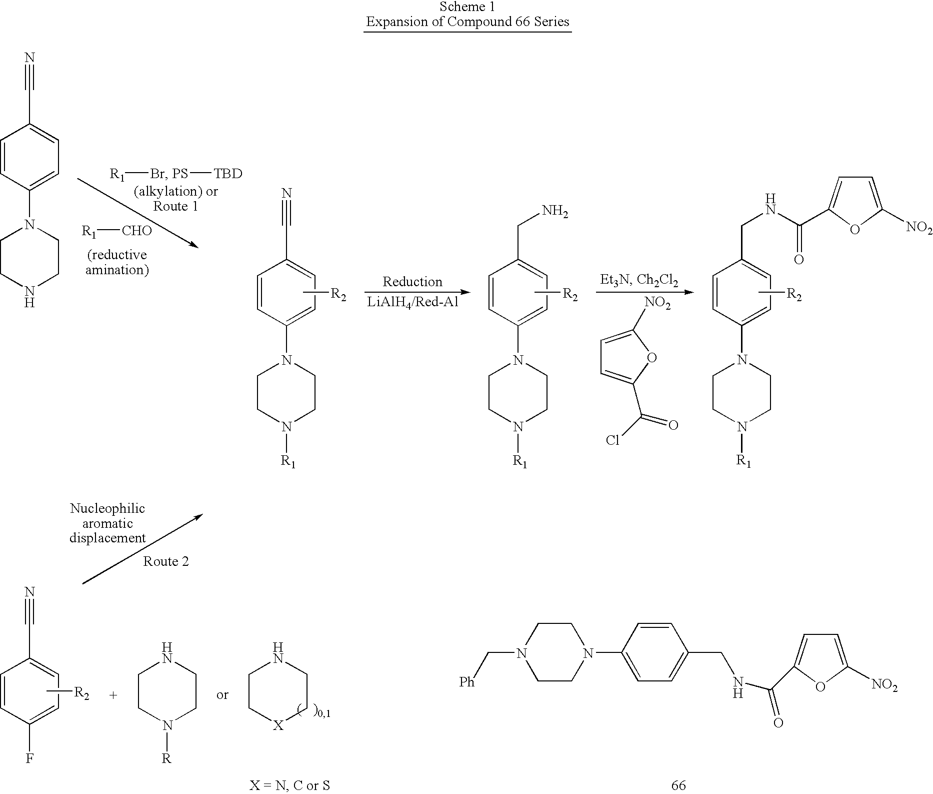

### Scheme 2

Scheme 2 shows synthesis of ether substituted nitrofuranyl benzylamides. To complement the third generation optimization library detailed above the synthesis and activity of substituted phenether nitrofuranyl benzylamides is examined (Scheme 2). The SAR of the 1st generation nitrofuranyl amides (Example 1) indicated that 4-methoxy was the best substitution for bioactivity. N-(4-hydroxybenzyl)-5-nitrofuran-2-carboxamide A was synthesized for use as a starting material to synthesize a sub library of 15 compounds by etherification to the corresponding 2-hydroxylethyl cyclic amines, such as 1-(2-hydroxyethyl)piperidine using Mitsunobu chemistry. See Grese et al., J. Med. Chem. 2001, 44(17), 2857-2860. Some 1-(2-hydroxy ethyl)piperidines are commercially available, and other desirable 2-hydroxylethyl cyclic amines can easily be generated by the reaction of a cyclic secondary amine and ethylene oxide.

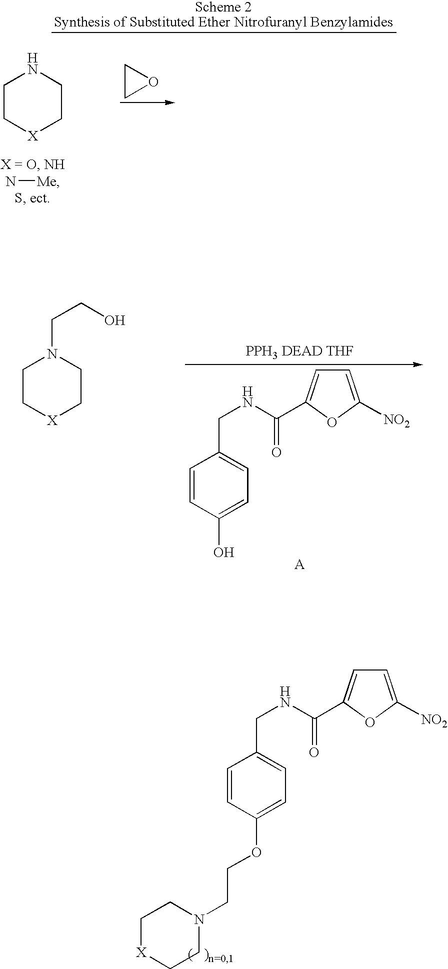

### Scheme 3

Scheme 3 describes synthesis of tertiary nitrofuranylamides with piperazinyl substitutions. The third series to expand is the addition of a piperazine ring to the tertiary amide system of compound 75, to determine if these molecules have enhanced metabolic stabilities over the analogous compounds in the compound 66 series. For the synthesis of this series, 5-fluoroindanone is converted to 6-fluoro-3,4-dihydro-2H-isoquinolin-1-one intermediate, which is the Schmidt rearrangement product, as disclosed in PCT Published Application No. WO 2001057039, incorporated herein in its entirety. The isoquinolinone is subjected to nucleophilic substitution with secondary amines followed by reduction of the amide functionality with BH₃THF to provide the corresponding secondary amine, which can be acylated to give the desired products.

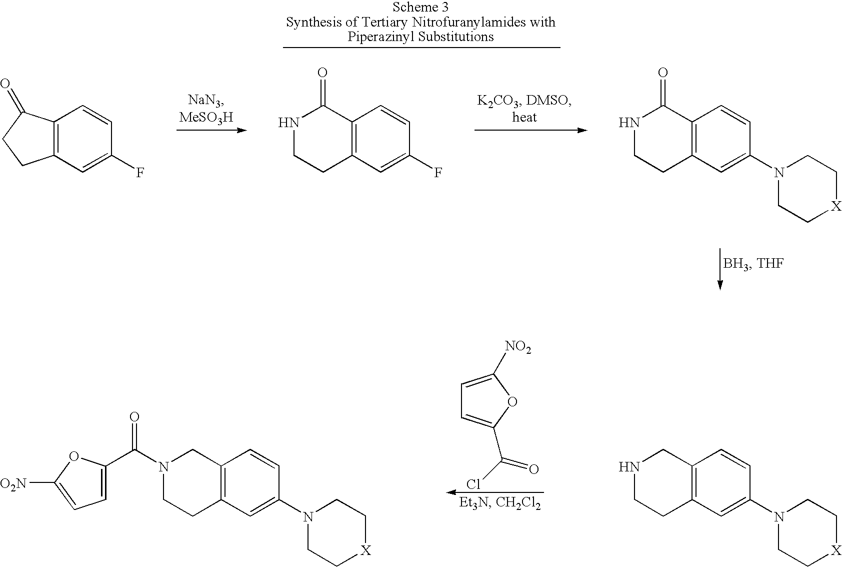

### Examination of Alternative Heterocycles to Nitrofuran

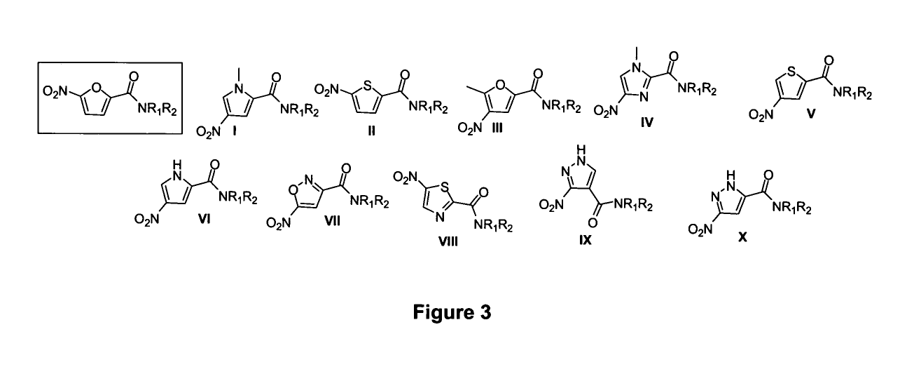

Alternative nitroheterocyclic rings, in addition to nitrofuran, is examined, as an area of SAR optimization. The nitro group plays a role in activity. As such, ten additional nitroheterocylic ring systems for consideration as anti-tuberculosis compounds are shown in FIG. 3.

A representative panel of 10 amides for each nitroheterocycle is synthesized using the same 10 amines in all cases, for example. The amines are selected as representative active amines in the nitrofuran series and the anti-tuberculosis SAR is determined for the heterocyclic ring. Upon observation of activity, an expanded set of amides is synthesized for each heterocycle and evaluated in an optimization program.

Nitroheterocycles thiofuran II, pyrrole VI, thiazole VIII and pyrazote IX and X series can be synthesized from their corresponding commercially available carboxylic acids using standard methods as is generally known in the art. The synthesis of nitropyrrole I and nitroimidazole IV amides is well established for the synthesis of DNA binding polyamides. For example, the Dervan procedure can be used (Baird & Dervan, J. Am. Chem. Soc. 1996, 118, 6141-6146). The thiophene series V can be synthesized by starting from thiophene-2-carboxylic acid, which on nitration gives 5-nitro-thiophene-2-carboxylic acid. See Fu et al., Amer. Chem. Soc. 1999, 121(34), 7761-7759. Further amine coupling affords the desired amides. In order to synthesis series VII, 5-nitro-isoxazole-3-carboxylic acid ethyl ester can be prepared according to the procedure of Diamantini et al. (Giusepie et al., Synthesis, 1993, 11, 1104-1108), and further hydrolysis to the acid followed by amine coupling afford the desired final amides.

### Scheme 4

Referring now to Scheme 4, to complete the SAR of the nitrofuranyl amide series the importance of the amide bond is examined, in that alternative linkages can have improved bioavailablity. Imine analogs are generated by condensing 5-nitro-2-furaldehyde with the same 10 amines selected in the section immediately proceeding. The vinyl analogs are generated using standard Wittig Chemistry.

Additionally, more advanced and constrained cyclic bioisosteric bridges such as benzoxazole, isoxazoline and isoxazole are investigated in evaluating bioavailability. A benzoxazole bridge is formed by condensation of the amide to C-2 aromatic hydroxyl of 2-aminophenol. Isoxazolines and isoxazoles are synthesized from aryl aldehydes, which are converted to oximes reaction with by simple hydroxylamine hydrochloride. See Barbachyn et al., J. Med. Chem. 2003, 46(2), 284-302; and Choi et al., J. Bacteriol. 2001, 183, 7058-7066. A nitrile oxide generated from corresponding oximes on reaction with olefins gives isoxazolines. Similarly, isoxazoles can be prepared by reacting alkynes instead of olefins with nitrile oxide. The orientation of furan and aryl groups over the oxazole/oxazoline ring can be directed by exchanging oxime-yne/ene functionality between those two groups.

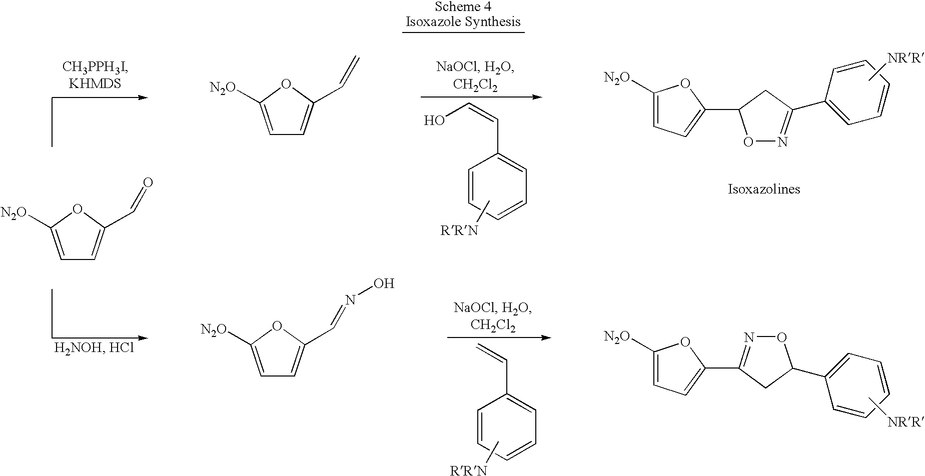

### Example 4

### Scheme 1

Referring now to Scheme 1, the compounds 92 and 96 were synthesized starting from p-fluorobenzonitrile. The fluoro group was substituted with the corresponding cyclic secondary amines. Then the nitrile functionality was reduced with red-Al in case of thiomorpholine substituted benzonitrile and with rany-Ni hydrogenation in case of N-Boc piperazine substituted benzonitrile to give the corresponding primary amines, which were then treated with 5-nitro-furan-2-carbonyl chloride to give the final product compounds 92 and 96. The synthesis of compound 98 was carried out in similar way to compound 92 starting from 4-(4-cyclopropylmethyl-piperazin-1-yl)-benzonitrile.

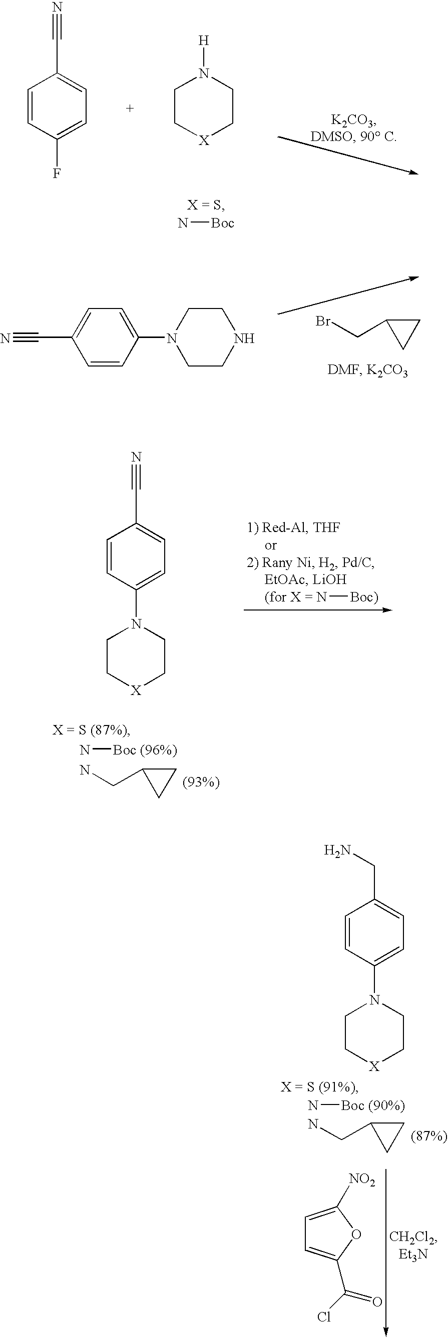

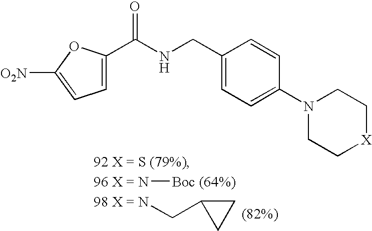

### Scheme 2

Referring now to Scheme 2, substitution of fluorine on 4-fluorobenzonitrile with (S)-(−)-3-(Boc-amino) pyrrolidine followed by reduction of nitrile group with red-Al gave the corresponding benzylamine. The benzylamine was treated with 5-nitro-furan-2-carbonyl chloride to give the intermediate with on Boc-deprotection gave the product compound 95.

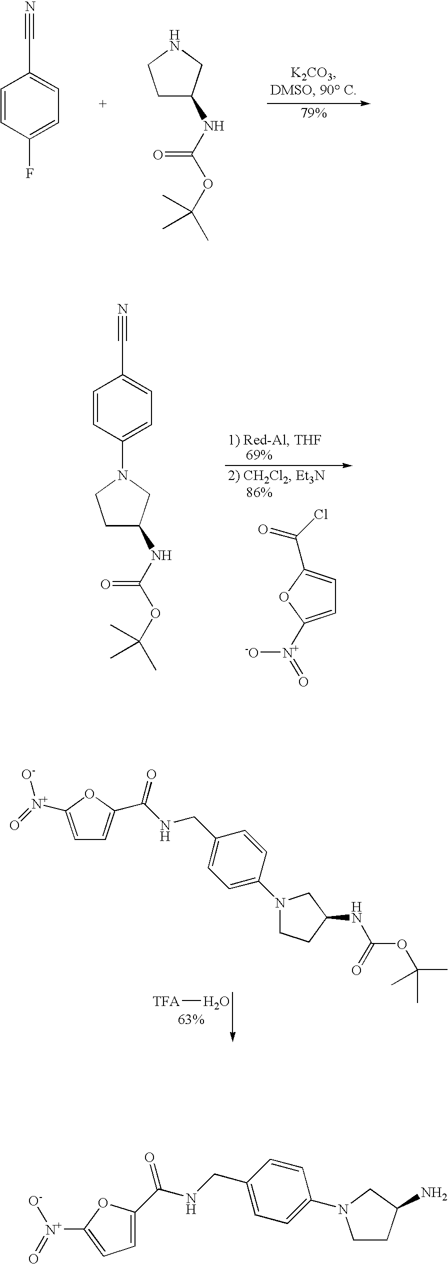

### Scheme 3

Referring now to Scheme 3, compound 92 was further treated with m-chloroperbenzoic acid to give oxidized product compounds 93 and 94.

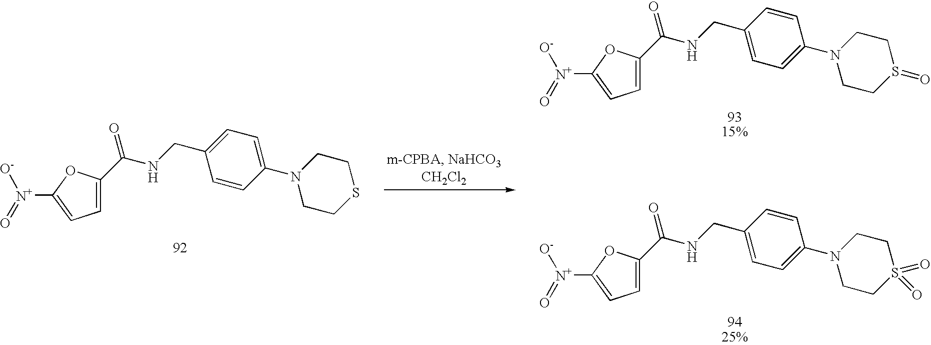

### Scheme 4

Referring now to Scheme 4, the boc-protecting group on compound 96 was removed by treating with trifluoroacetic acid in water to give free amine compound 97.

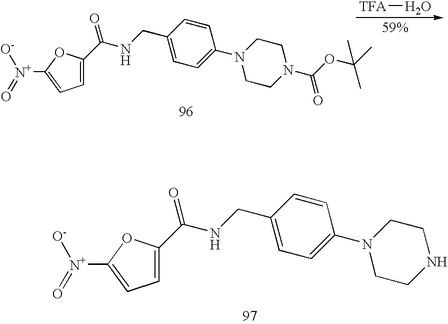

### Scheme 5

Referring now to Scheme 5, the targeted products were synthesized starting from 3,4-difluorobenzonitrile, which on reaction with cyclic secondary amides in presence of base substituted the para-fluorine group and gave the corresponding tertiary amines. Subsequently the nitrile group was reduced with red-Al to give corresponding benzylamines, which were then treated with 5-nitro-furan-2-carbonyl chloride to give the final targeted product compounds 99-103.

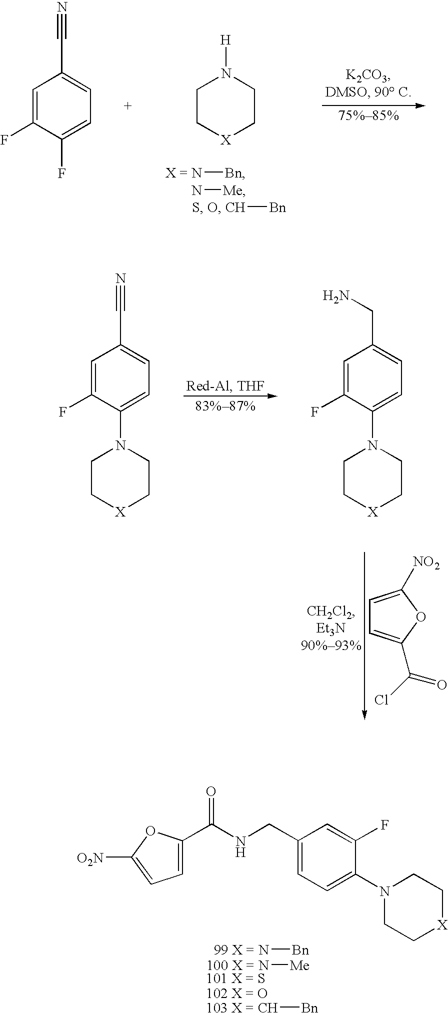

### Scheme 6

Referring now to Scheme 6, compound 97 was treated with ethylchloroformate in presence of triethylamine to give the carbamate compound 104. Similarly, compound 97 on reaction with n-propyl isocyanate and isopropyl isocyanate gave the urea derivatives compounds 105 and 106, respectively.

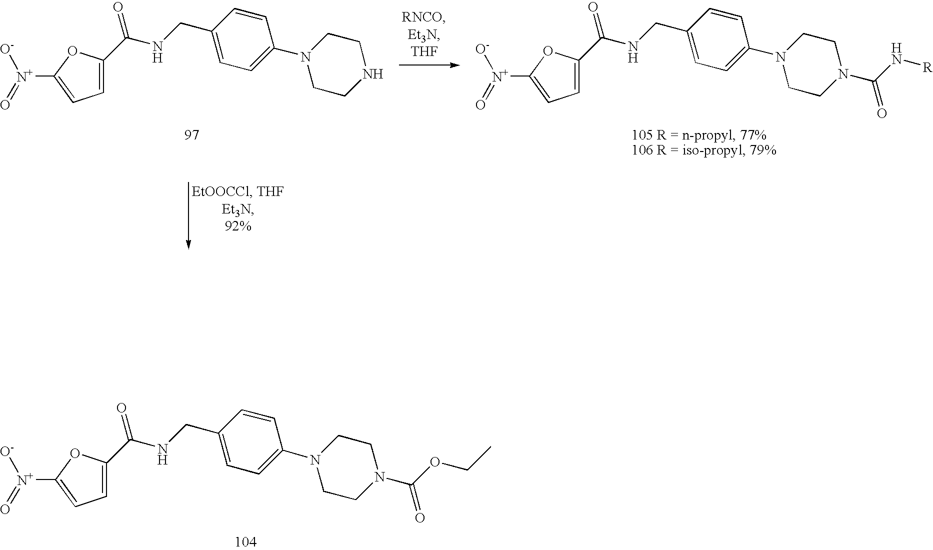

### Biological Results

The MIC of the nitrofuranyl amides against M tuberculosis H37Ra was determined by the micro broth dilution method using microtiter plates. M. tuberculosis was grown in Middlebrook 7H9 medium to an OD₆₅₀ of 0.4-0.6 and a dilution made to an. OD₆₅₀of 0.01. 100 μl of these cells are then added to a microtiter well containing serial dilutions of the nitrofuranyl amides. The cells are then incubated at 37° C. for 7 days and visually examined for growth. MIC₉₀was determined for wells with greater than 90% inhibition of growth. Results are shown in Table 5 below.

TABLE 5

| No. | Compound | MIC (μg/ml) |
|---|---|---|
| 92 | 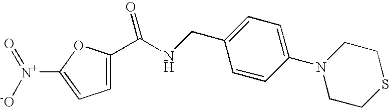 | 0.05 |
| | 5-Nitro-furan-2-carboxylic acid 4-thiomorpholin-4-yl-benzylamide | |
| 93 | 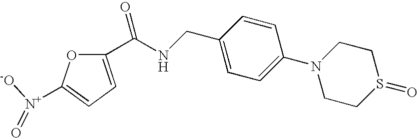 | 0.8 |
| | 5-Nitro-furan-2-carboxylic acid 4-(1-oxo-¼-thiomorpholin-4-yl)-benzylamide | |
| 94 | 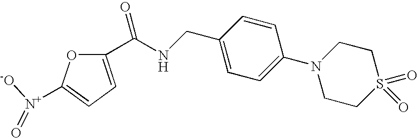 | 6.25 |
| | 5-Nitro-furan-2-carboxylic acid 4-(1,1-dioxo-⅙-thiomorpholin-4-yl)-benzylamide | |
| 95 | 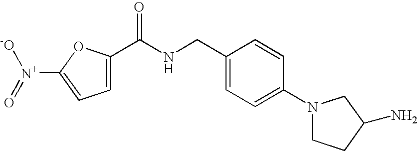 | 0.8 |
| | 5-Nitro-furan-2-carboxylic acid 4-(3-amino-pyrrolidin-1-yl)-benzylamide | |
| 96 | 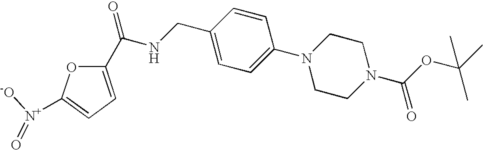 | 0.0002 |
| | 4-(4-{[(5-Nitro-furan-2-carbonyl)-amino]-methyl}- phenyl)-piperazine-1-carboxylic acid tert-butyl ester | |
| 97 | 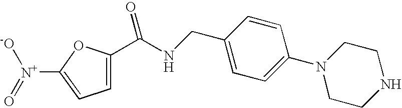 | 0.8 |
| | 5-Nitro-furan-2-carboxylic acid 4-piperazin-1-yl-benzylamide | |
| 98 | 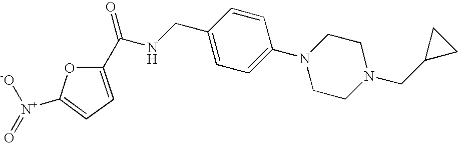 | 0.05 |
| | 5-Nitro-furan-2-carboxylic acid 4-(4-cyclopropylmethyl- piperazin-1-yl)-benzylamide | |
| 99 | 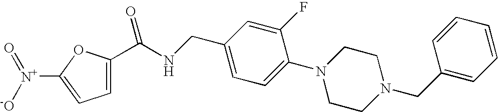 | 0.0125 |
| | 5-Nitro-furan-2-carboxylic acid 4-(4-benzyl-piperazin-1- yl)-3-fluoro-benzylamide | |
| 100 | 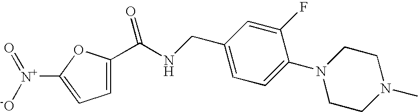 | 0.4 |
| | 5-Nitro-furan-2-carboxylic acid 3-fluoro-4- (4-methyl-piperazin-1-yl)-benzylamide | |
| 101 | 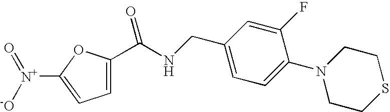 | 0.2 |
| | 5-Nitro-furan-2-carboxylic acid 3-fluoro-4- thiomorpholin-4-yl-benzylamide | |
| 102 | 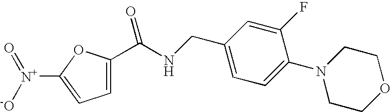 | 0.2 |
| | 5-Nitro-furan-2-carboxylic acid 3-fluoro-4-morpholin-4-yl-benzylamide | |
| 103 | 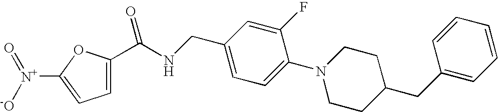 | 0.0125 |
| | 5-Nitro-furan-2-carboxylic acid 4-(4-benzyl-piperidin- 1-yl)-3-fluoro-benzylamide | |
| 104 | 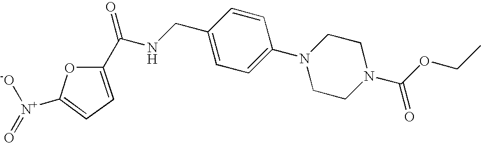 | 0.004 |
| | 4-(4-{[(5-Nitro-furan-2-carbonyl)-amino]-methyl}-phenyl)- piperazine-1-carboxylic acid ethyl ester | |
| 105 | 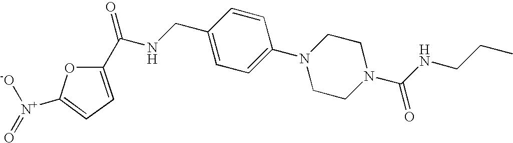 | 0.0125 |
| | 4-(4-{[(5-Nitro-furan-2-carbonyl)-amino]-methyl}-phenyl)- piperazine-1-carboxylic acid propylamide | |
| 106 | 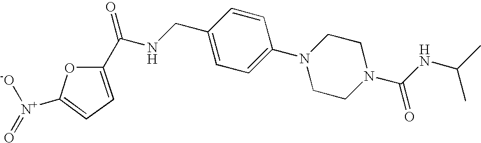 | 0.0062 |
| | 4-(4-{[(5-Nitro-furan-2-carbonyl)-amino]-methyl}-phenyl)- piperazine-1-carboxylic acid isopropylamide | |

### Example 5

### Methods and Materials

All the anhydrous solvents and starting materials were purchased from Aldrich Chemical Company (Milwaukee, Wis., U.S.A.). All reagent grade solvents used for chromatography were purchased from Fisher Scientific (Suwanee, Ga., U.S.A.) and FLASH™ column chromatography silica cartridges were obtained from Biotage Inc. (Lake Forest, Va., U.S.A.). The reactions were monitored by thin layer chromatography (TLC) on pre-coated Merck 60 F₂₅₄ silica gel plates and visualized using UV light (254 nm). Biotage FLASH 25+™ column chromatography system was used to purify mixtures. All ¹H and ¹³C NMR spectra were recorded on a Bruker ARX-300 (300 and 75 MHz for ¹H and ¹³C NMR, respectively; Billerica, Mass., U.S.A.) or Varian INOVA-500™ (500 and 125 MHz for ¹H and ¹³C NMR, respectively; Palo Alto, Calif., U.S.A.) spectrometer. Chemical shifts are reported in ppm (δ) relative to residual solvent peak or internal standard (tetramethylsilane) and coupling constants (J) are reported in hertz (Hz) (s=singlet, bs=broad singlet, d=doublet, dd=double doublet, bd=broad doublet, ddd=double doublet of doublet, t=triplet, tt=triple triplet, q=quartet, hep=heptet, m=multiplet). Mass spectra were recorded on a Bruker ESQUIRE LCMS™ using ESI. Purity of the final products was confirmed before testing by analytical HPLC using a Alltech (Deerfield, Ill., U.S.A.) platinum C-18 reverse phase column (4.5×150mm) and a H₂O (0.1% TFA) to acetonitrile 0-100% linear gradient at a flow rate of 1.0 mL min⁻¹ and UV detection at 254 nm. The yields quoted are unoptimized.

### Scheme 1

Referring now to Scheme 1, to evaluate the antituberculosis activity and SAR of nitrofuranyl substituted piperazines 5-nitrofuranyl piperazine and piperidine amides with various substituents, compounds 72, 73, 76-82, 91, 107, and 108 were synthesized by reacting the corresponding amines with 5-nitro 2-furan carbonyl chloride, the later was obtained from 5-nitro 2-furan carboxylic acid 5a (Scheme 1).

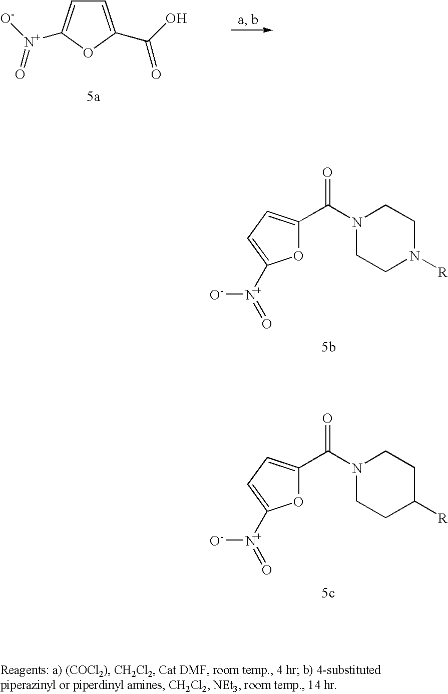

### Scheme 2

Referring now to Scheme 2, to evaluate the importance of the substituents on the benzyl ring, synthesis was performed as delineated below in Scheme 2. 5-Nitrofuran 2-carboxylic acid 5a was converted to 5-nitro furan 2-carbonyl chloride which upon reacting with Boc piperazine afforded compound 10a, subsequent deprotection of Boc was achieved using TFA: H₂O (1:1) to give compound 10b. Reductive amination of substituted aldehydes (see Abdel-Magid et al., J. Org. Chem. 1996, vol. 61, pp. 3849-3862) with free amine 10b afforded the desired products 83-90 (Scheme-2).

Further investigation lead to the replacement of the benzylic amines with tertiary amides. This was achieved by transformation of the respective benzoic acids to benzoyl chlorides and subsequently reacting with (5-nitro-furan-2-yl)-piperazin-1-yl-methanone 10b provided corresponding diamides 109 and 110.

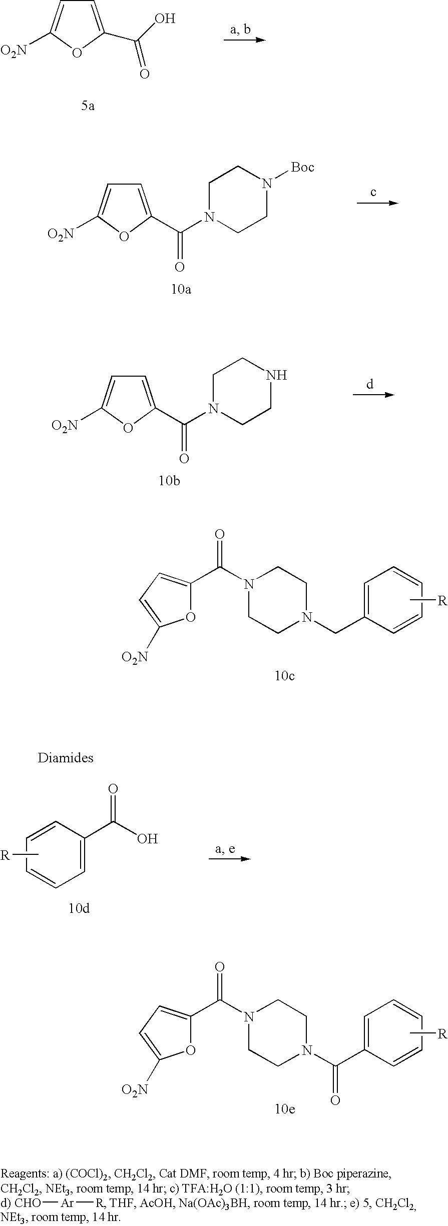

### Scheme 3

Referring now to Scheme 3, in order to study the pharmacokinetic properties of the compounds, hydrazone was introduced. This was achieved by the reduction of the cyano group using lithium aluminum hydride (see Lee et al., J. Med. Chem. 2003, vol. 46(14), pp. 3116-3126) to give corresponding amine 11b. Which upon further reacting with 5-nitro-furan-2-carbonyl chloride produced compound 11c. The hydroxyl group on 11c was subsequently oxidized to a formyl group using Dess-Martin periodinane (1,1,1-triacetoxy-1,1-dihydro-1,2-benziodoxol-3(1H)-one; hereinafter, “DMP”). See Dess et al., J. Org. Chem. 1983, vol. 48, pp. 4155-4156 and Dess et al., J. Am. Chem. Soc. 1991, vol. 113, pp. 7277-7287. For improved procedures for the preparation of DMP, see Ireland et al., J. Org. Chem. 1993, vol. 58, p. 2899 and Meyer et al., J. Org. Chem. 1994, vol. 59, pp. 7549-7552. This was derivatized to the hydrazone compound 111. See Domagala et al., J. Med. Chem. 1986, pp. 394-404.

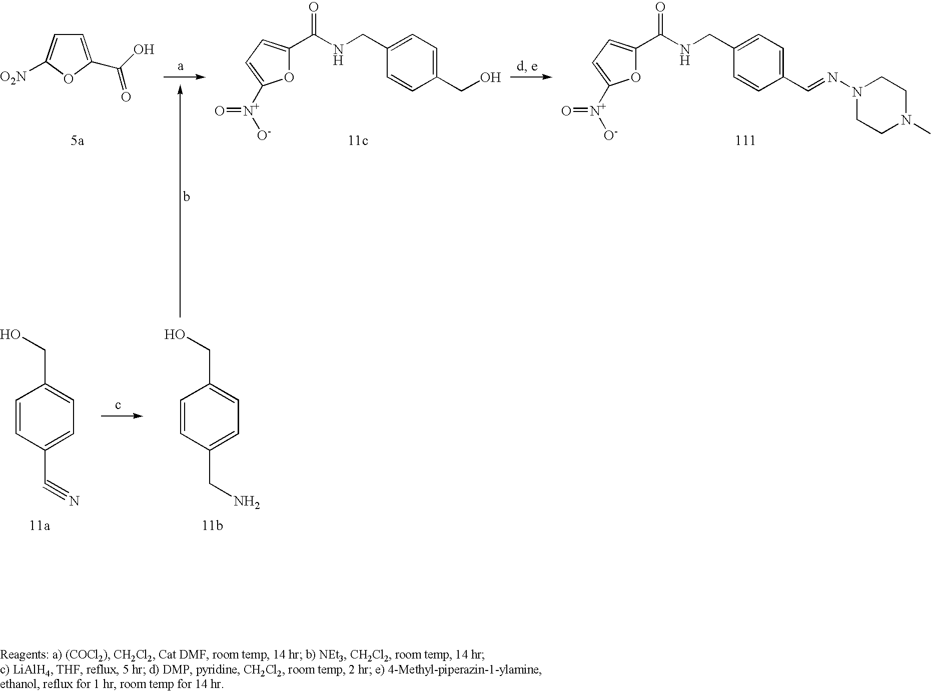

Preparation of 5-Nitro-furan-2-carbonyl chloride. 5-Nitro-furan-2-carboxylic acid (3 g, 19.1 mmol) in CH₂Cl₂ (25 mL) was treated with oxalylchloride (3.332 mL, 38.2 mmol) followed by 2 drops of DMF and stirred at room temperature for 4 hrs. The reaction mixture was concentrated in vacuum to obtain acid chloride and the crude was used in further reactions without purification.

General procedure for preparation of amides 72, 73, 76-82, 91, 107 and 108. 5-Nitro-furan-2-carbonyl chloride (930 mg, 5.3 mmol) in CH₂Cl₂ (10 mL) was added to amine (3.18 mmol, 1 equiv) in Et₃N (3 mL) and the mixture was stirred for 14 hrs at room temperature. Reaction was followed by TLC, after completion of reaction 100 mL of ethyl acetate was added and washed with 10% aqueous NaHCO₃ (2×50 mL), water (2×50 mL) and brine (2×50 mL). The organic phase was dried (Na₂SO₄), filtered and concentrated followed by flash column purification and recrystallization provided corresponding amides.

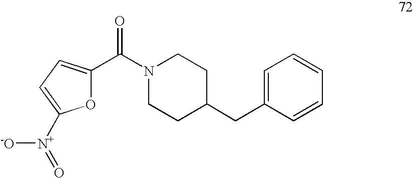

(4-Benzyl-piperidin-1yl)-5-nitro-furan-2-yl)-methanone (72). 5-Nitro-furan-2-carbonyl chloride (930 mg, 5.3 mmol) in CH₂Cl₂ (10 mL) was added to 4-benzyl piperidine (931 μL, 5.3 mmol) in Et₃N (3 mL). The reaction mixture was stirred for 14 hrs at room temperature. The reaction was followed as explained above to yield 1.24 g (74%) of compound 72; TLC: R_{f}0.55 (1:1 petroleum-ether: ethyl acetate); ¹HNMR (500 MHz, CDCl₃): δ 1.22-1.41 (2H, bs), 1.78-1.85 (2H, 2bs), 1.85-1.94 (1H, m), 2.61 (2H, d, J=6.84 Hz), 2.77 (1H, t, J=11.32 Hz); 3.17(1H, t, J=11.32 Hz),4.32(1H, d, J=11.71 Hz), 4.62(1H, d, J=11.23 Hz), 7.12(1H, d, J=3.66 Hz), 7.17(2H, d, J=7.07 Hz), 7.23(1H, t, J=7.32 Hz), 7.31(2H, t, J=7.56 Hz), 7.35(1H, d, J=3.91 Hz); ¹³CNMR (300 MHz, CD₃OD:CDCl₃=1:3): 31.13, 32.16, 37.59, 42.21, 43.06, 46.43, 111.16, 116.68, 125.60, 127.81, 128.52, 139.15, 148.55, 156.19; ESI-Mass: 337 (M+23); Anal. Calcd. For C₁₃H₁₀N₂O₄: C, 64.96; H, 5.77; N, 8.91. Found: C, 64.7; H, 5.76; N, 8.92.

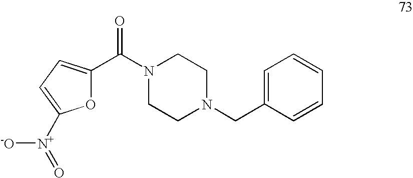

(4-Benzyl-piperazin-1yl)-5-nitro-furan-2-yl)-methanone (73). 5-Nitro-furan-2-carbonyl chloride (930 mg, 5.3 mmol) in CH₂Cl₂ (10 mL) was added to 1-benzyl piperazine (921 μL, 5.3 mmol) in Et₃N (3 mL). The reaction mixture was stirred for 14 hrs at room temperature. The reaction was followed as explained above to yield 910 mg (55%) of compound 73; TLC: R_{f}0.23 (1:1 petroleum ether: ethyl acetate); ¹HNMR (500 MHz, CDCl₃): δ 3.53 (2H, s), 3.59-3.78 (4H, 2bs), 7.24-7.29 (2H, m), 7.31-7.3 (4H, m), 7.76 (1H, d, J=3.66 Hz);

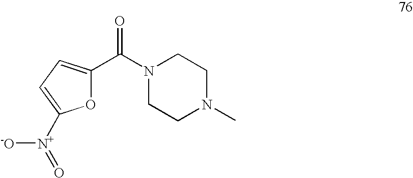

4-(Methyl-piperazin-1yl)-5-nitro-furan-2-yl)-methanone (76). 5-Nitro-furan-2-carbonyl chloride (877mg, 5mmol) in CH₂Cl₂ (10 mL) was added to 1-methyl piperazine (553 μL, 5mmol) in Et₃N (3 mL). The reaction mixture was stirred for 14hrs at room temperature. The reaction was followed as explained above to yield 600 mg (50%) of compound 76; R_{f} 0.46(1:9 methanol: chloroform); ¹HNMR(500 MHz, CDCl₃): δ 2.36 (3H, s), 2.52 (4H, t, J=5.13 Hz), 3.72-4.00 (4H, 2bs), 7.18 (1H, d, J=3.91 Hz), 7.36 (1H, d, J=3.91 Hz); ¹³CNMR (300 MHz, CDCl₃): 42.58, 45.36, 45.96, 54.03, 54.68, 111.07, 117.37, 148.19, 156.09; ESI-Mass: 262.2 (M+23); Anal. Calcd for C₁₀H₁₃N₃O₄: C, 50.21; H, 5.48; N, 17.56. Found: C, 49.91; H, 5.36; N, 17.39.

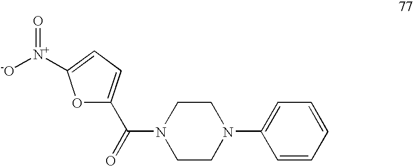

(5-Nitro-furan-2-yl)-(4-phenyl-piperazin-1-yl)-methanone (77). 5-Nitro-furan-2-carbonyl chloride (558 mg, 3.18 mmol) in CH₂Cl₂ (10 mL) was added to 1-phenyl-piperazine (485 μL, 3.18 mmol) in Et₃N (3 mL). The reaction mixture was stirred for 14 hrs at room temperature. The reaction was followed as explained above to yield 540 mg (56%) of compound 77; TLC: R_{f} 0.71 (ethyl acetate); ¹HNMR (500 MHz, CDCl₃): δ 3.31 (4H, t, J=5.13 Hz), 3.88-4.18 (4H, 2bs), 6.96 (3H, dd, J=7.32, 14.64 Hz), 7.24(1H, d, J=3.66 Hz), 7.32(2H, dd, J=7.33, 8.79 Hz), 7.38(1H, d, J=3.91 Hz); ¹³CNMR (300 MHz, CDCl₃): 42.01, 46.06, 48.32, 112.79, 115.8, 117.08, 119.36, 128.94, 147.54, 150.47, 156.59; ESI-Mass: 324.2 (M+23); Anal. Calcd for C₁₅H₁₅N₃O₄: C, 59.79; H, 5.02; N, 13.97. Found: C, 59.55; H, 4.91; N, 13.74.

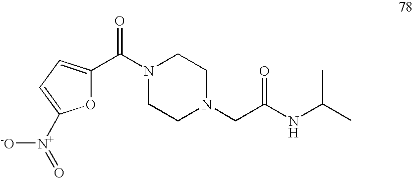

N -Isopropyl-2-[4-(5-nitro-furan-2-carbonyl)-piperazin-1-yl]-acetamide (78). 5-Nitro-furan-2-carbonyl chloride (558 mg, 3.18 mmol) in CH₂Cl₂ (10 mL) was added to N-isopropyl-1-piperazine acetamide (588 mg, 3.18 mmol) in Et₃N (3 mL). The reaction mixture was stirred for 14 hrs at room temperature. The reaction was followed as explained above to yield 520 mg (50%) of compound 78; TLC: R_{f} 0.10(ethyl acetate); ¹HNMR (500 MHz, CDCl₃): 61.18 (3H, s), 1.2 (3H, s), 2.63 (1H, t, J=5.13 Hz), 3.07(2H, s), 3.76-3.79 (4H, m), 4.04 (1H, hep, J=6.59 Hz), 7.21 (1H, d, J=3.9 Hz), 7.53 (1H, d, J=3.91 Hz); ¹³CNMR (300 MHz, DMSO): 22.69, 42.10, 46.14, 52.59, 60.62, 112.76, 116.84, 147.58, 151.1, 156.57, 167.68; ESI-Mass: 324.33 (M+23); Anal. Calcd for C₁₄H₂₀N₄O₅.

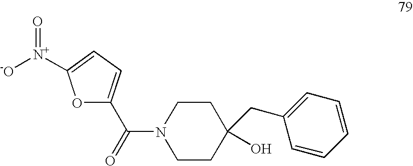

(4-Benzyl-4-hydroxy-piperidin-1-yl)-(5-nitro-furan-2-yl)-methanone (79). 5-Nitro-furan-2-carbonyl chloride (558 mg, 3.18 mmol) in CH₂Cl₂ (10 mL) was added to 4-benzyl-4-hydroxy piperidine (607 mg, 3.18 mmol) in Et₃N (3 mL). The reaction mixture was stirred for 14 hrs at room temperature. The reaction was followed as explained above to yield 480 mg (45%) of compound 79; TLC: R_{f} 0.60 (ethyl acetate); ¹HNMR (500 MHz, DMSO): δ 1.4-1.6 (4H, m), 2.72 (2H, s), 3.05-3.2 (1H, bs), 3.46(1H, t, J=11.72 Hz), 3.87 (1H, d, J=11.71 Hz), 4.14 (1H, d, J=11.47 Hz), 4.58 (1H, s), 7.17-7.24 (4H, m), 7.74 (1H, d, J=3.91 Hz); ¹³CNMR (300 MHz, DMSO): 35.7, 36.69, 42.57, 48.27, 68.36, 112.77, 116.33, 125.84, 127.54, 130.5, 137.31, 148,151, 156.44; ESI-Mass: 353.1 (M+23); Anal. Calcd for C₁₇H₁₈N₂O₅: C, 61.81; H, 5.49; N, 8.48. Found: C, 61.68; H, 5.43; N, 8.45.

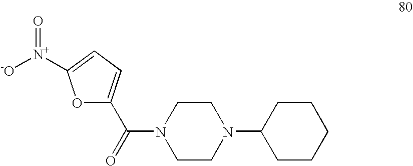

(4-Cyclohexyl-piperazin-1-yl)-(5-nitro-furan-2-yl)-methanone (80). 5-Nitro-furan-2-carbonyl chloride (558 mg, 3.18 mmol) in CH₂Cl₂ (10 mL) was added to 1-Cyclohexyl-piperazine (534 mg, 3.18 mmol) in Et₃N (3 mL). The reaction mixture was stirred for 14 hrs at room temperature. The reaction was followed as explained above to yield 610 mg (62%) of compound 80; TLC: R_{f} 0.21 (ethyl acetate); ¹HNMR (500 MHz, DMSO): 6 1-1.2 (1H, m), 1.12-1.28 (4H, m), 1.57 (1H, d, J=11.96 Hz), 1.68-1.82 (4H, m), 2.24-2.34 (1H, m), 2.53 (4H, t, J=4.88 Hz), 2.54 (4H, t, J=4.88 Hz), 7.25 (1H, d, J=3.66 Hz), 7.76 (1H, d, J=3.9 Hz); ¹³CNMR (300 MHz, DMSO): 25.19, 25.76, 28.2, 42.72, 46.81, 48.14, 48.8, 62.49, 112.75, 116.71, 147.71, 151.06, 156.4; ESI-Mass: 330.1 (M+23); Anal. Calcd for C₁₅H₂₁N₃O₄: C, 58.62; H, 6.89; N, 13.67. Found: C, 58.67; H, 7.0; N, 13.64.

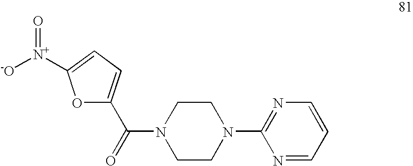

(5-Nitro-furan-2-yl)-(4-pyrimidin-2-yl-piperazin-1-yl)-methanone (81). 5-Nitro-furan-2-carbonyl chloride (558 mg, 3.18 mmol) in CH₂Cl₂ (10 mL) was added to 2-(1-piperazinyl)-pyrimidine 2HCl (753 mg, 3.18 mmol) in Et₃N (3 mL). The reaction mixture was stirred for 14 hrs at room temperature. The reaction was followed as explained above to yield 480 mg (49%) of compound 81; TLC: R_{f} 0.60 (ethyl acetate); ¹HNMR (500 MHz, DMSO): 6 3.6-4 (8H, m), 6.69 (1H, t, J=4.63 Hz), 7.32 (1H, d, J=3.9 Hz), 7.79 (1H, d, J=3.66 Hz), 8.41 (2H, d, J=4.63 Hz); ¹³CNMR (300 MHz, DMSO): 43.1, 45.96, 110.53, 112.78, 116.97, 147.48, 156.88, 157.94, 160.96; ESI-Mass: 326.3 (M+23); Anal. Calcd for C₁₅H₂₁N₃O₄: C, 51.48; H, 4.32; N, 23.09. Found: C, 51.62; H, 4.57; N, 22.87.

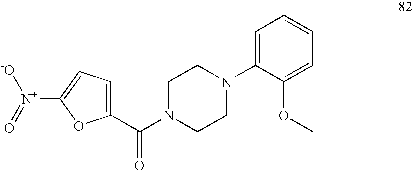

[4-(2-Methoxy-phenyl)-piperazin-1-yl]-(5-nitro-furan-2-yl)-methanone (82). 5-Nitro-furan-2-carbonyl chloride (558 mg, 3.18 mmol) in CH₂Cl₂ (10 mL) was added to 1-(2-methoxy phenyl) piperazine (558 μL, 3.18 mmol) in Et₃N (3 mL). The reaction mixture was stirred for 14 hrs at room temperature. The reaction was followed as explained above to yield 780 mg (73%) of compound 82; TLC: R_{f} 0.69 (ethyl acetate); ¹HNMR (500 MHz, CD₃OD): δ 3.17 (4H, t, J=5.12 Hz), 3.93 (3H, s), 3.94-4.1 (4H, 2bs), 6.96 (1H, dt, J=1.7, 8.05 Hz), 7.03 (2H, ddd, J=1.46, 5.37, 8.1 Hz), 7.09 (1H, dt, J=1.7, 7.07 Hz), 7.28 (1H, d, J=3.9 Hz), 7.59 (1H, d, J=3.66 Hz); ¹³CNMR (300 MHz, DMSO): 42.43, 46.58, 50.3, 55.3, 111.87, 112.77, 116.96, 118.31, 120.76, 122.95, 140.43, 147.58, 151.15, 151.95, 156.58; ESI-Mass: 332 (M+1); Anal. Calcd for C₁₆H₁₇N₃O₅: C, 58.0; H, 5.17; N, 12.68. Found: C, 57.84; H, 5.12; N, 12.7.

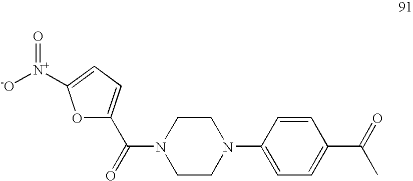

1-{4-[4-(5-Nitro-furan-2-carbonyl)-piperazin-1-yl]-phenyl}-ethanone (91). 5-Nitro-furan-2-carbonyl chloride (558 mg, 3.18 mmol) in CH₂Cl₂ (10 mL) was added to 4-piperazino acetophenone (649 mg, 3.18 mmol) in Et₃N (3 mL). The reaction mixture was stirred for 14 hrs at room temperature. The reaction was followed as explained above to yield 540 mg (55%) of compound 91; TLC: R_{f} 0.68 (ethyl acetate); ¹HNMR (500 MHz, DMSO) δ 2.24 (3H, s), 3.49 (4H, t, J=4.88 Hz), 3.72-3.96 (4H, 2bs), 7.0 (2H, d, J=9.03 Hz), 7.34 (1H, d, J=3.9 Hz), 7.8 (1H, d, J=3.9 Hz), 7.84 (2H, d, J=8.78 Hz); ¹³CNMR (300 MHz, DMSO): 26.0, 41.84, 46.3, 112.78, 113.12, 117.22, 126.91, 130.02, 147.47, 151.18, 153.16, 156.69; ESI-Mass: 344 (M+1); Anal. Calcd for C₁₇H₁₇N₃O₅: C, 59.47; H, 4.99; N, 12.24. Found: C, 59.19; H, 5.08; N, 11.98.

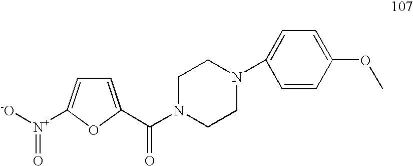

[4-(4-Methoxy-phenyl)-piperazin-1-yl]-(5-nitro-furan-2-yl)-methanone (107). 5-Nitro-furan-2-carbonyl chloride (558 mg, 3.18 mmol) in CH₂Cl₂ (10 mL) was added to 1-(4-Methoxy-phenyl)-piperazine (611 mg, 3.18 mmol) in Et₃N (3 mL). The reaction mixture was stirred for 14 hrs at room temperature. The reaction was followed as explained above to yield 910 mg (86%) of compound 107; TLC: R_{f} 0.4 (1:1 petroleum ether: ethyl acetate); ¹HNMR (500 MHz, DMSO) δ 3.08 (4H, t, J=5.12 Hz), 3.69 (3H, s), 3.72-3.92 (4H, 2bs), 6.84 (2H, d, J=9.03 Hz), 6.94 (1H, d, J=9.03 Hz), 7.32 (1H, d, J=3.9 Hz), 7.79 (1H, d, J=39 Hz); ¹³CNMR (300 MHz, DMSO): 42.27, 46.27, 49.79, 55.12, 112.77, 114.26, 117.04, 118.02, 118.31, 144.82, 147.56, 151.16, 153.37, 156.55; ESI-Mass: 332.3 (M+1); Anal. Calcd for C₁₆H₁₇N₃O₅: C, 58.00; H, 5.17; N, 12.68. Found: C, 58.03; H, 5.16; N, 12.6.

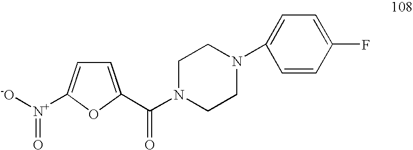

[4-(4-Fluoro-phenyl)-piperazin-1-yl]-(⁵-nitro-furan-2-yl)-methanone (108). 5-Nitro-furan-2-carbonyl chloride (558 mg, 3.18 mmol) in CH₂Cl₂ (10 mL) was added to 1-(4-Fluoro-phenyl)-piperazine (573 mg, 3.18 mmol) in Et₃N (3 mL). The reaction mixture was stirred for 14 hrs at room temperature. The reaction was followed as explained above to yield 850 mg (83%) of compound 108; TLC: R_{f} 0.5 (1:1 petroleum ether:ethyl acetate); ¹HNMR (500 MHz, DMSO) δ 3.16 (4H, t, J=4.88 Hz), 3.72-3.92 (4H, 2bs), 6.99 (2H, dd, J=4.88, 9.27 Hz), 7.08(2H, t, J=8.54 Hz), 7.32 (1H, d, J=3.9 Hz), 7.78 (1H, d, J=3.9 Hz); ¹³CNMR (300 MHz, DMSO): 42.13, 46.07, 49.14, 112.78, 115.16, 115.45, 117.09, 117.67, 117.77, 147.4, 147.52, 154.74, 156.58, 157.87; ESI-Mass: 320.3 (M+1); Anal. Calcd for C₁₅H₁₄N₃O₄: C, 56.43; H, 4.42; N, 13.16. Found: C, 56.31; H, 4.41; N, 13.06.

General procedure for preparation of amides 413-419 and 433.

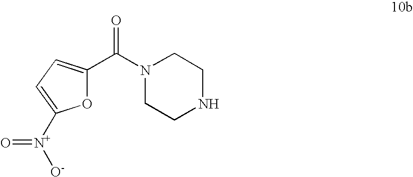

4-(5-Nitro-furan-2-carbonyl)-piperazine-1-carboxylic acid tert-butyl ester (10b). 5-Nitro-furan-2-carbonyl chloride (3.346 g, 19.1 mmol) in CH₂Cl₂ (40 mL) was added to boc-piperazine (3.556 g, 19.1 mmol) in Et₃N (5 mL) and the mixture was stirred for 14 hrs at room temp. Reaction was followed by TLC, after completion of the reaction 2×200 mL of ethyl acetate was added and washed with 10% aqueous NaHCO₃ (2×50 mL), water (2×50 mL) and brine (2×50 mL). The organic phase was dried over Na₂SO₄, filtered and concentrated to yield 5.31 gm (85%) of compound 10b. ¹HNMR (500 MHz, CD₃OD) δ 1.49 (9H, s), 3.55 (4H, s), 3.64-3.91 (4H, 2bs), 7.23 (1H, d, J=3.90 Hz), 7.53 (1H, d, J=3.90 Hz); ESI-Mass: 348.6 (M+23).

(5-Nitro-furan -2yl)-piperazin-1-yl-methanone. 4-(5-Nitro-furan-2-carbonyl)-piperazine-1-carboxylic acid tert-butyl ester 10b in 40 mL of TFA: H₂O (1:1) was stirred for 3hrs at room temp. and the reaction was followed by TLC, after completion of the reaction 3×100 mL of chloroform was added and washed with Na₂CO₃ (2×50 mL), water (2×50 mL) and brine (2×50 mL). The organic phase was dried over Na₂SO₄, filtered (5-Nitro-furan-2-yl)-piperazin-1-yl-methanone (Yield, 3.04 g, 82%) which was carried out for further reactions with out purification; ¹HNMR (500 MHz, CD₃OD) δ 2.92 (4H, t, J=4.88 Hz), 3.61-3.92 (4H, 2bs), 7.21 (1H, d, J=3.90 Hz), 7.53 (1H, d, J=3.66 Hz); ESI-Mass: 325.9 (M+1).

General Procedure. (5-Nitro-furan-2-yl)-piperazin-1-yl-methanone (400 mg, 1.77 mmol) in THF (20 mL) was added to substituted benzaldehyde (1.77 mmol) followed by AcOH (2 mL) and stirred for 10 minutes at room temperature. Then Na(OAC)₃BH (525 mg, 2.48mmol) was added and the reaction mixture was stirred for 14 hrs at room temperature. The reaction was followed by TLC and the reaction mixture was neutralized using NaHCO₃ (2×50 mL), followed by 200 mL of ethyl acetate and brine (2×50 mL). The organic phase was dried (Na₂SO₄), filtered and concentrated followed by flash column purification and recrystallization provided corresponding products.

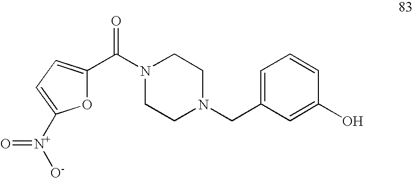

[4-(3-Hydroxy-benzyl)-piperazin-1-yl]-(5-nitro-furan-2-yl)-methanone (83). (5-Nitro-furan-2-yl)-piperazin-1-yl-methanone (400 mg, 1.77 mmol) in THF (20 mL) was added to 3-hydroxy benzaldehyde (276 mg, 1.77 mmol) followed by AcOH (2 mL) and stirred for 10 minutes at room temperature. Then Na(OAC)₃BH (525 mg, 2.48mmol) was added and the reaction mixture was stirred for 14 hrs at room temperature. The reaction was followed as explained above and column purified to yield 270 mg (45%) of compound 83;TLC: R_{f} 0.27 (ethyl acetate); ¹HNMR (500 MHz, DMSO) δ 2.42 (3H, t, J=4.88 Hz), 3.43 (2H, s), 3.46-3.81 (4H, 2bs), 6.64 (1H, dd, J=2.44, 8.3 Hz), 7.11 (1H, t, J=7.56), 7.25 (1H, d, J=3.66 Hz), 7.75 (1H, d, J=3.66 Hz), 9.33 (1H, s); ¹³CNMR (300 MHz, DMSO): 52.01, 61.63, 112.76, 114.86, 113.96, 115.54, 116.79, 119.38, 129.05, 139.06, 147.6, 156.56, 157.25; ESI-Mass: 332 (M+1).

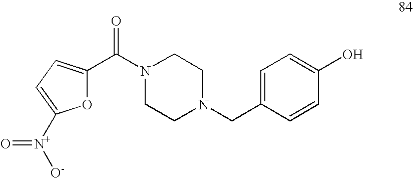

[4-(4-Hydroxy-benzyl)-piperazin-1-yl]-(5-nitro-furan-2-yl)-methanone (84). (5-Nitro-furan-2-yl)-piperazin-1-yl-methanone (400 mg, 1.77 mmol) in THF (20 mL) was added to 4-hydroxy benzaldehyde (276 mg, 1.77 mmol) followed by AcOH (2 mL) and stirred for 10 minutes at room temperature. Then Na(OAC)₃BH (525 mg, 2.48mmol) was added and the reaction mixture was stirred for 14 hrs at room temperature. The reaction was followed as explained above and column purified to yield 220 mg (37%) of compound 84;TLC: R_{f} 0.22 (ethyl acetate); ¹HNMR (500 MHz, DMSO) δ 2.39 (4H, t, J=4.88 Hz), 3.39 (2H, s), 3.51-3.7 (4H, 2bs), 6.71 (2H, d, J=8.3 Hz), 7.09, (2H, d, J=8.54 Hz), 7.25 (1H, d, J=3.9 Hz), 7.75 (1H, d, J=3.9 Hz), 9.3 (1H, s); ¹³CNMR (300 MHz, DMSO): 42.28, 46.25, 52.55, 61.17, 112.74, 114.86, 116.78, 127.5, 130.07, 147.6, 156.35, 156.53; ESI-Mass: 330 (M−1); Anal. Calcd for C₁₆H₁₇N₃O₅: C, 58.0; H, 5.17; N, 12.68. Found: C, 57.84; H, 5.26; N, 12.52.

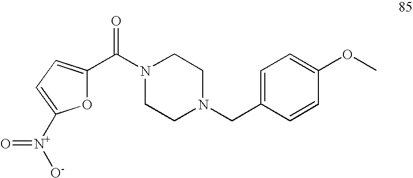

[4-(4-Methoxy-benzyl)-piperazin-1-yl]-(5-nitro-furan-2-yl)-methanone (85). (5-Nitro-furan-2-yl)-piperazin-1-yl-methanone (400 mg, 1.77 mmol) in THF (20 mL) was added to p-anisaldehyde (215 μL, 1.77 mmol) followed by AcOH (2 mL) and stirred for 10 minutes at room temperature. Then Na(OAC)₃BH (525 mg, 2.48 mmol) was added and the reaction mixture was stirred for 14 hrs at room temperature. The reaction was followed as explained above and column purified to yield 390 mg (63%) of compound 85;TLC: R_{f} 0.25 (ethyl acetate); ¹HNMR (500 MHz, DMSO) δ 2.57 (4H, t, J=4.88 Hz), 3.56 (2H, s), 3.7-3.92 (7H, m), 6.93 (2H, d, J=8.54 Hz), 7.22 (1H, d, J=3.9 Hz), 7.3 (2H, d, J=8.3 Hz), 7.55 (1H, d, J=3.9 Hz) ¹³CNMR (300 MHz, DMSO): 42.25, 46.27, 51.81, 52.54, 54.91, 60.98, 112.72, 113.51, 116.8, 129.34, 130.05, 147.6, 151.07, 156.51, 158.31; ESI-Mass: 346.1 (M+1); Anal. Calcd for C₁₇H₁₉N₃O₅: C, 59.12; H, 5.55;

N, 12.17. Found: C, 58.91; H, 5.49, N, 12.02.

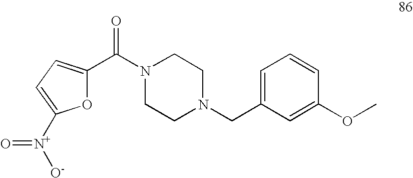

[4-(3-Methoxy-benzyl)-piperazin-1-yl]-(5-nitro-furan-2-yl)-methanone (86). (5-Nitro-furan-2-yl)-piperazin-1-yl-methanone (400 mg, 1.77 mmol) in THF (20 mL) was added to m-anisaldehyde (215 μL, 1.77 mmol) followed by AcOH (2 mL) and stirred for 10 minutes at room temperature. Then Na(OAC)₃BH (525 mg, 2.48 mmol) was added and the reaction mixture was stirred for 14 hrs at room temperature. The reaction was followed as explained above and column purified to yield 310 mg (50%) of compound 86;TLC: R_{f} 0.33 (ethyl acetate); ¹HNMR (500 MHz, DMSO) δ 2.43 (4H, t, J=4.88 Hz), 3.34 (3H, s), 3.58-3.72 (4H, m), 6.81-6.85 (1H, m), 6.88-6.85 (1H, m), 7.24 (1H, d, J=7.81 Hz), 7.26 (1H, d, J=3.9 Hz), 7.75 (1H, d, J=3.9 Hz); ¹³CNMR (300 MHz, DMSO): 42.23, 46.37, 52.71, 54.88, 61.48, 112.37, 112.75, 116.8, 120.97, 129.34, 139.24, 147.59, 156.54, 159.22; ESI-Mass: 346.1 (M+1); Anal. Calcd for C₁₇H₁₉N₃O₅: C, 59.12, H, 5.55, N, 12.17. Found: C 58.89, H, 5.49, N, 12.07.

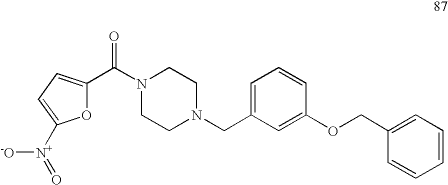

[4-(3-Benzyloxy-benzyl)-piperazin-1-yl]-(5-nitro-furan-2-yl)-methanone (87). (5-Nitro-furan-2-yl)-piperazin-1-yl-methanone (400 mg, 1.77 mmol) in THF (20 mL) was added to 3-benzyloxy benzaldehyde (375 mg, 1.77 mmol) followed by AcOH (2 mL) and stirred for 10 minutes at room temperature. Then Na(OAC)₃BH (525 mg, 2.48mmol) was added and the reaction mixture was stirred for 14 hrs at room temperature. The reaction was followed as explained above and column purified to yield 430 mg (57%) of compound 87;TLC: R_{f} 0.48 (ethyl acetate); ¹HNMR (500 MHz, DMSO) δ 2.41 (4H, t, J=5.12 Hz), 3.5 (2H, s), 3.56-3.74 (4H, 2bs), 6.88-6.92 (2H, m), 6.98-6.9(1H, bs), 7.24 (1H, d, J=8.05 Hz), 7.26 (1H, d, J=4.15 Hz), 7.32 (1H, t, J=7.32 Hz), 7.39 (2H, t, J=7.81 Hz). 7.45 (2H, d, J=7.07 Hz), 7.76 (1H, d, J=3.9 Hz) ; ¹³CNMR (300 MHz, DMSO); ESI-Mass: 444.3 (M+23); Anal. Calcd for C₁₆H₁₇N₃O₅: C, 65.55, H, 5.5; N, 9.97. Found: C, 65.25; H, 5.55; N, 9.93.

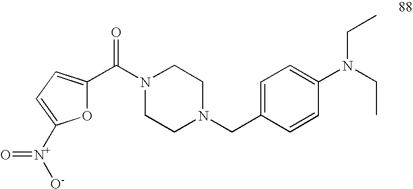

[4-(4-Diethylamino-benzyl)-piperazin-1-yl]-(5-nitro-furan-2-yl)-methanone (88). (5-Nitro-furan-2-yl)-piperazin-1-yl-methanone (400 mg, 1.77 mmol) in THF (20 mL) was added to 4-diethylamino benzaldehyde (313 mg, 1.77 mmol) followed by AcOH (2 mL) and stirred for 10 minutes at room temperature. Then Na(OAC)₃BH (525 mg, 2.48 mmol) was added and the reaction mixture was stirred for 14 hrs at room temperature. The reaction was followed as explained above and column purified to yield 290 mg (42%) of compound 88; TLC: R_{f}0.27 (ethyl acetate); ¹HNMR (500 MHz, DMSO)δ 1.18 (6H, t, J=7.08 HZ), 2.55 (4H, t, J=4.39 Hz), 3.36 (4H, q, J=7.08 Hz0, 3.61-3.91 (4H, 2bs), 6.65 (2H, d, J=8.54 Hz), 7.14 (2H, d, J=8.54 Hz), 7.16 (1H, d, J=3.66 Hz), 7.35 (1H, d, J=3.9 Hz); 13CNMR (300 MHz, DMSO) 12.36, 43.55, 61.29, 111.22, 116.77, 123.43, 130.07, 146.59, 147.63, 156.53; ESI-Mass: 409.3 (M+23); Anal. Calcd for C₂₀H₂₆N₄O₄: C, 62.16, H, 6.78, N, 14.5. Found; C, 62.07, H, 6.76, N, 14.27.

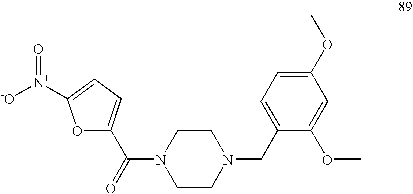

[4-(2,4-Dimethoxy-benzyl)-piperazin-1-yl]-(5-nitro-furan-2-yl)-methanone (89). (5-Nitro-furan-2-yl)-piperazin-1-yl-methanone (400 mg, 1.77 mmol) in THF (20 mL) was added to 2,4-dimethoxy benzaldehyde (294 mg, 1.77 mmol) followed by AcOH (2 mL) and stirred for 10 minutes at room temperature. Then Na(OAC)₃BH (525 mg, 2.48mmol) was added and the reaction mixture was stirred for 14 hrs at room temperature. The reaction was followed as explained above and column purified to yield 310 mg (46%) of compound 89; TLC: R_{f} 0.22 (ethyl acetate); ¹HNMR (500 MHz, DMSO) δ 2.42 (4H, t, J=4.88 Hz), 3.45 (2H, s), 3.56-3.7 (4H, 2bs), 3.75 (6H, d, J=4.15 Hz, 6.5 (1H, dd, J=2.44, 8.3 Hz), 6.54 (1H, d, J=2.44 Hz), 7.18 (1H, d, J=8.3 Hz), 7.25 (1H, d, J=3.9 Hz), 7.75 (1H, d, J=3.9 Hz); ¹³CNMR (300 MHz, DMSO) 42.32, 46.3, 51.93, 52.53, 98.22, 98.66, 104.45, 112.73, 116.78, 117.13, 130.97, 147.63, 151.09, 156.5, 158.42, 159.66; ESI-Mass: 398.1 (M+23); Anal. Calcd for C₁₈H₂₁N₃O₃: C, 57.59, H, 5.64, N, 11.19. Found; C, 57.42, H, 5.58, N, 11.07.

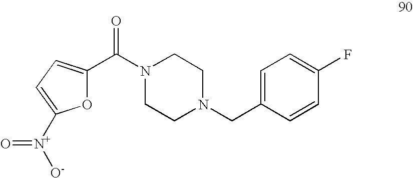

[4-(4-Fluoro-benzyl)-piperazin-1-yl]-(5-nitro-furan-2-yl)-methanone (90). (5-Nitro-furan-2-yl)-piperazin-1-yl-methanone (400 mg, 1.77 mmol) in THF (20 mL) was added to 4-floro benzaldehyde (294 mg, 1.77 mmol) followed by AcOH (2 mL) and stirred for 10 minutes at room temperature. Then Na(OAC)₃BH (525 mg, 2.48mmol) was added and the reaction mixture was stirred for 14 hrs at room temperature. The reaction was followed as explained above and column purified to yield 310 mg (65%) of compound 90; TLC: R_{f} 0.35 (ethyl acetate); ¹HNMR (500 MHz, DMSO) δ 2.42 (4H, t, J=4.88 Hz), 3.5 (2H, s), 3.58-3.76 (4H, 2bs), 7.16 (2H, t, J=8.78 Hz), 7.26 (1H, d, J=3.9 Hz) 7.36 (2H, dd, J=5.61, 8.54 Hz), 7.76 (1H, d, J=3.9 Hz); ¹³CNMR (300 MHz, DMSO) 42.25, 46.23, 51.86, 52.47, 60.61, 112.74, 114.71, 116.82, 130.6, 130.6, 133.76, 133.8, 147.58, 151.08, 156.54, 159.66, 162.87;

General Procedure for Diamides 109 and 110.

Preparation of Substituted Benzoyl Chloride.

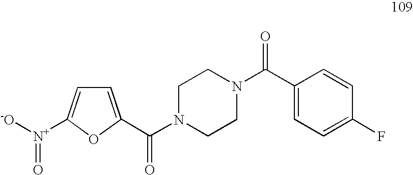

[4-(4-Fluoro-benzoyl)-piperazin-1-yl]-(5-nitro-furan-2-yl)-methanone (109). 4-Fluoro benzoic acid(249 mg, 1.77 mmol) in DCM (10 mL) was treated with oxalylchloride (308 μl, 3.54 mmol) followed by 2 drops of DMF and stirred at room temperature for 4hrs. The reaction mixture was concentrated in vacuum to obtain acid chloride and the crude 4-Fluoro-benzoyl chloride in CH₂Cl₂ (10 mL) was added to (5-Nitro-furan-2-yl)-piperazin-1-yl-methanone (400 mg, 1.77 mmol) in Et₃N (3 mL). The reaction mixture was stirred for 14 hrs at room temperature. The reaction was followed as explained above to yield 480 mg (77%) of compound 109 ; TLC: R_{f} 0.5 (ethyl acetate); ¹HNMR (500 MHz, CDCl₃): δ 3.52-4.06 (8H, 2bs), 7.16(2H, t, J=8.54 Hz), 7.26(1H, d, J=3.66 Hz), 7.38(1H, d, J=3.66 Hz), 7.48 (2H, dd, J=5.12, 8.78 Hz); ¹³CNMR (300 MHz, DMSO: CDCl₃ 6:1): 42.07, 46.02, 112.5, 115.39, 117.15, 129.58, 131.61, 147.33, 151.08, 156.74, 161.01, 164.29, 168.39; ESI-Mass: 370.8 (M+23); Anal. Calcd for C₁₆H₁₄FN₃O₅: C, 55.33; H, 4.02; N, 12.1. Found: C, 55.12; H, 4.03; N, 11.92.

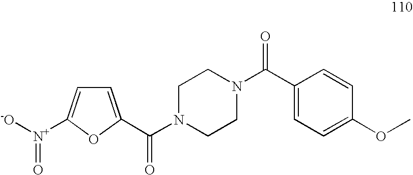

[4-(4-Methoxy-benzoyl)-piperazin-1-yl]-(5-nitro-furan-2-yl)-methanone (110). 4-Methoxy benzoic acid(269 mg, 1.77 mmol) in DCM (10 mL) was treated with oxalylchloride (308 μl, 3.54 mmol) followed by 2 drops of DMF and stirred at room temperature for 4hrs. The reaction mixture was concentrated in vacuum to obtain acid chloride and the crude 4-Methoxy-benzoyl chloride (1.77 mmol) in CH₂Cl₂ (10 mL) was added to (5-Nitro-furan-2-yl)-piperazin-1-yl-methanone (400 mg, 1.77 mmol) in Et₃N (3 mL). The reaction mixture was stirred for 14 hrs at room temperature. The reaction was followed as explained above to yield 540 mg (85%) of compound 110; TLC: R_{f} 0.5 (ethyl acetate); ¹HNMR (500 MHz, CDCl₃): δ 3.68-4.2 (11H, m), 6.96(2H, t, J=8.78 Hz), 7.25(1H, d, J=3.66 Hz), 7.38(1H, d, J=3.9 Hz), 7.44 (2H, d, J=8.78 Hz); ¹³CNMR (300 MHz, CDCl₃): 42.69, 45.99, 54.85, 111.14, 113.39, 117.97, 126.32, 128.74, 147.59, 156.33, 160.67, 170.1; ESI-Mass: 382.1 (M+23); Anal. Calcd for C₁₇H₁₇N₃O₆: C, 56.82; H, 4.77; N, 11.69. Found: C, 56.54; H, 4.74; N, 11.61.

5-Nitro-furan-2-carboxylic acid 4-[(4-methyl-piperazin-1-ylimino)-methyl]-benzylamide (111).

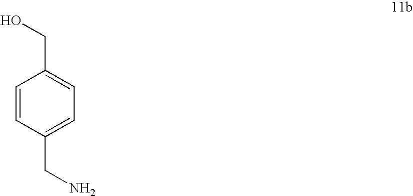

(4-Aminomethyl-phenyl)-methanol (11b). To a suspension of lithium aluminium hydride (712 mg, 18.75 mmol) in THF (40 mL) was added dropwise solution of 4-Hydroxymethyl-benzonitrile (500 mg, 3.75 mmol) in THF (10 mL), and the mixture was refluxed for 5 hrs. The reaction was mixture was cooled in as ice bath and was quenched by successive addition of water (10 mL) and 15% aqueous NaOH (6 mL). To the resulting suspension CH₂Cl₂ was added and separated from aqueous layer and filtered through celite and concentrated invacuo to obtain 11b as a solid and used for the next step without any further purification; ¹HNMR (500 MHz, CDCl₃) δ 2.2-2.8 (2H, bs), 3.75 (2H, s), 4.6 (2H,s), 7.21 (2H, d, J=7.81 Hz), 7.28 (1H, d, J=7.5 Hz).

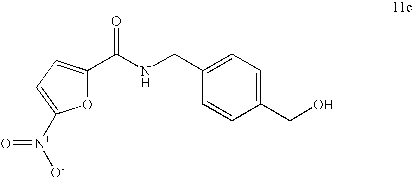

5-Nitro-furan-2-carboxylic acid 4-hydroxymethyl-benzylamide (11c). 5-Nitro-furan-2-carboxylic acid (228 mg, 1.45 mmol) in CH₂Cl₂ (25 mL) was treated with oxalylchloride (250 μL, 38.2 mmol) followed by 2 drops of DMF and stirred at room temperature for 4hrs. The reaction mixture was concentrated in vacuum to obtain acid chloride and the crude in CH₂Cl₂ (10 mL) was added to (4-Aminomethyl-phenyl)-methanol (200 mg, 1.45 mmol) in Et₃N (2.9 mmol, 205 μL). The reaction mixture was stirred for 14 hrs at room temperature. Reaction was followed by TLC. After completion of reaction 100 mL of ethyl acetate was added and washed with 10% aqueous NaHCO₃ (2×50 mL), water (2×50 mL) and brine (2×50 mL). The organic phase was dried (Na₂SO₄), filtered and concentrated followed by flash column purification to yield 260 mg (65%) of compound 11c; TLC: R_{f} 0.5(ethyl acetate); ¹HNMR (500 MHz, CDCl₃): δ 4.6 (2H, d, J=5.85 Hz), 4.68(2H, s), 7.15-7.25 (1H, bs), 7.27-7.29 (1H, m), 7.3-7.36 (4H, m), 7.37 (1H, d, J=2.68 Hz); ESI-Mass: 275 (M−1)

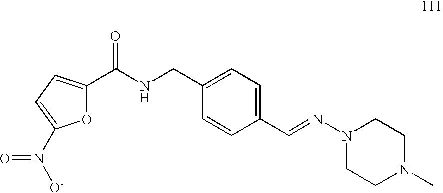

5-Nitro-furan-2-carboxylic acid 4-[(4-methyl-piperazin-1-ylimino)-methyl]-benzylamide (111). Pyridine (175 μL, 2.17 mmol) was added to 5-Nitro-furan-2-carboxylic acid 4-hydroxymethyl-benzylamide (120 mg, 0.434 mmol) in CH₂Cl₂ (10 mL) followed by Dess-Martin periodinate reagent (184 mg, 0.434 mmol). The resulting reaction mixture wa stirred at room temp. for 2.0 hrs and quenched with aqueous saturated NaHCO₃. The reaction mixture was extracted with CH₂Cl₂ and dried over Na₂SO₄ and concentrated in vacuo. To the crude (80mg, 0.291 mmol) in 10 mL of EtOH was added 4-Methyl-piperazin-1-ylamine (36 μL, 0.291 mmol). The reaction mixture was refluxed for an hour and stirred at room temperature for 14 hrs. Column purified and recrystallized to yield compound 111; TLC: R_{f} 0.4 (9:1 chloroform: methanol); ¹HNMR (500 MHz, CDCl₃) δ 2.38 (3H, s), 2.58-2.72 (4H, bs), 3.19 (4H, 2bs), 4.64 (2H, d. J=5.85 Hz), 7.31 (1H, d, J=3.66 Hz), 7.34 (1H, d, J=8.3 Hz), 7.38 (1H, d, J=3.66 Hz), 7.54 (1H, s), 7.61 (2H, d, J=8.05 Hz); ¹³CNMR (300 MHz, CDCl₃): 42.84, 45.39, 50.39, 53.92, 53.92, 111.82, 115.65, 125.98, 127.66, 134.31, 135.65, 136.11, 147.4, 155.59.

### Biological Results

To develop the structure activity relationship of compounds 72-91 and 107-111, the compounds were synthesized and tested for MIC activity against M tuberculosis (see Table 6).

The MIC of the novel compounds against M. tuberculosis H37Ra was determined by a micro broth dilution method using microtiter plates. M. tuberculosis was grown in Middlebrook 7H9 medium to an OD⁶⁵⁰ of 0.4-0.6 and a dilution made to an OD₆₅₀ of 0.01. 100 μl of these cells are then added to microtiter well containing serial dilutions of the nitrofuranyl amides. The cells, are then incubated at 37° C. for 7 days and visually examined for growth. MIC₉₀was determined for wells with greater than 90% inhibition of growth.

The MIC activity of the compounds in this example showed a strong structure-activity relationship (see Table 6). In the mono amide series the activity of the different substituents on the phenyl piperazine amides was in the order of 4-acetoxy 91>4-fluoro 108=unsubstituted 77>4-methoxy 107>2-methoxy 82. Without being limited by theory, the activity of the series appeared to show a preference for electron withdrawing substituents on the phenyl ring.

Substitution of a phenyl ring with pyrimidine 81 lead to decreased activity. Replacement of a phenylic ring with methyl 76, cyclohexyl 80, N-isopropylacetamido 78 demonstrated decreased activity, and appeared to indicate the importance of the phenyl ring.

The benzylic series were also found to exhibit good antitubercular activity. However most were less potent than the phenylic series. In the benzylic series the most active compound was 4-methoxy 85. 2,4-dimethoxy 89, 3-methoxy 86, 3-benzyloxy 87, 4-N, N diethyl amino 88, 4-fluoro 90 were all more active than unsubstituted benzyl 73. In general the diamides 109 and 110 were more active than the corresponding benzylic amides 90 and 85, which without being bound by theory appears to reflect the importance of the rigid structure in the diamides.

TABLE 6
Nitrofuranyl amides and their anti-tuberculosis activity

| No. | Compound | MIC (μg/mL) |
|---|---|---|
| 72 | 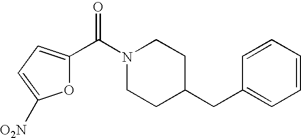 | 0.29 |
| 73 | 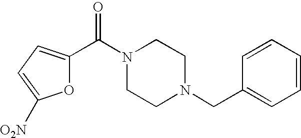 | 0.59 |
| 74 | 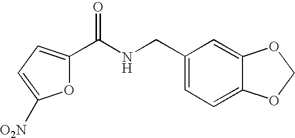 | 0.15 |
| 75 | 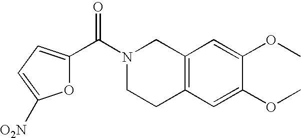 | 0.15 |
| 76 | 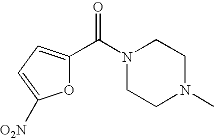 | 37.5 |
| 77 | 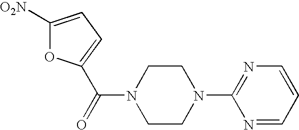 | 0.025 |
| 78 | 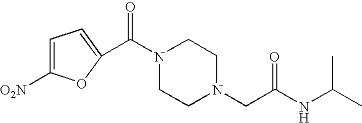 | 6.25 |
| 79 | 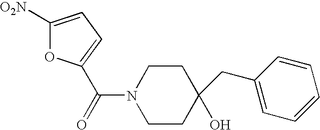 | 1.56 |
| 80 | 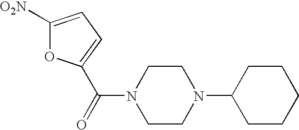 | 6.25 |
| 81 | 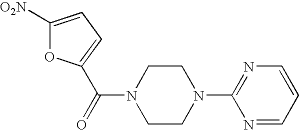 | 0.1 |
| 82 | 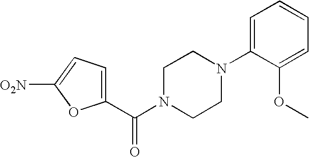 | 1.56 |
| 83 | 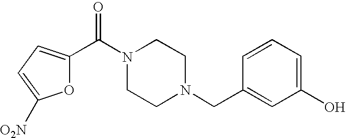 | 0.8 |
| 84 | 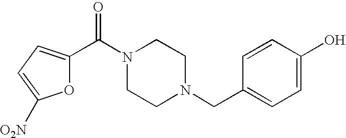 | 1.56 |
| 85 | 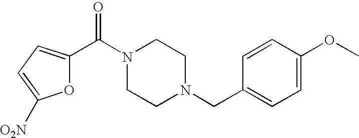 | 0.1 |
| 86 | 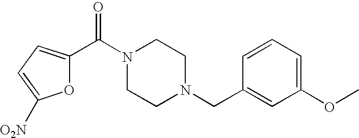 | 0.4 |
| 87 | 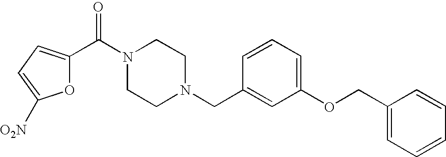 | 0.4 |
| 88 | 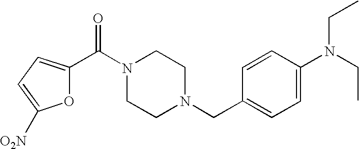 | 0.4 |
| 89 | 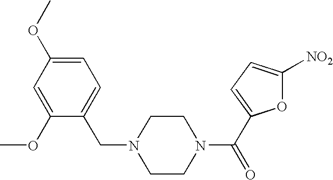 | 0.2 |
| 90 | 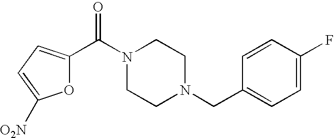 | 0.4 |
| 91 | 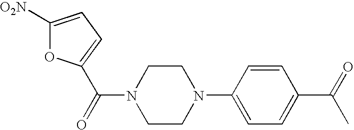 | 0.0031 |
| 107 | 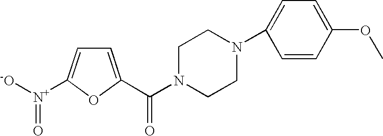 | 0.05 |
| 108 | 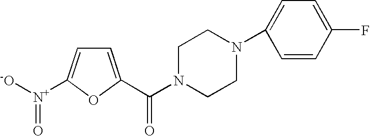 | 0.025 |
| 109 | 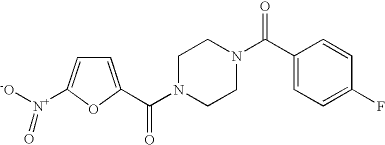 | 0.05 |
| 110 | 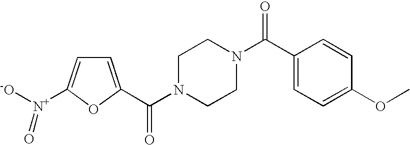 | 0.05 |
| 111 | 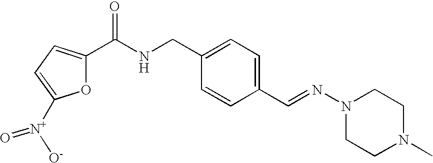 | 0.1 |

It will be understood that various details of the presently disclosed subject matter can be changed without departing from the scope of the presently disclosed subject matter. Furthermore, the foregoing description is for the purpose of illustration only, and not for the purpose of limitation.

1. A compound having the general formula: 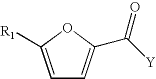
wherein R₁ is selected from the group consisting of halo, nitro, alkyl ester, arylsulfanyl, arylsulfinyl, arylsulfonyl and sulfonic acid; and Y has the formula selected from the group consisting of:
(a) —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached; 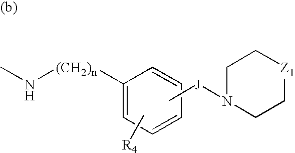
wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 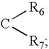
wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 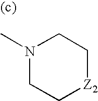
wherein Z₂ is selected from the group consisting of oxygen, NR₁₀, and 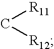
R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; and 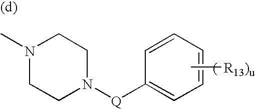
wherein Q is selected from the group consisting of (CH₂)ₛ and —C(═O)— and s is an integer from 0 to 8; u is an integer from 1 to 5; and R₁₃ is selected from the group consisting of hydrogen, halo, alkyl, substituted alkyl, aryl, and substituted aryl; or
a pharmaceutically acceptable salt thereof.

2. The compound of claim 1, wherein Y is

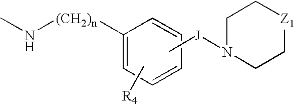

3. The compound of claim 2, wherein n is 1 and R₄ is hydrogen.

4. The compound of claim 2 wherein J is —(CH₂)_{q}—CH═N— and q is 0.

5. The compound of claim 2, wherein Z₁ is NR₅ and R₅ is an alkyl.

6. The compound of claim 2, wherein the compound is

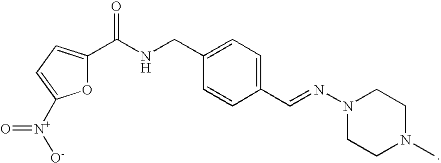

7. The compound of claim 1, wherein Y is

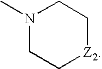

8. The compound of claim 7, wherein Z₂ is NR₁₀ and R₁₀ is a substituted aryl.

9. The compound of claim 8, wherein R₁₀ is 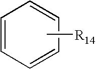
and R₁₄ is selected from the group consisting of halo, alkyl, aryl, substituted alkyl, and substituted aryl.

10. The compound of claim 7, wherein the compound is selected from the group consisting of:

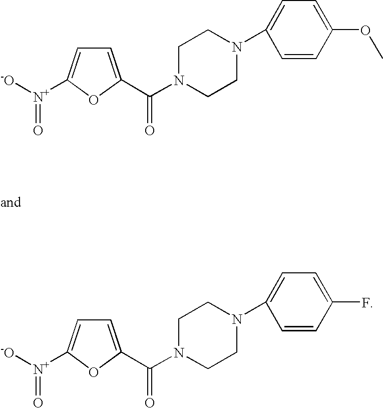

11. The compound of claim 1, wherein Y is

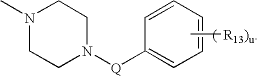

12. The compound of claim 11, wherein Q is —C(═O)—.

13. The compound of claim 11, wherein R₁₃ is selected from the group consisting of a halo an alkoxyl.

14. The compound of claim 11, wherein the compound is selected from the group consisting of:

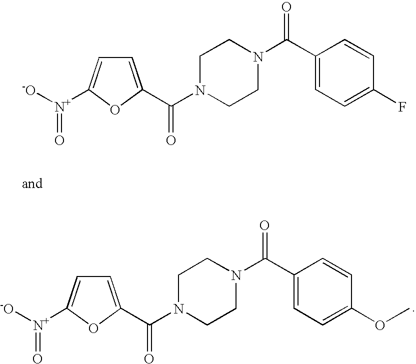

15. A method of inhibiting the growth of a microorganism comprising contacting the microorganism with an effective amount of a compound having the general formula: 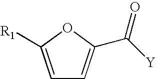
wherein R₁ is selected from the group consisting of halo, nitro, alkyl ester, arylsulfanyl, arylsulfinyl, arylsulfonyl and sulfonic acid; and Y has the formula selected from the group consisting of:
(a) —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached; 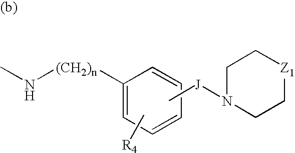
wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 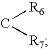
wherein R₅ is selected from the group consisting of hydrogen, alkyl, substituted alkyl, alkoxycarbonyl, aryl, substituted aryl, and —(C═O)—NR8R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 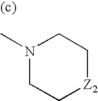
wherein Z₂ is selected from the group consisting of oxygen, NR₁₀, and 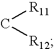
R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; and 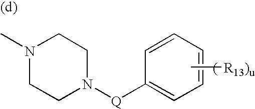
wherein Q is selected from the group consisting of (CH₂)s and —C(═O)— and s is an integer from 0 to 8; u is an integer from 1 to 5; and R₁₃ is selected from the group consisting of hydrogen, halo, alkyl, substituted alkyl, aryl, and substituted aryl; or
a pharmaceutically acceptable salt thereof.

16. The method of claim 15, wherein the microorganism is Mycobacterium tuberculosis.

17. The method of claim 15, wherein the effective amount of the compound is about 1 to about 1000 micrograms per milliliter.

18. A method of treating a microbial infection in a subject comprising administering to the subject a therapeutically effective amount of the compound having the following general formula: 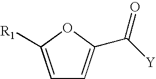
wherein R₁ is selected from the group consisting of halo, nitro, alkyl ester, arylsulfanyl, arylsulfinyl, arylsulfonyl and sulfonic acid; and Y has the formula selected from the group consisting of:
(a) —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached; 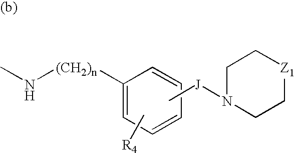
wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 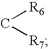
wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 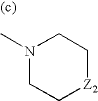
wherein Z₂ is selected from the group consisting of oxygen, NR₁₀, and 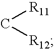
R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; and 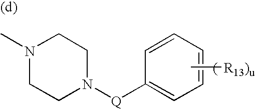
wherein Q is selected from the group consisting of (CH₂)ₛ and —C(═O)— and s is an integer from 0 to 8; u is an integer from 1 to 5; and R₁₃ is selected from the group consisting of hydrogen, halo, alkyl, substituted alkyl, aryl, and substituted aryl; or
a pharmaceutically acceptable salt thereof.

19. The method of claim 18, wherein the microbial infection is a Mycobacterium tuberculosis infection.

20. The method of claim 18, wherein the therapeutically effective amount is about 1 to about 1000 micrograms per milliliter.

21. A pharmaceutical formulation, comprising a compound having the following general structure in a pharmaceutically acceptable carrier: 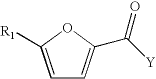
wherein R₁ is selected from the group consisting of halo, nitro, alkyl ester, arylsulfanyl, arylsulfinyl, arylsulfonyl and sulfonic acid; and Y has the formula selected from the group consisting of:
(a) —NR₂R₃, and R₂ and R₃ are each independently selected from the group consisting of H, alkyl, aryl, substituted alkyl, and substituted aryl, or R₂ and R₃ together form a ring structure with the nitrogen atom to which they are attached; 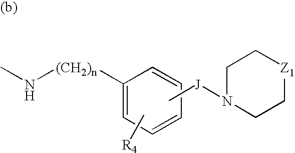
wherein n is an integer from 0 to 8; R₄ is selected from the group consisting of hydrogen, halo, alkyl, substituted alkyl, and alkoxyl; J is selected from the group consisting of —(CH₂)ₚ— and —(CH₂)_{q}—CH═N—, wherein p and q are each integers independently selected from 0 to 8; and Z₁ is selected from the group consisting of oxygen, sulfur, sulfoxide, sulfone, NR₅, and 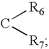
wherein R₅ is selected from the group consisting of hydrogen, alkyl, substituted alkyl, alkoxycarbonyl, aryl, substituted aryl, and —(C═O)—NR₈R₉, wherein R₆ and R₇ are each independently selected from the group consisting of hydrogen, alkyl, substituted alkyl, aryl, substituted aryl, and hydroxyl and R₈ and R₉ are each independently selected from the group consisting of hydrogen, alkyl, substituted alkyl, aryl, substituted aryl, and hydroxyl; 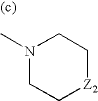
wherein Z₂ is selected from the group consisting of oxygen, NR₁₀, and 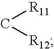
R₁₀ is selected from the group consisting of hydrogen, alkyl, substituted alkyl, aryl, and substituted aryl; and R₁₁ and R₁₂ are each independently selected from the group consisting of hydrogen, alkyl, substituted alkyl, aryl, substituted aryl, and hydroxyl; and 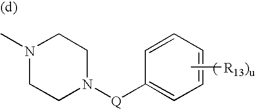
wherein Q is selected from the group consisting of (CH₂)ₛ and —C(═O)— and s is an integer from 0 to 8; u is an integer from 1 to 5; and R₁₃ is selected from the group consisting of hydrogen, halo, alkyl, substituted alkyl, aryl, and substituted aryl; or
a pharmaceutically acceptable salt thereof.
